SEQUENCE LISTING (29 sequences)

SEQ ID NO: 1 - protein, 198 aa, Influenza A virus
DTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKID

SEQ ID NO: 2 - protein, 198 aa, Influenza A virus
DTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKID

SEQ ID NO: 3 - protein, 212 aa, Influenza A virus
QKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIK

SEQ ID NO: 4 - protein, 204 aa, Influenza A virus
LDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNIN

SEQ ID NO: 5 - protein, 163 aa, Helicobacter pylori
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 6 - protein, 383 aa, Artificial Sequence
  Polypeptide sequence of H1ssF_pylori (subgenomic promoter-stabilised HA stem from A/New Caledonia/20/1999 (H1N1)-SGG-H. pylori ferritin)
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 7 - protein, 381 aa, Artificial Sequence
  Polypeptide sequence of H1ssF_pylori (subgenomic promoter-stabilised HA stem from A/Michigan/45/2015 (H1N1)-SGG-H. pylori ferritin)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 8 - protein, 394 aa, Artificial Sequence
  Polypeptide sequence of H3ssF_pylori (signal peptide-stabilised HA stem from A/Finland/486/2004 (H3N2)-SGG-H. pylori ferritin)
MKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIKSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 9 - protein, 386 aa, Artificial Sequence
  Polypeptide sequence of H10ssF_pylori (signal peptide-stabilised HA stem from A/Jiangxi/IPB13/2013 (H10N8)-SGG-H. pylori ferritin)
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 10 - protein, 203 aa, Artificial Sequence
  Polypeptide sequence of stabilised HA stem from A/Anhui/1/2013 (H7N9)
DKICLGHHAVSNGTKVNTLTERGVEVVNATELVFPGCGVLKLATGMKNVPEIPKGRGLFGAIAGFIENGWEGLIDGWYGFRHQNAQGEGTAADYKSTQSAIDQITGMVNRVIELMEQGGPDCYLAELLVAMLNQHVIDLADSEMDKLYERVKRQLRENAEEDGTGCFEIFHKCDDDCMASIRNNTYDHSKYREEAMQNRIQID

SEQ ID NO: 11 - protein, 387 aa, Artificial Sequence
  Polypeptide sequence of H7ssF_pylori (signal peptide-stabilised HA stem from A/Anhui/1/2013 (H7N9)-SGG-H. pylori ferritin)
MNTQILVFALIAIIPTNADKICLGHHAVSNGTKVNTLTERGVEVVNATELVFPGCGVLKLATGMKNVPEIPKGRGLFGAIAGFIENGWEGLIDGWYGFRHQNAQGEGTAADYKSTQSAIDQITGMVNRVIELMEQGGPDCYLAELLVAMLNQHVIDLADSEMDKLYERVKRQLRENAEEDGTGCFEIFHKCDDDCMASIRNNTYDHSKYREEAMQNRIQIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 12 - protein, 263 aa, Artificial Sequence
  Polypeptide sequence of H1ssF_TM (signal peptide-stabilised HA stem from A/Michigan/45/2015 (H1N1)-SGG-transmembrane element)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 13 - protein, 275 aa, Artificial Sequence
  Polypeptide sequence of H3ssF_TM (signal peptide-stabilised HA stem from A/Finland/486/2004 (H3N2)-SGG- transmembrane element)
MKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 14 - protein, 267 aa, Artificial Sequence
  Polypeptide sequence of H10ssF_TM (signal peptide-stabilised HA stem from A/Jiangxi/IPB13/2013 (H10N8)-SGG- transmembrane element)
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINPVTLSSGYKDIILWFSFGASCFVLLAVVMGLFFFCLKNGNMRCTICI

SEQ ID NO: 15 - protein, 268 aa, Artificial Sequence
  Polypeptide sequence of H7ssF_TM (signal peptide-stabilised HA stem from A/Anhui/1/2013 (H7N9)-SGG- transmembrane element)
MNTQILVFALIAIIPTNADKICLGHHAVSNGTKVNTLTERGVEVVNATELVFPGCGVLKLATGMKNVPEIPKGRGLFGAIAGFIENGWEGLIDGWYGFRHQNAQGEGTAADYKSTQSAIDQITGMVNRVIELMEQGGPDCYLAELLVAMLNQHVIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 16 - RNA, 1418 nt, Artificial Sequence
  Nucleic acid sequence of unmodified nativeSP_H1ss_pylori from A/Michigan/45/2015 (H1N1)
aggagagucccgcagucggcguccagcggcucugcuuguucgugugugugucguugcaggccuuauucaagcuuaccaugaaggccauccuggugguccuccuguacaccuucaccacggccaacgcggacacccugugcaucggcuaccacgccaacaacagcaccgacaccguggacaccguccucgagaagaacgugaccgugacccacuccgucaaccuggggagcggccugcgccucgccacggggcugcggaacgugcccuccauccagagccgcggccuguucggggcgaucgccggcuucaucgagggcggguggaccggcaugguggacgggugguacggcuaccaccaccagaacgagcagggguccggcuacgccgccgaccucaagagcacccagaacgccaucgacaagaucaccaacauggucaacuccgugaucgagaagaugggcagcgggggcuccgggacguacaacgcggagcugcuggugcuccugcugaacgagcggacccucgacuaccacgacagcaacgucaagaaccuguacgagaaggugcgcaaccagcugaagaacaacgccaaggagaucggcaacggcugcuucgaguucuaccacaagugcgacaacacgugcauggaguccgugaagaacgggaccuacgacuacccgaaguacagcgaggaggccaagcucaaccgggagaagaucgacagcggcggggacaucaucaagcuccugaacgagcaggucaacaaggagaugcaguccagcaaccuguacauguccaugagcuccuggugcuacacccacagccucgacggcgccgggcuguuccuguucgaccacgccgcggaggaguacgagcacgccaagaagcucaucaucuuccugaacgagaacaacgugcccgugcagcugaccuccaucagcgccccggagcacaaguucgagggccucacgcagaucuuccagaaggccuacgagcacgagcagcacaucuccgagagcaucaacaacaucguggaccacgccaucaaguccaaggaccacgcgaccuucaacuuccugcagugguacgucgccgagcagcacgaggaggaggugcuguucaaggacauccucgacaagaucgagcugaucgggaacgagaaccacggccuguaccucgccgaccaguacgugaaggggaucgccaagagccgcaaguccugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 1424 nt, Artificial Sequence
  Nucleic acid sequence of N1-methylpseudouridine modified nativeSP_H1ss_pylori from A/Michigan/45/2015 (H1N1)
aggagagucccgcagucggcguccagcggcucugcuuguucgugugugugucguugcaggccuuauucaagcuuaccaugaaggccauccuggugguccuccuguacaccuucaccacggccaacgcggacacccugugcaucggcuaccacgccaacaacagcaccgacaccguggacaccguccucgagaagaacgugaccgugacccacuccgucaaccuggggagcggccugcgccucgccacggggcugcggaacgugcccuccauccagagccgcggccuguucggggcgaucgccggcuucaucgagggcggguggaccggcaugguggacgggugguacggcuaccaccaccagaacgagcagggguccggcuacgccgccgaccucaagagcacccagaacgccaucgacaagaucaccaacauggucaacuccgugaucgagaagaugggcagcgggggcuccgggacguacaacgcggagcugcuggugcuccugcugaacgagcggacccucgacuaccacgacagcaacgucaagaaccuguacgagaaggugcgcaaccagcugaagaacaacgccaaggagaucggcaacggcugcuucgaguucuaccacaagugcgacaacacgugcauggaguccgugaagaacgggaccuacgacuacccgaaguacagcgaggaggccaagcucaaccgggagaagaucgacagcggcggggacaucaucaagcuccugaacgagcaggucaacaaggagaugcaguccagcaaccuguacauguccaugagcuccuggugcuacacccacagccucgacggcgccgggcuguuccuguucgaccacgccgcggaggaguacgagcacgccaagaagcucaucaucuuccugaacgagaacaacgugcccgugcagcugaccuccaucagcgccccggagcacaaguucgagggccucacgcagaucuuccagaaggccuacgagcacgagcagcacaucuccgagagcaucaacaacaucguggaccacgccaucaaguccaaggaccacgcgaccuucaacuuccugcagugguacgucgccgagcagcacgaggaggaggugcuguucaaggacauccucgacaagaucgagcugaucgggaacgagaaccacggccuguaccucgccgaccaguacgugaaggggaucgccaagagccgcaaguccugaugaugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - RNA, 1457 nt, Artificial Sequence
  Nucleic acid sequence of unmodified nativeSP_H3ss_pylori from A/Finland/486/2004 (H3N2)
aggagagucccgcagucggcguccagcggcucugcuuguucgugugugugucguugcaggccuuauucaagcuuaccaugaagaccaucaucgcccugagcuacauccucugccugguguucgcccagaagcugcccggcaacgacaacagcaccgccacccucugccuggggcaccacgccgugccaaacggcacaaucgucaagaccaucaccaacgaccagaucgaggugaccaacgcaacagagcugguguuccccgggugcggcguccucaagcuggccaccgggaugaggaacgugcccgagaagcagaccaggggcaucuucggcgccaucgccggguucaucgagaacggcugggaggggaugguggacggcugguacggguucaggcaccagaacuccgagggcaucggccaggccgcagaccucaagagcacccaggccgccaucaaccagaucaacgggauggucaaccgggugaucgagcugauggagcagggcgggcccgacugcuaccuggccgagcugcucguggcacugcugaaccagcacgucaucgaccucacagacuccgagaugaggaagcuguucgagcggaccaagaagcagcugagggagaacgccgaggacaugggcaacggcugcuucaagaucuaccacaagugcgacaacgccugcaucgggagcauccggaacggcaccuacgaccacgacguguacagggacgaggcccucaacaaccgguuccagaucaagagcggcggggacaucaucaagcuccugaacgagcaggucaacaaggagaugcaguccagcaaccuguacauguccaugagcuccuggugcuacacccacagccucgacggcgccgggcuguuccuguucgaccacgccgcggaggaguacgagcacgccaagaagcucaucaucuuccugaacgagaacaacgugcccgugcagcugaccuccaucagcgccccggagcacaaguucgagggccucacgcagaucuuccagaaggccuacgagcacgagcagcacaucuccgagagcaucaacaacaucguggaccacgccaucaaguccaaggaccacgcgaccuucaacuuccugcagugguacgucgccgagcagcacgaggaggaggugcuguucaaggacauccucgacaagaucgagcugaucgggaacgagaaccacggccuguaccucgccgaccaguacgugaaggggaucgccaagagccgcaaguccugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - RNA, 1463 nt, Artificial Sequence
  Nucleic acid sequence of N1-methylpseudouridine modified nativeSP_H3ss_pylori from A/Finland/486/2004 (H3N2)
aggagagucccgcagucggcguccagcggcucugcuuguucgugugugugucguugcaggccuuauucaagcuuaccaugaagaccaucaucgcccugagcuacauccucugccugguguucgcccagaagcugcccggcaacgacaacagcaccgccacccucugccuggggcaccacgccgugccaaacggcacaaucgucaagaccaucaccaacgaccagaucgaggugaccaacgcaacagagcugguguuccccgggugcggcguccucaagcuggccaccgggaugaggaacgugcccgagaagcagaccaggggcaucuucggcgccaucgccggguucaucgagaacggcugggaggggaugguggacggcugguacggguucaggcaccagaacuccgagggcaucggccaggccgcagaccucaagagcacccaggccgccaucaaccagaucaacgggauggucaaccgggugaucgagcugauggagcagggcgggcccgacugcuaccuggccgagcugcucguggcacugcugaaccagcacgucaucgaccucacagacuccgagaugaggaagcuguucgagcggaccaagaagcagcugagggagaacgccgaggacaugggcaacggcugcuucaagaucuaccacaagugcgacaacgccugcaucgggagcauccggaacggcaccuacgaccacgacguguacagggacgaggcccucaacaaccgguuccagaucaagagcggcggggacaucaucaagcuccugaacgagcaggucaacaaggagaugcaguccagcaaccuguacauguccaugagcuccuggugcuacacccacagccucgacggcgccgggcuguuccuguucgaccacgccgcggaggaguacgagcacgccaagaagcucaucaucuuccugaacgagaacaacgugcccgugcagcugaccuccaucagcgccccggagcacaaguucgagggccucacgcagaucuuccagaaggccuacgagcacgagcagcacaucuccgagagcaucaacaacaucguggaccacgccaucaaguccaaggaccacgcgaccuucaacuuccugcagugguacgucgccgagcagcacgaggaggaggugcuguucaaggacauccucgacaagaucgagcugaucgggaacgagaaccacggccuguaccucgccgaccaguacgugaaggggaucgccaagagccgcaaguccugaugaugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - RNA, 1439 nt, Artificial Sequence
  Nucleic acid sequence of N1-methylpseudouridine modified nativeSP_H10ss_pylori from A/Jiangxi/IPB13/2013 (H10N8)
aggagagucccgcagucggcguccagcggcucugcuuguucgugugugugucguugcaggccuuauucaagcuuaccauguacaagaucguggucaucaucgcccugcucggcgccgugaaggggcuggacaagaucugccucggccaccacgccguggccaacggaaccaucgucaagacccugacaaacgagcaggaggaggugaccaacgcaaccgagcugguguuccccggcugcggaguccucaugcuggccaccggcaugaggaacgugccagagcugauccagggaaggggccucuucggcgccaucgccggauuccuggagaacggcugggagggaaugguggacggcugguacggauucaggcaccagaacgcccagggcacaggccaggcagccgacuacaagagcacccaggccgccaucgaccagaucaccggaauggucaacagggugguggagcugauggagcagggcgggcccgacugcuaccucgcagagcugcuggucgccaugcucaaccagcacgugaucgacauggccgacuccgagaugaggaaccuguacgagagggugaggaagcagcugaggcagaacgccgaggaggacggcaagggcugcuucgagaucuaccacgccugcgacgacagcugcauggaguccaucaggaacaacaccuacgaccacagccaguacagggaggaggcacuccugaacaggcugaacaucaacagcggcggggacaucaucaagcuccugaacgagcaggucaacaaggagaugcaguccagcaaccuguacauguccaugagcuccuggugcuacacccacagccucgacggcgccgggcuguuccuguucgaccacgccgcggaggaguacgagcacgccaagaagcucaucaucuuccugaacgagaacaacgugcccgugcagcugaccuccaucagcgccccggagcacaaguucgagggccucacgcagaucuuccagaaggccuacgagcacgagcagcacaucuccgagagcaucaacaacaucguggaccacgccaucaaguccaaggaccacgcgaccuucaacuuccugcagugguacgucgccgagcagcacgaggaggaggugcuguucaaggacauccucgacaagaucgagcugaucgggaacgagaaccacggccuguaccucgccgaccaguacgugaaggggaucgccaagagccgcaaguccugaugaugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 21 - RNA, 1442 nt, Artificial Sequence
  Nucleic acid sequence of N1-methylpseudouridine modified nativeSP_H7ss_pylori from A/Anhui/1/2013 (H7N9)
aggagagucccgcagucggcguccagcggcucugcuuguucgugugugugucguugcaggccuuauucaagcuuaccaugaacacccagauccugguguucgcccucaucgccaucauccccaccaacgcggacaagaucugccugggccaccacgccgugagcaacggaaccaaggucaacacccucacagagaggggcguggagguggucaacgccaccgagcugguguuccccggaugcggcgugcugaagcucgcaaccggaaugaagaacgucccagagauccccaagggcaggggccuguucggagccaucgccggcuucaucgagaacgggugggagggccugaucgacgggugguacggcuucaggcaccagaacgcccagggcgaggggaccgccgcagacuacaaguccacacagagcgccaucgaccagaucaccggcauggugaacagggugaucgagcucauggagcagggcgggcccgacugcuaccuggccgagcugcucgucgccaugcugaaccagcacgugaucgaccuggcagacuccgagauggacaagcucuacgagagggugaagaggcagcugagggagaacgccgaggaggacggcaccgggugcuucgagaucuuccacaagugcgacgacgacugcauggccagcaucaggaacaacaccuacgaccacuccaaguacagggaggaggccaugcagaacaggauccagaucgacagcggcggggacaucaucaagcuccugaacgagcaggucaacaaggagaugcaguccagcaaccuguacauguccaugagcuccuggugcuacacccacagccucgacggcgccgggcuguuccuguucgaccacgccgcggaggaguacgagcacgccaagaagcucaucaucuuccugaacgagaacaacgugcccgugcagcugaccuccaucagcgccccggagcacaaguucgagggccucacgcagaucuuccagaaggccuacgagcacgagcagcacaucuccgagagcaucaacaacaucguggaccacgccaucaaguccaaggaccacgcgaccuucaacuuccugcagugguacgucgccgagcagcacgaggaggaggugcuguucaaggacauccucgacaagaucgagcugaucgggaacgagaaccacggccuguaccucgccgaccaguacgugaaggggaucgccaagagccgcaaguccugaugaugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - RNA, 1064 nt, Artificial Sequence
  Nucleic acid sequence of unmodified nativeSP_H1ss_TM from A/Michigan/45/2015 (H1N1)
aggagagucccgcagucggcguccagcggcucugcuuguucgugugugugucguugcaggccuuauucaagcuuaccaugaaggccauccuggugguccuccuguacaccuucaccacggccaacgcggacacccugugcaucggcuaccacgccaacaacagcaccgacaccguggacaccguccucgagaagaacgugaccgugacccacuccgucaaccuggggagcggccugcgccucgccacggggcugcggaacgugcccuccauccagagccgcggccuguucggggcgaucgccggcuucaucgagggcggguggaccggcaugguggacgggugguacggcuaccaccaccagaacgagcagggguccggcuacgccgccgaccucaagagcacccagaacgccaucgacaagaucaccaacauggucaacuccgugaucgagaagaugggcagcgggggcuccgggacguacaacgcggagcugcuggugcuccugcugaacgagcggacccucgacuaccacgacagcaacgucaagaaccuguacgagaaggugcgcaaccagcugaagaacaacgccaaggagaucggcaacggcugcuucgaguucuaccacaagugcgacaacacgugcauggaguccgugaagaacgggaccuacgacuacccgaaguacagcgaggaggccaagcucaaccgggagaagaucgacggcgugaagcucgagagcacccggaucuaccagauccuggccaucuacuccaccgucgccagcucccuggugcucguggucagccugggggcgaucuccuucuggaugugcagcaacggcagccugcagugccgcaucugcaucugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - RNA, 1070 nt, Artificial Sequence
  Nucleic acid sequence of N1-methylpseudouridine modified nativeSP_H1ss_TM from A/Michigan/45/2015 (H1N1)
aggagagucccgcagucggcguccagcggcucugcuuguucgugugugugucguugcaggccuuauucaagcuuaccaugaaggccauccuggugguccuccuguacaccuucaccacggccaacgcggacacccugugcaucggcuaccacgccaacaacagcaccgacaccguggacaccguccucgagaagaacgugaccgugacccacuccgucaaccuggggagcggccugcgccucgccacggggcugcggaacgugcccuccauccagagccgcggccuguucggggcgaucgccggcuucaucgagggcggguggaccggcaugguggacgggugguacggcuaccaccaccagaacgagcagggguccggcuacgccgccgaccucaagagcacccagaacgccaucgacaagaucaccaacauggucaacuccgugaucgagaagaugggcagcgggggcuccgggacguacaacgcggagcugcuggugcuccugcugaacgagcggacccucgacuaccacgacagcaacgucaagaaccuguacgagaaggugcgcaaccagcugaagaacaacgccaaggagaucggcaacggcugcuucgaguucuaccacaagugcgacaacacgugcauggaguccgugaagaacgggaccuacgacuacccgaaguacagcgaggaggccaagcucaaccgggagaagaucgacggcgugaagcucgagagcacccggaucuaccagauccuggccaucuacuccaccgucgccagcucccuggugcucguggucagccugggggcgaucuccuucuggaugugcagcaacggcagccugcagugccgcaucugcaucugaugaugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 24 - RNA, 1100 nt, Artificial Sequence
  Nucleic acid sequence of unmodified nativeSP_H3ss_TM from A/Finland/486/2004 (H3N2)
aggagagucccgcagucggcguccagcggcucugcuuguucgugugugugucguugcaggccuuauucaagcuuaccaugaagaccaucaucgcccugagcuacauccucugccugguguucgcccagaagcugcccggcaacgacaacagcaccgccacccucugccuggggcaccacgccgugccaaacggcacaaucgucaagaccaucaccaacgaccagaucgaggugaccaacgcaacagagcugguguuccccgggugcggcguccucaagcuggccaccgggaugaggaacgugcccgagaagcagaccaggggcaucuucggcgccaucgccggguucaucgagaacggcugggaggggaugguggacggcugguacggguucaggcaccagaacuccgagggcaucggccaggccgcagaccucaagagcacccaggccgccaucaaccagaucaacgggauggucaaccgggugaucgagcugauggagcagggcgggcccgacugcuaccuggccgagcugcucguggcacugcugaaccagcacgucaucgaccucacagacuccgagaugaggaagcuguucgagcggaccaagaagcagcugagggagaacgccgaggacaugggcaacggcugcuucaagaucuaccacaagugcgacaacgccugcaucgggagcauccggaacggcaccuacgaccacgacguguacagggacgaggcccucaacaaccgguuccagaucaagggcgucgagcucaagagcggcuacaaggacuggauccuguggaucuccuucgcgaucagcugcuuccugcugugcguggcccugcucggguucaucaugugggccugccagaagggcaacauccggugcaacaucugcaucugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 25 - RNA, 1106 nt, Artificial Sequence
  Nucleic acid sequence of N1-methylpseudouridine modified nativeSP_H3ss_TM from A/Finland/486/2004 (H3N2)
aggagagucccgcagucggcguccagcggcucugcuuguucgugugugugucguugcaggccuuauucaagcuuaccaugaagaccaucaucgcccugagcuacauccucugccugguguucgcccagaagcugcccggcaacgacaacagcaccgccacccucugccuggggcaccacgccgugccaaacggcacaaucgucaagaccaucaccaacgaccagaucgaggugaccaacgcaacagagcugguguuccccgggugcggcguccucaagcuggccaccgggaugaggaacgugcccgagaagcagaccaggggcaucuucggcgccaucgccggguucaucgagaacggcugggaggggaugguggacggcugguacggguucaggcaccagaacuccgagggcaucggccaggccgcagaccucaagagcacccaggccgccaucaaccagaucaacgggauggucaaccgggugaucgagcugauggagcagggcgggcccgacugcuaccuggccgagcugcucguggcacugcugaaccagcacgucaucgaccucacagacuccgagaugaggaagcuguucgagcggaccaagaagcagcugagggagaacgccgaggacaugggcaacggcugcuucaagaucuaccacaagugcgacaacgccugcaucgggagcauccggaacggcaccuacgaccacgacguguacagggacgaggcccucaacaaccgguuccagaucaagggcgucgagcucaagagcggcuacaaggacuggauccuguggaucuccuucgcgaucagcugcuuccugcugugcguggcccugcucggguucaucaugugggccugccagaagggcaacauccggugcaacaucugcaucugaugaugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - RNA, 1076 nt, Artificial Sequence
  Nucleic acid sequence of unmodified nativeSP_H10ss_TM from A/Jiangxi/IPB13/2013 (H10N8)
aggagagucccgcagucggcguccagcggcucugcuuguucgugugugugucguugcaggccuuauucaagcuuaccauguacaagaucguggucaucaucgcccugcucggcgccgugaaggggcuggacaagaucugccucggccaccacgccguggccaacggaaccaucgucaagacccugacaaacgagcaggaggaggugaccaacgcaaccgagcugguguuccccggcugcggaguccucaugcuggccaccggcaugaggaacgugccagagcugauccagggaaggggccucuucggcgccaucgccggauuccuggagaacggcugggagggaaugguggacggcugguacggauucaggcaccagaacgcccagggcacaggccaggcagccgacuacaagagcacccaggccgccaucgaccagaucaccggaauggucaacagggugguggagcugauggagcagggcgggcccgacugcuaccucgcagagcugcuggucgccaugcucaaccagcacgugaucgacauggccgacuccgagaugaggaaccuguacgagagggugaggaagcagcugaggcagaacgccgaggaggacggcaagggcugcuucgagaucuaccacgccugcgacgacagcugcauggaguccaucaggaacaacaccuacgaccacagccaguacagggaggaggcacuccugaacaggcugaacaucaaccccgucacgcuguccagcggcuacaaggacaucauccugugguucuccuucggggcgagcugcuucgugcuccuggccgucgugaugggccuguucuucuucugccucaagaacggcaacaugcgcugcaccaucugcaucugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - RNA, 1082 nt, Artificial Sequence
  Nucleic acid sequence of N1-methylpseudouridine modified nativeSP_H10ss_TM from A/Jiangxi/IPB13/2013 (H10N8)
aggagagucccgcagucggcguccagcggcucugcuuguucgugugugugucguugcaggccuuauucaagcuuaccauguacaagaucguggucaucaucgcccugcucggcgccgugaaggggcuggacaagaucugccucggccaccacgccguggccaacggaaccaucgucaagacccugacaaacgagcaggaggaggugaccaacgcaaccgagcugguguuccccggcugcggaguccucaugcuggccaccggcaugaggaacgugccagagcugauccagggaaggggccucuucggcgccaucgccggauuccuggagaacggcugggagggaaugguggacggcugguacggauucaggcaccagaacgcccagggcacaggccaggcagccgacuacaagagcacccaggccgccaucgaccagaucaccggaauggucaacagggugguggagcugauggagcagggcgggcccgacugcuaccucgcagagcugcuggucgccaugcucaaccagcacgugaucgacauggccgacuccgagaugaggaaccuguacgagagggugaggaagcagcugaggcagaacgccgaggaggacggcaagggcugcuucgagaucuaccacgccugcgacgacagcugcauggaguccaucaggaacaacaccuacgaccacagccaguacagggaggaggcacuccugaacaggcugaacaucaaccccgucacgcuguccagcggcuacaaggacaucauccugugguucuccuucggggcgagcugcuucgugcuccuggccgucgugaugggccuguucuucuucugccucaagaacggcaacaugcgcugcaccaucugcaucugaugaugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - RNA, 1079 nt, Artificial Sequence
  Nucleic acid sequence of unmodified nativeSP_H7ss_TM from A/Anhui/1/2013 (H7N9)
aggagagucccgcagucggcguccagcggcucugcuuguucgugugugugucguugcaggccuuauucaagcuuaccaugaacacccagauccugguguucgcccucaucgccaucauccccaccaacgcggacaagaucugccugggccaccacgccgugagcaacggaaccaaggucaacacccucacagagaggggcguggagguggucaacgccaccgagcugguguuccccggaugcggcgugcugaagcucgcaaccggaaugaagaacgucccagagauccccaagggcaggggccuguucggagccaucgccggcuucaucgagaacgggugggagggccugaucgacgggugguacggcuucaggcaccagaacgcccagggcgaggggaccgccgcagacuacaaguccacacagagcgccaucgaccagaucaccggcauggugaacagggugaucgagcucauggagcagggcgggcccgacugcuaccuggccgagcugcucgucgccaugcugaaccagcacgugaucgaccuggcagacuccgagauggacaagcucuacgagagggugaagaggcagcugagggagaacgccgaggaggacggcaccgggugcuucgagaucuuccacaagugcgacgacgacugcauggccagcaucaggaacaacaccuacgaccacuccaaguacagggaggaggccaugcagaacaggauccagaucgaccccgugaagcugagcuccggcuacaaggacgucauccucugguucagcuucggggccuccugcuucauccugcuggccaucgugaugggccucguguucaucugcgucaagaacgggaacaugcgcugcaccaucugcaucugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 1085 nt, Artificial Sequence
  Nucleic acid sequence of N1-methylpseudouridine modified nativeSP_H7ss_TM from A/Anhui/1/2013 (H7N9)
aggagagucccgcagucggcguccagcggcucugcuuguucgugugugugucguugcaggccuuauucaagcuuaccaugaacacccagauccugguguucgcccucaucgccaucauccccaccaacgcggacaagaucugccugggccaccacgccgugagcaacggaaccaaggucaacacccucacagagaggggcguggagguggucaacgccaccgagcugguguuccccggaugcggcgugcugaagcucgcaaccggaaugaagaacgucccagagauccccaagggcaggggccuguucggagccaucgccggcuucaucgagaacgggugggagggccugaucgacgggugguacggcuucaggcaccagaacgcccagggcgaggggaccgccgcagacuacaaguccacacagagcgccaucgaccagaucaccggcauggugaacagggugaucgagcucauggagcagggcgggcccgacugcuaccuggccgagcugcucgucgccaugcugaaccagcacgugaucgaccuggcagacuccgagauggacaagcucuacgagagggugaagaggcagcugagggagaacgccgaggaggacggcaccgggugcuucgagaucuuccacaagugcgacgacgacugcauggccagcaucaggaacaacaccuacgaccacuccaaguacagggaggaggccaugcagaacaggauccagaucgaccccgugaagcugagcuccggcuacaaggacgucauccucugguucagcuucggggccuccugcuucauccugcuggccaucgugaugggccucguguucaucugcgucaagaacgggaacaugcgcugcaccaucugcaucugaugaugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0181037 A1
(43) Pub. Date: Jun. 6, 2024

(54) IMMUNOGENIC COMPOSITIONS

(71) Applicants: GLAXOSMITHKLINE BIOLOGICALS SA, Rixensart (BE); CUREVAC SE, Tübingen (DE)

(72) Inventors: Hans Wolfgang GROßE, Tübingen (DE); Edith JASNY, Tübingen (DE); Janine MÜHE, Tübingen (DE); Ventzislav Bojidarov VASSILEV, Rixensart (BE); Clarisse LORIN, Rixensart (BE); Nadia OUAKED, Rixensart (BE); Corey MALLETT, Rockville, MD (US); Ronan ROUXEL, Rixensart (BE); Normand BLAIS, Rixensart (BE)

(73) Assignees: GLAXOSMITHKLINE BIOLOGICALS SA, Rixensart (BE); CUREVAC SE, Tübingen (DE)

(21) Appl. No.: 18/283,601

(22) PCT Filed: Mar. 25, 2022
(86) PCT No.: PCT/EP2022/057934

§ 371 Date: Sep. 22, 2023

Related U.S. Application Data
(60) Provisional application No. 63/166,539, filed on Mar. 26, 2021.

(51) Int. Cl. A61K 39/145; A61K 39/00; A61P 31/16; A61P 37/04

(52) U.S. Cl. CPC A61K 39/145; A61P 31/16; A61P 37/04; A61K 2039/53; A61K 2039/6018; A61K 2039/6031; A61K 2039/6093

(57) ABSTRACT

The present invention relates to carrier-formulated mRNA comprising at least one coding sequence encoding an influenza HA stem polypeptide, and to related aspects.

The present application claims priority to U.S. provisional application No. 63/166,539 filed on Mar. 26, 2021, the contents of which are incorporated by reference in their entirety.

This invention was created in the performance of a Cooperative Research and Development Agreement with the National Institutes of Health, an Agency of the Department of Health and Human Services. The Government of the United States has certain rights in this invention.

### TECHNICAL FIELD

The present invention relates to influenza immunisation using a hemagglutinin (HA) stem polypeptide delivered in the form of carrier-formulated mRNA, and to related aspects.

### BACKGROUND

Influenza viruses have a significant impact on global public health, causing millions of cases of severe illness each year, thousands of deaths, and considerable economic losses. Current tri- or tetravalent influenza vaccines elicit antibody responses to the vaccine strains and closely related isolates, but rarely extend to more diverged strains within a subtype or to other subtypes. In addition, selection of the appropriate vaccine strains presents many challenges and frequently results in sub-optimal protection.

Protective immune responses induced by vaccination against influenza viruses are primarily directed to the viral HA protein, which is a glycoprotein on the surface of the virus responsible for interaction of the virus with host cell receptors. HA proteins on the virus surface are trimers of HA protein monomers that are enzymatically cleaved to yield amino-terminal HA1 and carboxy-terminal HA2 polypeptides. The globular head consists exclusively of the major portion of the HA1 polypeptide, whereas the stem that anchors the HA protein into the viral lipid envelope is comprised of HA2 and part of HA1. The globular head of a HA protein includes two domains: the receptor binding domain (RBD), a domain that includes the sialic acid-binding site, and the vestigial esterase domain, a smaller region just below the RBD. The globular head includes several antigenic sites that include immunodominant epitopes.

Therefore, antibodies against influenza often target variable antigenic sites in the globular head of HA and thus, neutralize only antigenically closely related viruses. The variability of the HA head is due to the constant antigenic drift (i.e., changes in the protein sequence) of influenza viruses and is responsible for seasonal endemics of influenza. Based on the sequence of HA and that of the other surface glycoprotein neuraminidase (NA), which is also affected by antigenic drift, influenza virus strains are classified into different subtypes. In total, 18 HAs and 11 NAs have been isolated thus far and are further divided into two groups each, e.g. HA group 1 contains e.g. H1, H2, H5, and H9 and group 2 contains e.g. H3, H7, and H10.

In contrast to the HA-head, the HA stem is highly conserved and experiences little antigenic drift.

In fact, an entirely new class of broadly neutralizing antibodies against influenza viruses has been isolated that recognize the highly conserved HA stem (Corti, 2011). Unlike strain-specific antibodies, antibodies in this new class are capable of neutralizing multiple antigenically distinct viruses. However, robustly eliciting these antibodies in subjects by vaccination with the HA stem, lacking the head domain, has been difficult (Steel, 2010). Removal of the immunodominant head region of HA (which contains competing epitopes) and stabilization of the resulting stem region through genetic manipulation is one potential way to improve the elicitation of these broadly neutralizing stem antibodies.

Advances in biotechnology in past decades have allowed engineering of biological materials to be exploited for the generation of novel vaccine platforms. Ferritin, an iron storage protein found in almost all living organisms, is an example which has been extensively studied and engineered for a number of potential biochemical/biomedical purposes. The use of ferritin self-assembling nanoparticles to present stabilised stem trimers is described in Corbett, 2019.

Messenger RNA (mRNA) is a single-stranded RNA molecule that corresponds to the genetic sequence of a gene and is read by ribosomes in the process of producing a protein. mRNA based vaccines provide an alternative vaccination approach to traditional strategies involving live attenuated/inactivated pathogens or subunit vaccines (Zhang, 2019). mRNA vaccines may utilise non-replicating mRNA or self-replicating RNA (also referred to as self-amplifying mRNA or SAM). Non-replicating mRNA-based vaccines typically encode an antigen of interest and contain 5′ and 3′ untranslated regions (UTRs), a 5′ cap and a poly(A) tail; whereas self-amplifying RNAs also encode viral replication machinery that enables intracellular RNA amplification (Pardi, 2018).

There remains a need for an influenza vaccine that provides a broad and robust immune response against influenza virus. There particularly remains a need for an influenza vaccine that protects individuals from heterologous strains of influenza virus (i.e. a ‘universal vaccine’), including evolving seasonal and pandemic influenza virus strains of the future.

### SUMMARY OF THE INVENTION

It has been found that the immunogenicity of the influenza HA stem region is enhanced when delivered in the form of carrier-formulated mRNA.

In particular, or in addition, it has been found the influenza HA stem polypeptides encoded by the carrier-formulated mRNAs induce a homologous, a heterologous and/or a heterosubtypic cross-reactive immunogenic responses against Influenza virus, suitably against Influenza A virus, more suitably against Influenza A virus subtypes of Group 1 and/or Group 2.

The invention therefore provides a carrier-formulated mRNA comprising at least one coding sequence encoding an influenza HA stem polypeptide. As the mRNA encodes an influenza HA stem polypeptide, there is provided a carrier-formulated mRNA encoding the stem polypeptide but not an influenza HA head region. Therefore, the mRNA does not encode a full-length influenza HA protein.

In some embodiments, the carrier is a lipid nanoparticle (LNP).

In some embodiments, the LNP comprises a PEG-modified lipid, a non-cationic lipid, a sterol, and an ionisable cationic lipid.

In some embodiments, the ionisable cationic lipid has the formula III:

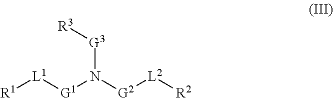

or a pharmaceutically acceptable salt, tautomer or stereoisomer thereof, wherein:
L1 or L2 is each independently —O(C═O)— or —(C═O)O—;
G1 and G2 are each independently unsubstituted C1-C12 alkylene or C1-C12 alkenylene;
G3 is C1-C24 alkylene, C1-C24 alkenylene, C3-C8 cycloalkylene, or C3-C8 cycloalkenylene;
R1 and R2 are each independently C6-C24 alkyl or C6-C24 alkenyl;
R3 is H, OR5, CN, —C(═O)OR4, —OC(═O)R4 or —NR5C(═O)R4;
R4 is C1-C12 alkyl;
R5 is H or C1-C6 alkyl.

In some embodiments, the ionisable cationic lipid has the formula III:

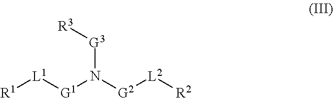

or a pharmaceutically acceptable salt, tautomer or stereoisomer thereof, wherein:
L1 or L2 is each independently —O(C═O)— or —(C═O)O—;
G1 and G2 are each independently unsubstituted C1-C12 alkylene;
G3 is C1-C24 alkylene;
R1 and R2 are each independently C6-C24 alkyl;
R3 is OR5; and
R5 is H.

In some embodiments, the ionisable cationic lipid has the formula:

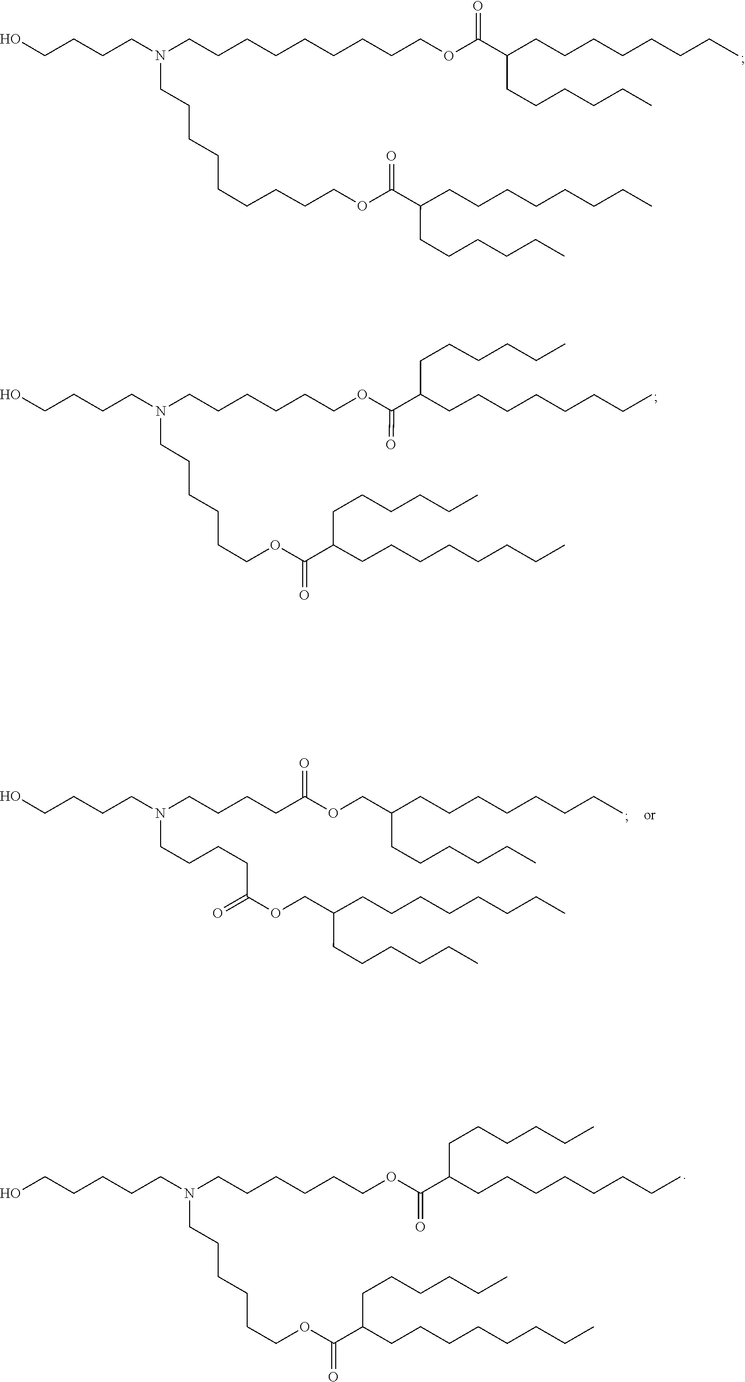 [SMILES: CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)COC(=O)CCCCN(CCCCO)CCCCC(=O)OCC(CCCCCC)CCCCCCCC ; CCCCCCCCC(CCCCCC)C(=O)OCCCCCCN(CCCCCO)CCCCCCOC(=O)C(CCCCCC)CCCCCCCC]

In some embodiments, the ionisable cationic lipid has the formula III-3:

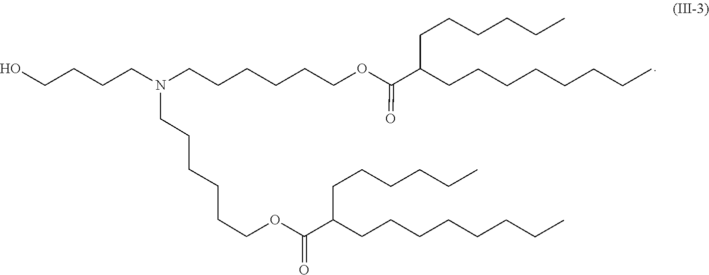 [SMILES: CCCCCCCCC(CCCCCC)C(=O)OCCCCCCN(CCCCO)CCCCCCOC(=O)C(CCCCCC)CCCCCCCC]

In some embodiments, the at least one PEG-lipid comprises PEG-DMG or PEG-cDMA.

In some embodiments, the at least one PEG-lipid comprises according to formula IVa:

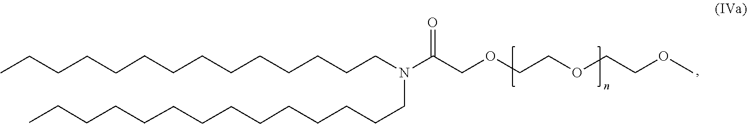

wherein n has a mean value ranging from 30 to 60, suitably wherein n has a mean value of about 45, 46, 47, 48, 49, 50, 51, 52, 53, 54, most suitably wherein n has a mean value of 49 or 45; or
wherein n is an integer selected such that the average molecular weight of the PEG lipid is about 2500 g/mol.

In some embodiments, the ionisable cationic lipid has the formula III-3:

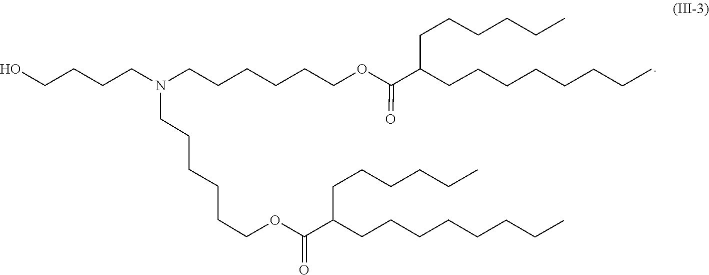 [SMILES: CCCCCCCCC(CCCCCC)C(=O)OCCCCCCN(CCCCO)CCCCCCOC(=O)C(CCCCCC)CCCCCCCC]

In some embodiments, the non-cationic lipid is a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) or sphingomyelin (SM), suitably the neutral lipid is DSPC.

In some embodiments, the sterol is cholesterol.

In some embodiments, the LNP comprise a PEG-modified lipid at around 0.5 to 15 molar %, a non-cationic lipid at around 5 to 25 molar %, a sterol at around 25 to 55 molar % and an ionisable cationic lipid at around 20 to 60 molar %.

In some embodiments, the LNP are 50 to 200 nm in diameter.

In some embodiments, the LNP have a polydispersity of 0.4 or less, such as 0.3 or less.

In some embodiments, the ratio of nucleotide (N) to phospholipid (P) is in the range of 1N:1P to 20N:1P, 1N:1P to 10N:1P, 2N:1P to 8N:1P, 2N:1P to 6N:1P or 3N:1P to 5N:1P.

In some embodiments, at least half of the mRNA is encapsulated in the LNP, suitably at least 85%, especially at least 95%, such as all of it.

In some embodiments, the mRNA comprises at least one additional coding sequence which encodes one or more heterologous peptide or protein elements selected from a signal peptide, a linker, a helper epitope, an antigen clustering element, a trimerization element, a transmembrane element, a protein nanoparticle and/or a VLP-forming sequence.

In some embodiments, the mRNA comprises at least one additional coding sequence which encodes a protein nanoparticle.

In some embodiments, the protein nanoparticle is ferritin.

In some embodiments, the ferritin is selected from bacterial and insect ferritin.

In some embodiments, the ferritin is bacterial ferritin.

In some embodiments, the bacterial ferritin is H. pylori ferritin.

In some embodiments, the protein nanoparticle and the influenza HA stem polypeptide are connected by a linker, and wherein the linker consists of 1 to 10 residues, suitably of 2 to 5 residues, for example 2, 3, 4 or 5 residues.

In some embodiments, the linker comprises or consists of the polypeptide sequence SGG.

In some embodiments, the transmembrane element is a native influenza HA transmembrane element.

In some embodiments, the signal peptide is a natural leader or an HLA-Dra leader.

In some embodiments, the mRNA comprises or consists of coding sequences encoding a signal peptide, suitably a natural leader, said at least one coding sequence, a linker and a transmembrane element.

In some embodiments, the mRNA comprises or consists of coding sequences encoding a signal peptide, suitably a natural leader, said at least one coding sequence, a linker and a protein nanoparticle, suitably bacterial ferritin, more suitably H. pylori ferritin.

In some embodiments, the influenza HA stem polypeptide is a polypeptide comprising or consisting of a full-length influenza HA stem region.

In some embodiments, the influenza HA stem polypeptide is a polypeptide comprising or consisting of an immunogenic fragment of an influenza HA stem region.

In some embodiments, the influenza HA stem polypeptide is a polypeptide comprising or consisting of an immunogenic variant of an influenza HA stem region.

In some embodiments, the influenza HA stem polypeptide is derived from influenza A, such as influenza A Group 1 or Group 2.

In some embodiments, the influenza HA stem polypeptide is derived from influenza A Group 1, suitably influenza A subtype H1, H2, H5, H6, H8, H9, H11, H12, H13, H16, H17 or H18. In some embodiments, the influenza HA stem polypeptide is derived from influenza A subtype H1.

In some embodiments, the influenza HA stem polypeptide comprises or consists of an amino acid sequence having at least 90%, 95%, 98% or 99% identity to the amino acid sequence set forth in any one of SEQ ID NO:1 or SEQ ID NO: 2. In some embodiments, the influenza HA stem polypeptide comprises or consists of an amino acid sequence having at least 90%, 95%, 98% or 99% identity to the amino acid sequence set forth in SEQ ID NO: 2

In some embodiments, the influenza HA stem polypeptide comprises or consists of the amino acid sequence set forth in any one of SEQ ID NO:1 or SEQ ID NO: 2. In some embodiments, the influenza HA stem polypeptide comprises or consists of the amino acid sequence set forth in SEQ ID NO: 2.

In some embodiments, the mRNA comprises or consists of coding sequences encoding an amino acid sequence having at least 90%, 95%, 98%, 99% or 100% identity to amino acid sequence set forth in any one of SEQ ID NO: 6 or SEQ ID NO: 7. In some embodiments, the mRNA comprises or consists of coding sequences encoding an amino acid sequence having at least 90%, 95%, 98%, 99% or 100% identity to amino acid sequence set forth in SEQ ID NO: 7 In some embodiments the mRNA comprises or consists of coding sequences encoding an amino acid sequence having at least 90%, 95%, 98%, 99% or 100% identity to amino acid sequence set forth in SEQ ID NO: 12.

In some embodiments, the mRNA comprises or consists of a nucleic acid sequence having at least 90%, 95%, 98%, 99% or 100% identity to the nucleic sequence set forth in any one of SEQ ID NO: 16 or SEQ ID NO: 17.

In some embodiments, the mRNA comprises or consists of a nucleic acid sequence having at least 90%, 95%, 98%, 99% or 100% identity to the nucleic sequence set forth in any one of SEQ ID NO: 22 or SEQ ID NO: 23.

In some embodiments, the influenza HA stem polypeptide is derived from influenza A Group 2, suitably influenza A subtype H3, H4, H7, H10, H14 and H15. In some embodiments, the influenza HA stem polypeptide is derived from influenza A subtype H3, H7 or H10.

In some embodiments, the influenza HA stem polypeptide comprises or consists of an amino acid sequence having at least 90%, 95%, 98% or 99% identity to the amino acid sequence set forth in any one of SEQ ID NO: 3, SEQ ID NO: 4 or SEQ ID NO: 10.

In some embodiments, the influenza HA stem polypeptide comprises or consists of the amino acid sequence set forth in any one of SEQ ID NO: 3, SEQ ID NO: 4 or SEQ ID NO: 10.

In some embodiments, the mRNA comprises a HA stem coding sequence having at least 90%, 95%, 98% or 99% identity to the nucleic acid sequence of SEQ ID NO: 19, SEQ ID NO: 20 or SEQ ID NO: 28.

In some embodiments, the mRNA comprises or consists of coding sequences encoding an amino acid sequence having at least 90%, 95%, 98%, 99% or 100% identity to amino acid sequence set forth in any one of SEQ ID NO: 8, SEQ ID NO: 9 or SEQ ID NO: 11.

In some embodiments, the mRNA comprises or consists of coding sequences encoding an amino acid sequence having at least 90%, 95%, 98%, 99% or 100% identity to amino acid sequence set forth in any one of SEQ ID NO: 13, SEQ ID NO: 14 or SEQ ID NO: 15.

In some embodiments, the mRNA comprises or consists of a nucleic acid sequence having at least 90%, 95%, 98%, 99% or 100% identity to the nucleic sequence set forth in any one of SEQ ID NO: 18 to 21.

In some embodiments, the mRNA comprises or consists of a nucleic acid sequence having at least 90%, 95%, 98%, 99% or 100% identity to the nucleic sequence set forth in any one of SEQ ID NO: 24 to 29.

In some embodiments, the coding sequence is a codon modified coding sequence, wherein the amino acid sequence encoded by the codon modified coding sequence is suitably not being modified compared to the amino acid sequence encoded by the corresponding wild type or reference coding sequence.

In some embodiments, the codon modified coding sequence is selected from C maximized coding sequence, CAI maximized coding sequence, human codon usage adapted coding sequence, G/C content modified coding sequence, and G/C optimized coding sequence, or any combination thereof.

In some embodiments, the codon modified coding sequence has a G/C content of at least about 45%, 50%, 55%, or 60%.

In some embodiments, the influenza HA stem polypeptide is 400 residues or fewer in length, especially 300 residues or fewer, in particular 250 residues or fewer, such as 220 residues or fewer.

In some embodiments, the influenza HA stem polypeptide is 130 residues or more in length, especially 160 residues or more, in particular 180 residues or more, such as 190 residues or more.

In some embodiments, the influenza HA stem polypeptide is 130 to 400 residues in length, especially 160 to 300, in particular 180 to 250, such as 190 to 220.

In some embodiments, the carrier-formulated mRNA comprises two or more coding sequences each encoding an influenza HA stem polypeptide, wherein said coding sequences are encoded on separate mRNA molecules.

In some embodiments, the carrier-formulated mRNA comprises two or more coding sequences each encoding an influenza HA stem polypeptide, wherein said coding sequences are encoded on the same mRNA molecule.

In some embodiments, said two or more coding sequences encode different influenza HA stem polypeptides.

In some embodiments, the two or more coding sequences comprise three or four coding sequences each encoding an influenza HA stem polypeptide.

In some embodiments, said two or more coding sequences that encode influenza HA stem polypeptides derived from influenza A, such as influenza A Group 1 and/or influenza A Group 2.

In some embodiments, at least one of said two or more coding sequence that encodes an influenza HA stem polypeptide derived from influenza A Group 1, suitably influenza A subtype H1, H2, H5, H6, H8, H9, H11, H12, H13, H16, H17 and/or H18; and at least one of said two or more coding sequence that encodes an influenza HA stem polypeptide derived from influenza A Group 2, suitably influenza A subtype H3, H4, H7, H10, H14 and/or H15.

In some embodiments, at least one of said two or more coding sequence that encodes an influenza HA stem polypeptide derived from influenza A subtype H1; and at least one of said two or more coding sequence that encodes an influenza HA stem polypeptide derived from influenza A subtype H3, H7 and/or H10.

In some embodiments, at least one of said two or more coding sequence that encodes an influenza HA stem polypeptide derived influenza A subtype H1 and at least one of said two or more coding sequence that encodes an influenza HA stem polypeptide derived from influenza A subtype H3.

In some embodiments, at least one of said two or more coding sequence that encodes an influenza HA stem polypeptide derived from influenza A subtype H1; and at least one of said two or more coding sequence that encodes an influenza HA stem polypeptide derived from influenza A subtype H10.

In some embodiments, the carrier-formulated mRNA comprises three or more coding sequences each encoding an influenza HA stem polypeptide, at least one of said three or more coding sequence that encodes an influenza HA stem polypeptide derived influenza A subtype H7.

In some embodiments, the carrier-formulated mRNA comprises at least three coding sequences each encoding an influenza HA stem polypeptide, but not comprising a coding sequence that encodes an influenza HA stem polypeptide derived influenza A subtype H10.

In some embodiments, said influenza HA stem polypeptide derived from influenza A Group 1 comprises or consists of an amino acid sequence having at least 90%, 95%, 98% or 99% identity to the amino acid sequence set forth in any one of SEQ ID NO:1 or SEQ ID NO: 2. In some embodiments, said influenza HA stem polypeptide derived from influenza A Group 1 comprises or consists of an amino acid sequence having at least 90%, 95%, 98% or 99% identity to the amino acid sequence set forth in SEQ ID NO: 2.

In some embodiments, said influenza HA stem polypeptide derived from influenza A Group 1 comprises or consists of the amino acid sequence set forth in any one of SEQ ID NO:1 or SEQ ID NO: 2. In some embodiments, said influenza HA stem polypeptide derived from influenza A Group 1 comprises or consists of the amino acid sequence set forth in SEQ ID NO: 2.

In some embodiments, the mRNA comprises or consists of a nucleic acid sequence having at least 90%, 95%, 98%, 99% or 100% identity to the nucleic sequence set forth in any one of SEQ ID NO: 16 or SEQ ID NO: 17.

In some embodiments, the mRNA comprises or consists of a nucleic acid sequence having at least 90%, 95%, 98%, 99% or 100% identity to the nucleic sequence set forth in any one of SEQ ID NO: 22 or SEQ ID NO: 23.

In some embodiments, said influenza HA stem polypeptide derived from influenza A Group 2 comprises or consists of an amino acid sequence having at least 90%, 95%, 98% or 99% identity to the amino acid sequence set forth in any one of SEQ ID NO: 3, SEQ ID NO: 4 or SEQ ID NO: 10.

In some embodiments, said influenza HA stem polypeptide derived from influenza A Group 2 comprises or consists of the amino acid sequence set forth in any one of SEQ ID NO: 3, SEQ ID NO: 4 or SEQ ID NO: 10.

In some embodiments, the mRNA comprises a HA stem coding sequence having at least 90%, 95%, 98% or 99% identity to the nucleic acid sequence of SEQ ID NO: 19, SEQ ID NO: 20 or SEQ ID NO: 28.

In some embodiments, said influenza HA stem polypeptide derived from influenza A Group 2 comprises or consists of the amino acid sequence set forth in SEQ ID NO: 3.

In some embodiments, the mRNA comprises or consists of a nucleic acid sequence having at least 90%, 95%, 98%, 99% or 100% identity to the nucleic sequence set forth in any one of SEQ ID NO: 18 to 21.

In some embodiments, the mRNA comprises or consists of a nucleic acid sequence having at least 90%, 95%, 98%, 99% or 100% identity to the nucleic sequence set forth in any one of SEQ ID NO: 24 to 29.

In some embodiments, the mRNA comprises a 5′ cap, suitably m7G, cap0, cap1, cap2, a modified cap0 or a modified cap1 structure, suitably a 5′-cap1 structure.

In some embodiments, the mRNA comprises a poly(A) tail sequence, suitably comprising 30 to 200 adenosine nucleotides and/or at least one poly(C) sequence, suitably comprising 10 to 40 cytosine nucleotides.

In some embodiments, the mRNA comprises at least one histone stem-loop.

In some embodiments, the mRNA comprises at least one poly(A) tail sequence comprising 30 to 200 adenosine nucleotides wherein the 3′ terminal nucleotide of said RNA is an adenosine. In some embodiments, the mRNA comprises at least one poly(A) tail sequence comprising 100 adenosine nucleotides wherein the 3′ terminal nucleotide of said RNA is an adenosine.

In some embodiments, the mRNA comprises a 5′ untranslated region (UTR).

In some embodiments, the 5′ UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

In some embodiments, the mRNA comprises a 3′ UTR.

In some embodiments, the 3′ UTR comprises or consists of a nucleic acid sequence derived from a 3′-UTR of a gene selected from PSMB3, ALB7, CASP1, COX6B1, GNAS, NDUFA1 and RPS9, or from a homolog, a fragment or a variant of any one of these genes.

In some embodiments, the mRNA comprises an heterologous 5′-UTR that comprises or consists of a nucleic acid sequence derived from a 5′-UTR from HSD17B4 and at least one heterologous 3′-UTR comprises or consists of a nucleic acid sequence derived from a 3′-UTR of PSMB3.

In some embodiments, the mRNA comprises from 5′ to 3′:
i) 5′-cap1 structure;
ii) 5′-UTR derived from a 5′-UTR of a HSD17B4 gene;
iii) the coding sequence;
iv) 3′-UTR derived from a 3′-UTR of a PSMB3 gene;
v) optionally, a histone stem-loop sequence; and
vi) poly(A) sequence comprising about 100 A nucleotides, wherein the 3′ terminal nucleotide of said RNA is an adenosine.

In some embodiments, the mRNA does not comprise chemically modified nucleotides.

In some embodiments, the mRNA comprises at least one chemical modification.

In some embodiments, the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In some embodiments, the chemical modification is N1-methylpseudouridine and/or pseudouridine. In some embodiments, the chemical modification is N1-methylpseudouridine.

In some embodiments, the mRNA comprises the chemical modification being a uridine modification, preferably wherein 100% of the uridine positions in the mRNA are modified.

In some embodiments, the mRNA is non-replicating.

In some embodiments, the mRNA is self-replicating.

In some embodiments, the self-replicating RNA molecule encodes (i) a RNA-dependent RNA polymerase which can transcribe RNA from the self-replicating RNA molecule and (ii) the influenza HA stem polypeptide.

In some embodiments, the RNA molecule comprises two open reading frames, the first of which encodes an alphavirus replicase and the second of which encodes the influenza HA stem polypeptide.

In some embodiments, the RNA molecule comprises three open reading frames, the first of which encodes an alphavirus replicase, the second of which encodes the influenza HA stem polypeptide and the third of which encodes a protein nanoparticle.

In some embodiments, the mRNA has the configuration 5′cap-5′UTR-non-structural proteins (NSP) 1-4-subgenomic promoter-influenza HA stem polypeptide-linker-protein nanoparticle-3′UTR-polyA.

Also provided is an immunogenic composition comprising the carrier-formulated mRNA as defined herein, wherein the composition optionally comprises at least one pharmaceutically acceptable carrier.

In some embodiments, the composition is a multivalent composition comprising a plurality or at least one further mRNA in addition to the mRNA as defined herein.

In some embodiments, the multivalent composition comprises two or more mRNA as defined herein. In some embodiments, the multivalent composition comprises two, three or four mRNA as defined herein, each encoding a different influenza HA stem polypeptide.

In some embodiments, said two or more mRNA encode influenza HA stem polypeptides derived from influenza A, such as influenza A Group 1 and/or influenza A Group 2.

In some embodiments, at least one of said two or more mRNA encodes an influenza HA stem polypeptide derived from influenza A Group 1, suitably influenza A subtype H1, H2, H5, H6, H8, H9, H11, H12, H13, H16, H17 and/or H18; and at least one of said two or more mRNA encodes an influenza HA stem polypeptide derived from influenza A Group 2, suitably influenza A subtype H3, H4, H7, H10, H14 and/or H15.

In some embodiments, at least one of said two or more mRNA encodes an influenza HA stem polypeptide derived from influenza A subtype H1; and at least one of said two or more mRNA encodes an influenza HA stem polypeptide derived from influenza A subtype H3, H7 and/or H10. In some embodiments, at least one of said two or more mRNA encodes an influenza HA stem polypeptide derived from influenza A subtype H1; and at least one of said two or more mRNA encodes an influenza HA stem polypeptide derived from influenza A subtype H3.

In some embodiments, at least one of said two or more mRNA are non-replicating. In some embodiments, each of said two or more mRNA are non-replicating.

Also provided is a vaccine comprising the mRNA as defined herein and/or the immunogenic composition as defined herein.

In some embodiments, the vaccine is a multivalent vaccine comprising a plurality or at least more than one of the RNA as defined herein, or a plurality or at least more than one of the composition as defined herein.

Also provided is a kit or kit of parts comprising the RNA as defined herein, and/or the composition as defined herein, and/or the vaccine as defined herein, optionally comprising a liquid vehicle for solubilising, and, optionally, technical instructions providing information on administration and dosage of the components.

Also provided is the carrier-formulated mRNA as defined herein, the immunogenic composition as defined herein, the vaccine as defined herein, the kit or kit of parts as defined herein for use as a medicament.

Also provided is the RNA as defined herein, the composition as defined herein, the vaccine as defined herein, the kit or kit of parts as defined herein, for use in the treatment or prophylaxis of an infection with an influenza virus, suitably an influenza A virus.

In some embodiments, a single dose of the carrier-formulated mRNA is 0.001 to 1000 μg, especially 1 to 500 μg, in particular 10 to 250 μg total mRNA.

In some embodiments, the use is for intramuscular administration.

In some embodiments, an immune response is elicited. In some embodiments, an adaptative immune response is elicited. In some embodiments, a protective adaptative immune response against an influenza virus is elicited, suitably against an influenza A virus.

In some embodiments, the elicited immune response reduces partially or completely the severity of one or more symptoms and/or time over which one or more symptoms of influenza virus infection are experienced by the subject.

In some embodiments, the elicited immune response reduces the likelihood of developing an established influenza virus infection after challenge.

In some embodiments, the elicited immune response slows progression of influenza.

Also provided is a method of treating or preventing a disorder, wherein the method comprises applying or administering to a subject in need thereof the carrier-formulated mRNA as defined herein, the composition as defined herein, the vaccine as defined herein or the kit or kit of parts as defined herein.

In some embodiments, the disorder is an infection with an influenza virus. In some embodiments, the disorder is an infection with an influenza A virus.

In some embodiments, the subject in need is a mammalian subject. In some embodiments, the subject in need is a human subject.

Also provided is a method of eliciting an immune response, wherein the method comprises applying or administering to a subject in need thereof the carrier-formulated mRNA as defined herein, the composition as defined herein, the vaccine as defined herein or the kit or kit of parts as defined herein.

In some embodiments, the immune response is an adaptative immune response. In some embodiments, the immune response is a protective adaptative immune response against an influenza virus. In some embodiments, the immune response is a protective adaptative immune response against an influenza A virus.

In some embodiments, the adaptive immune response comprises production of antibodies that bind to a HA protein that is not encoded by the carrier formulated mRNA.

In some embodiments, the immune response comprises a homologous, a heterologous and/or a heterosubtypic cross-reactive immunogenic responses against Influenza virus. In some embodiments, the immune response comprises a homologous, a heterologous and/or a heterosubtypic cross-reactive immunogenic responses against Influenza A virus. In some embodiments, the immune response comprises a homologous, a heterologous and/or a heterosubtypic cross-reactive immunogenic responses against Influenza A virus subtypes of Group 1 and/or Group 2.

In some embodiments, the subject in need is a mammalian subject. In some embodiments, the subject in need is a human subject.

Further embodiments of the invention are provided in the text below.

### BRIEF DESCRIPTION OF THE SEQUENCES

SEQ ID NO: 1: Polypeptide sequence of stabilised HA stem from A/New Caledonia/20/1999 (H1N1)
SEQ ID NO: 2: Polypeptide sequence of stabilised HA stem from A/Michigan/45/2015 (H1N1)
SEQ ID NO: 3: Polypeptide sequence of stabilised HA stem from A/Finland/486/2004 (H3N2)
SEQ ID NO: 4: Polypeptide sequence of stabilised HA stem from A/Jiangxi/IPB13/2013 (H10N8) (also referred to as “A/Jiangxi-Donghu/346/2013”)
SEQ ID NO: 5: Polypeptide sequence of H. pylori ferritin
SEQ ID NO: 6: Polypeptide sequence of H1ssF_pylori (signal peptide-stabilised HA stem from A/New Caledonia/20/1999 (H1N1)-SGG-H. pylori ferritin)
SEQ ID NO: 7: Polypeptide sequence of H1ssF_pylori (signal peptide-stabilised HA stem from A/Michigan/45/2015 (H1N1)-SGG-H. pylori ferritin)
SEQ ID NO: 8: Polypeptide sequence of H3ssF_pylori (signal peptide-stabilised HA stem from A/Finland/486/2004 (H3N2)-SGG-H. pylori ferritin)
SEQ ID NO: 9: Polypeptide sequence of H10ssF_pylori (signal peptide-stabilised HA stem from A/Jiangxi/IPB13/2013 (H10N8)-SGG-H. pylori ferritin)
SEQ ID NO: 10: Polypeptide sequence of stabilised HA stem from A/Anhui/1/2013 (H7N9)
SEQ ID NO: 11: Polypeptide sequence of H7ssF_pylori (signal peptide-stabilised HA stem from A/Anhui/1/2013 (H7N9)-SGG-H. pylori ferritin)
SEQ ID NO: 12: Polypeptide sequence of H1ssF_TM (signal peptide-stabilised HA stem from A/Michigan/45/2015 (H1N1)-SGG-transmembrane element)
SEQ ID NO: 13: Polypeptide sequence of H3ssF_TM (signal peptide-stabilised HA stem from A/Finland/486/2004 (H3N2)-SGG-transmembrane element)
SEQ ID NO: 14: Polypeptide sequence of H10ssF_TM (signal peptide-stabilised HA stem from A/Jiangxi/IPB13/2013 (H10N8)-SGG-transmembrane element)
SEQ ID NO: 15: Polypeptide sequence of H7ssF_TM (signal peptide-stabilised HA stem from A/Anhui/1/2013 (H7N9)-SGG-transmembrane element)
SEQ ID NO: 16: Nucleic acid sequence of unmodified nativeSP_H1ss_pylori from A/Michigan/45/2015 (H1N1)
SEQ ID NO: 17: Nucleic acid sequence of N1-methylpseudouridine modified nativeSP_H1ss_pylori from A/Michigan/45/2015 (H1N1)
SEQ ID NO: 18: Nucleic acid sequence of unmodified nativeSP_H3ss_pylori from A/Finland/486/2004 (H3N2)
SEQ ID NO: 19: Nucleic acid sequence of N1-methylpseudouridine modified nativeSP_H3ss_pylori from A/Finland/486/2004 (H3N2)
SEQ ID NO: 20: Nucleic acid sequence of N1-methylpseudouridine modified nativeSP_H10ss_pylori from A/Jiangxi/IPB13/2013 (H10N8)
SEQ ID NO: 21: Nucleic acid sequence of N1-methylpseudouridine modified nativeSP_H7ss_pylori from A/Anhui/1/2013 (H7N9)
SEQ ID NO: 22: Nucleic acid sequence of unmodified nativeSP_H1ss_TM from A/Michigan/45/2015 (H1N1)
SEQ ID NO: 23: Nucleic acid sequence of N1-methylpseudouridine modified nativeSP_H1ss_TM from A/Michigan/45/2015 (H1N1)
SEQ ID NO: 24: Nucleic acid sequence of unmodified nativeSP_H3ss_TM from A/Finland/486/2004 (H3N2)
SEQ ID NO: 25: Nucleic acid sequence of N1-methylpseudouridine modified nativeSP_H3ss_TM from A/Finland/486/2004 (H3N2)
SEQ ID NO: 26: Nucleic acid sequence of unmodified nativeSP_H10ss_TM from A/Jiangxi/IPB13/2013 (H10N8)
SEQ ID NO: 27: Nucleic acid sequence of N1-methylpseudouridine modified nativeSP_H10ss_TM from A/Jiangxi/IPB13/2013 (H10N8)
SEQ ID NO: 28: Nucleic acid sequence of unmodified nativeSP_H7ss_TM from A/Anhui/1/2013 (H7N9)
SEQ ID NO: 29: Nucleic acid sequence of N1-methylpseudouridine modified nativeSP_H7ss_TM from A/Anhui/1/2013 (H7N9)

### DESCRIPTION OF THE FIGURES

FIG. 1 depicts Study A: Anti-H1 stem IgG antibody titers by ELISA at 14 days post dose 2

FIG. 2 depicts Study B: Anti-H1 stem IgG antibody titers by ELISA at 14 days post dose 2

FIG. 3 depicts Study A: Anti-H1/NC/99 IgG antibody titers by ELISA at 14 days post dose 2

FIGS. 4A and 4B depict Study B: Anti-H1/NC/99 IgG antibody titers by ELISA at 14 days post dose 2

FIGS. 5A and 5B depict Study A: Anti-H1/Mich/15 IgG antibody titers by ELISA at 14 days post dose 2

FIG. 6 depicts Study B: Anti-H1/Mich/15 IgG antibody titers by ELISA at 14 days post dose 2

FIG. 7 depicts Study A: Anti-H2/Neth/99 IgG antibody titers by ELISA at 14 days post dose 2

FIG. 8 depicts Study B: Anti-H2/Neth/99 IgG antibody titers by ELISA at 14 days post dose 2

FIG. 9 depicts Study A: Anti-H9 IgG antibody titers by ELISA at 14 days post dose 2

FIG. 10 depicts Study B: Anti-H9 IgG antibody titers by ELISA at 14 days post dose 2

FIG. 11 depicts Study A: Anti-H18 IgG antibody titers by ELISA at 14 days post dose 2

FIG. 12 depicts Study B: Anti-H18 IgG antibody titers by ELISA at 14 days post dose 2

FIG. 13 depicts Study B: Anti-H3 IgG antibody titers by ELISA at 14 days post dose 2

FIG. 14 depicts Study B: Anti-H7 IgG antibody titers by ELISA at 14 days post dose 2

FIGS. 15A and 15B depict Study B: Anti-H10 IgG antibody titers by ELISA at 14 days post dose 2

FIG. 16 depicts Study A: Percentage of stem H1/Mich/2015 specific CD4+ T cell at 14 days post dose 2

FIG. 17 depicts Study B: Percentage of stem H1/Mich/2015 specific CD4+ T cell at 14 days post dose 2

FIG. 18 depicts Study A: Percentage of stem H1/Mich/2015 specific CD8+ T cell at 14 days post dose 2

FIG. 19 depicts Study B: Percentage of stem H1/Mich/2015 specific CD8+ T cell at 14 days post dose 2

FIG. 20 depicts Study B: Percentage of stem H10/Jiangxi-Donghu specific CD4+ T cell at 14 days post dose 2

FIG. 21 depicts Study B: Percentage of stem H10/Jiangxi-Donghu specific CD8+ T cell at 14 days post dose 2

FIG. 22 depicts microneutralization titers against H1/Mich/15, H1/NC/99 and H5/Vn/04 at 14 days post dose 2

FIGS. 23A and 23B depict in vitro translation of HA stem constructs FIGS. 24A and 24B depict in vitro HA-stem trimer expression in tissues culture

FIGS. 25A and 25B depict in vitro H1-stem expression after co-transfection of H1- and H3-stem mRNAs

FIG. 26 depicts in vitro detection of H3 in H3-TM/H3-F transfected cells

FIG. 27 depicts in vitro immune stimulation of H1/H3-LNPs

FIG. 28 depicts in vivo serum IFNα levels, 18 hours post prime immunization

FIGS. 29A and 29B depict in vivo T cell responses CD4⁺IFNγ⁺TNF⁺, at day 35

FIG. 30 depicts in vivo T cell responses CD8⁺IFNγ⁺TNF⁺, at day 35

FIG. 31 depicts in vivo T cell responses CD8⁺IFNγ⁺CD107⁺, at day 35

FIG. 32 depicts in vivo anti-H1 binding antibodies, at day 21

FIG. 33 depicts in vivo anti-HA IgG antibodies by Multiplexing Serology Luminex at 14 days post dose 2 (A/Michigan/45/2015)

FIG. 34 depicts in vivo anti-HA IgG antibodies by Multiplexing Serology Luminex at 14 days post dose 2 (A/Hawaii/70/2019)

FIG. 35 depicts in vivo anti-HA IgG antibodies by Multiplexing Serology Luminex at 14 days post dose 2 (A/Christchurch/16/2010)

FIG. 36 depicts in vivo anti-HA IgG antibodies by Multiplexing Serology Luminex at 14 days post dose 2 (A/California/6/09)

FIG. 37 depicts in vivo anti-HA IgG antibodies by Multiplexing Serology Luminex at 14 days post dose 2 (A/Singapore/1/57)

FIG. 38 depicts in vivo anti-HA IgG antibodies by Multiplexing Serology Luminex at 14 days post dose 2 (A/Vietnam/1203/2004)

FIG. 39 depicts in vivo anti-HA IgG antibodies by Multiplexing Serology Luminex at 14 days post dose 2 (A/Finland/486/2004)

FIG. 40 depicts in vivo anti-HA IgG antibodies by Multiplexing Serology Luminex at 14 days post dose 2 (A/Hong Kong/45/2019)

FIG. 41 depicts in vivo anti-HA IgG antibodies by Multiplexing Serology Luminex at 14 days post dose 2 (A/Perth/16/2009)

FIG. 42 depicts in vivo anti-HA IgG antibodies by Multiplexing Serology Luminex at 14 days post dose 2 (A/Beijing/47/1992)

FIG. 43 depicts in vivo anti-HA IgG antibodies by Multiplexing Serology Luminex at 14 days post dose 2 (A/Philippines/2/1982)

FIG. 44 depicts in vivo anti-HA IgG antibodies by Multiplexing Serology Luminex at 14 days post dose 2 (A/Hong Kong/1/68)

FIG. 45 depicts in vivo anti-HA IgG antibodies by Multiplexing Serology Luminex at 14 days post dose 2 (A/Shanghai/2/2013)

FIG. 46 depicts in vivo anti-HA IgG antibodies by Multiplexing Serology Luminex at 14 days post dose 2 (A/Jiangxi-Donghu/346/2013)

FIG. 47 depicts in vivo anti-H1 A/Michigan/45/2015 stem antibodies by ADCC Reporter Bioassay at 14 days post dose 2

FIGS. 48A and 48B depicts in vitro anti-H3 stem antibodies by ADCC Reporter Bioassay

FIGS. 49A and 49B depict innate immune stimulation in vitro and in vivo

FIG. 50 depicts in vivo anti-HA IgG antibodies by Multiplexing Serology Luminex at 14 days post dose 2 (A/Michigan/45/2015) (with modified nucleosides)

FIG. 51 depicts in vivo anti-HA IgG antibodies by Multiplexing Serology Luminex at 14 days post dose 2 (A/Hawaii/70/2019) (with modified nucleosides)

FIG. 52 depicts in vivo anti-HA IgG antibodies by Multiplexing Serology Luminex at 14 days post dose 2 (A/Christchurch/16/2010) (with modified nucleosides)

FIG. 53 depicts in vivo anti-HA IgG antibodies by Multiplexing Serology Luminex at 14 days post dose 2 (A/California/6/09) (with modified nucleosides)

FIG. 54 depicts in vivo anti-HA IgG antibodies by Multiplexing Serology Luminex at 14 days post dose 2 (A/Singapore/1/57) (with modified nucleosides)

FIG. 55 depicts in vivo anti-HA IgG antibodies by Multiplexing Serology Luminex at 14 days post dose 2 (A/Vietnam/1203/2004) (with modified nucleosides)

FIG. 56 depicts in vivo anti-HA IgG antibodies by Multiplexing Serology Luminex at 14 days post dose 2 (A/Finland/486/2004) (with modified nucleosides)

FIG. 57 depicts in vivo anti-HA IgG antibodies by Multiplexing Serology Luminex at 14 days post dose 2 (A/Hong Kong/45/2019) (with modified nucleosides)

FIG. 58 depicts in vivo anti-HA IgG antibodies by Multiplexing Serology Luminex at 14 days post dose 2 (A/Perth/16/2009) (with modified nucleosides)

FIG. 59 depicts in vivo anti-HA IgG antibodies by Multiplexing Serology Luminex at 14 days post dose 2 (A/Beijing/47/1992) (with modified nucleosides)

FIG. 60 depicts in vivo anti-HA IgG antibodies by Multiplexing Serology Luminex at 14 days post dose 2 (A/Philippines/2/1982) (with modified nucleosides)

FIG. 61 depicts in vivo anti-HA IgG antibodies by Multiplexing Serology Luminex at 14 days post dose 2 (A/Hong Kong/1/68) (with modified nucleosides)

FIG. 62 depicts in vivo anti-HA IgG antibodies by Multiplexing Serology Luminex at 14 days post dose 2 (A/Shanghai/2/2013) (with modified nucleosides)

FIG. 63 depicts in vivo anti-HA IgG antibodies by Multiplexing Serology Luminex at 14 days post dose 2 (A/Jiangxi-Donghu/346/2013) (with modified nucleosides)

FIG. 64 depicts in vivo anti-H1 A/Michigan/45/2015 stem antibodies by ADCC Reporter Bioassay at 14 days post dose 2 (with modified nucleosides)

FIG. 65 depicts in vitro anti-H3 A/Finland/486/2004 (H3N2) stem antibodies by ADCC Reporter Bioassay at 14 days post dose 2

FIGS. 66A and 66B depict in vivo T cell responses CD4⁺IFNγ⁺TNF⁺, at day 35 (modified nucleosides)

FIG. 67 depicts in vivo T cell responses CD8⁺IFNγ⁺TNF⁺, at day 35 (modified nucleosides)

FIG. 68 depicts in vivo T cell responses CD8⁺IFNγ⁺CD107⁺, at day 35 (modified nucleosides)

FIG. 69 depicts schematic of HA stem-H. pylori ferritin inserts

### DETAILED DESCRIPTION OF THE INVENTION

Influenza HA Stem Polypeptide

Influenza hemagglutinin (HA) is the major surface antigen of the virion and the primary target of virus neutralizing antibodies. HA is a homotrimeric surface glycoprotein, with each monomer consisting of two disulfide-linked subunits (HA1, HA2), resulting from the proteolytic cleavage products of a single HA precursor protein. The HA1 chain forms a membrane-distal globular head and a part of the membrane-proximal stem (or ‘stalk’) region. The HA2 chain represents the major component of the stem region. The head of HA mediates receptor binding while the membrane-anchored stem is the main part of membrane fusion machinery. The invention disclosed herein relates to the influenza HA stem region when isolated from the influenza HA head region. The invention disclosed herein does not relate to the influenza HA stem region when comprised within the whole influenza HA polypeptide.

An ‘influenza HA stem polypeptide’ as used herein refers to a polypeptide comprising a full-length influenza HA stem region or an immunogenic fragment or variant of an influenza HA stem region. In one embodiment the influenza HA stem polypeptide is a polypeptide comprising or consisting of a full-length influenza HA stem region or an immunogenic fragment or variant of an influenza HA stem region.

In one embodiment the influenza HA stem polypeptide is desirably 400 residues or fewer in length, especially 300 residues or fewer, in particular 250 residues or fewer, such as 220 residues or fewer. In one embodiment the influenza HA stem polypeptide is desirably 130 residues or more in length, especially 160 residues or more, in particular 180 residues or more, such as 190 residues or more. In one embodiment the influenza HA stem polypeptide is desirably 130 to 400 residues in length, especially 160 to 300, in particular 180 to 250, such as 190 to 220.

In some embodiments, the influenza HA stem polypeptide comprises an amino acid sequence having at least 90%, 95%, 98% or 99% identity to the amino acid sequence set forth in any one of SEQ ID NO:1, SEQ ID NO: 2, SEQ ID NO: 3, SEQ ID NO: 4 or SEQ ID NO: 10, In some embodiments, the influenza HA stem polypeptide comprises an amino acid sequence having at least 90%, 95%, 98% or 99% identity to the amino acid sequence set forth in any one of SEQ ID NO: 2 or SEQ ID NO: 3.

In some embodiments, the influenza HA stem polypeptide comprises an amino acid sequence having at least 90%, 95%, 98% or 99% identity to the amino acid sequence set forth in any one of SEQ ID NO: 2 or SEQ ID NO: 4.

In some embodiments, the influenza HA stem polypeptide comprises or consists of the amino acid sequence set forth in any one of SEQ ID NO: 1, SEQ ID NO: 2, SEQ ID NO: 3, SEQ ID NO: 4 or SEQ ID NO: 10.

In some embodiments, the influenza HA stem polypeptide comprises or consists of the amino acid sequence set forth in any one of SEQ ID NO: 2 or SEQ ID NO: 3.

In some embodiments, the influenza HA stem polypeptide comprises or consists of the amino acid sequence set forth in any one of SEQ ID NO: 2 or SEQ ID NO: 4.

Suitably the influenza HA stem polypeptide is derived from type A or B influenza virus. More suitably the influenza HA stem polypeptide is derived from type A influenza virus.

In one embodiment the influenza HA stem polypeptide is derived from influenza A, such as influenza A Group 1 or Group 2.

In some embodiments, the influenza HA stem polypeptide is derived from influenza A Group1 such as subtypes H1, H2, H5, H6, H8, H9, H11, H12, H13, H16, H17 or H18, more suitably H1 or H10, more suitably H1.

In some embodiments, the influenza HA stem polypeptide comprises or consists of an amino acid sequence having at least 90%, 95%, 98% or 99% identity to the amino acid sequence set forth in any one of SEQ ID NO:1 or SEQ ID NO: 2. In some embodiments, the influenza HA stem polypeptide comprises or consists of an amino acid sequence having at least 90%, 95%, 98% or 99% identity to the amino acid sequence set forth in SEQ ID NO: 2.

In some embodiments, the influenza HA stem polypeptide comprises or consists of the amino acid sequence set forth in any one of SEQ ID NO:1 or SEQ ID NO: 2. In some embodiments, the influenza HA stem polypeptide comprises or consists of the amino acid sequence set forth in SEQ ID NO: 2.

In some embodiments, the influenza HA stem polypeptide is derived from influenza A Group 2, such as subtypes H3, H4, H7, H10, H14 and H15. In some embodiments, the influenza HA stem polypeptide is derived from influenza A H3, H7 or H10. In some embodiments, the influenza HA stem polypeptide is derived from influenza A H10. In some embodiments, the influenza HA stem polypeptide is derived from influenza A H3. In some embodiments, the influenza HA stem polypeptide is derived from influenza A H7.

In some embodiments, the influenza HA stem polypeptide comprises or consists of an amino acid sequence having at least 90%, 95%, 98% or 99% identity to the amino acid sequence set forth in any one of SEQ ID NO: 3 or SEQ ID NO: 4 or SEQ ID NO: 10.

In some embodiments, the influenza HA stem polypeptide comprises or consists of the amino acid sequence set forth in any one of SEQ ID NO: 3 or SEQ ID NO: 4 or SEQ ID NO: 10.

In an alternative embodiment the influenza HA stem polypeptide is derived from influenza B. In one embodiment the isolated influenza HA stem polypeptide is not derived from influenza A HA subtype H8, such as not derived from influenza A HA H9 clade (H8, H9 and H12).

The influenza HA stem polypeptide is not a full-length influenza HA protein. The influenza HA stem polypeptide does not comprise an influenza HA head region, more suitably the influenza HA stem polypeptide does not comprise any additional regions from influenza HA.

The influenza HA stem polypeptide is also referred to herein as an ‘antigen’ or an ‘influenza stem polypeptide’ or ‘antigenic peptides or proteins’.

In some embodiments, the carrier-formulated mRNA comprises two or more coding sequences each encoding an influenza HA stem polypeptide, wherein the coding sequences are encoded on separate mRNA molecules.

In some embodiments, the carrier-formulated mRNA comprises two or more coding sequences each encoding an influenza HA stem polypeptide, wherein the coding sequences are encoded on the same mRNA molecule.

In some embodiments, the two or more coding sequences encode different influenza HA stem polypeptides.

In some embodiments, the two or more coding sequences comprise three or four coding sequences each encoding an influenza HA stem polypeptide.

According to some embodiments, the two or more coding sequences encode influenza HA stem polypeptides derived from influenza A, such as influenza A Group 1 and/or influenza A Group 2.

In some embodiments, at least one of the two or more coding sequence encodes an influenza HA stem polypeptide derived from influenza A Group 1, such as influenza A subtype H1, H2, H5, H6, H8, H9, H11, H12, H13, H16, H17 and/or H18; and at least one of the two or more coding sequence encodes an influenza HA stem polypeptide derived from influenza A Group 2, such as influenza A subtype H3, H4, H7, H10, H14 and/or H15.

In some embodiments, at least one of the two or more coding sequence encodes an influenza HA stem polypeptide derived from influenza A H1; and at least one of the two or more coding sequence encodes an influenza HA stem polypeptide derived from influenza A H3, H7 or H10.

In some embodiments, at least one of the two or more coding sequence encodes an influenza HA stem polypeptide derived from influenza A H1; and at least one of the two or more coding sequence encodes an influenza HA stem polypeptide derived from influenza A H10.

In some embodiments, at least one of the two or more coding sequence that encodes an influenza HA stem polypeptide derived influenza A subtype H1 and at least one of the two or more coding sequence that encodes an influenza HA stem polypeptide derived from influenza A subtype H3.

In some embodiments, the carrier-formulated mRNA comprises three or more coding sequences each encoding an influenza HA stem polypeptide, at least one of the three or more coding sequence that encodes an influenza HA stem polypeptide derived influenza A subtype H7.

In some embodiments, the carrier-formulated mRNA comprises at least three coding sequences each encoding an influenza HA stem polypeptide, but not comprising a coding sequence that encodes an influenza HA stem polypeptide derived influenza A subtype H10.

In some embodiments, the influenza HA stem polypeptide derived from influenza A Group 1 comprises or consists of an amino acid sequence having at least 90%, 95%, 98% or 99% identity to the amino acid sequence set forth in any one of SEQ ID NO:1 or SEQ ID NO: 2. In some embodiments, the influenza HA stem polypeptide derived from influenza A Group 1 comprises or consists of an amino acid sequence having at least 90%, 95%, 98% or 99% identity to the amino acid sequence set forth in SEQ ID NO: 2.

In some embodiments, the influenza HA stem polypeptide derived from influenza A Group 1 comprises or consists of the amino acid sequence set forth in any one of SEQ ID NO:1 or SEQ ID NO: 2. In some embodiments, the influenza HA stem polypeptide derived from influenza A Group 1 comprises or consists of the amino acid sequence set forth in SEQ ID NO: 2.

According to some embodiments, the influenza HA stem polypeptide derived from influenza A Group 2 comprises or consists of an amino acid sequence having at least 90%, 95%, 98% or 99% identity to the amino acid sequence set forth in any one of SEQ ID NO: 3, SEQ ID NO: 4 or SEQ ID NO: 10. According to some embodiments, the influenza HA stem polypeptide derived from influenza A Group 2 comprises or consists of an amino acid sequence having at least 90%, 95%, 98% or 99% identity to the amino acid sequence set forth in SEQ ID NO: 3. According to some embodiments, the influenza HA stem polypeptide derived from influenza A Group 2 comprises or consists of an amino acid sequence having at least 90%, 95%, 98% or 99% identity to the amino acid sequence set forth in SEQ ID NO: 4. According to some embodiments, the influenza HA stem polypeptide derived from influenza A Group 2 comprises or consists of an amino acid sequence having at least 90%, 95%, 98% or 99% identity to the amino acid sequence set forth in SEQ ID NO: 10.

In some embodiments, the influenza HA stem polypeptide derived from influenza A Group 2 comprises or consists of the amino acid sequence set forth in any one of SEQ ID NO: 3, SEQ ID NO: 4 or SEQ ID NO: 10. in some embodiments, the influenza HA stem polypeptide derived from influenza A Group 2 comprises or consists of the amino acid sequence set forth in SEQ ID NO: 3. in some embodiments, the influenza HA stem polypeptide derived from influenza A Group 2 comprises or consists of the amino acid sequence set forth in SEQ ID NO: 4. in some embodiments, the influenza HA stem polypeptide derived from influenza A Group 2 comprises or consists of the amino acid sequence set forth in SEQ ID NO: 10.

The influenza HA stem polypeptide may be comprised within a construct which comprises further polypeptide sequences. The further polypeptide sequences may include, for example, one or more signal peptides and/or one or more linkers and/or one or more protein nanoparticles. Accordingly, in some embodiments, the mRNA of the invention comprises at least one additional coding sequence which encodes one or more heterologous peptide or protein elements.

In some embodiments, the one or more heterologous peptide or protein element may promote or improve secretion of the encoded stem HA antigenic peptide or protein (e.g. via secretory signal sequences), promote or improve anchoring of the encoded antigenic peptide or protein of the invention in the plasma membrane (e.g. via transmembrane elements), promote or improve formation of antigen complexes (e.g. via multimerization domains or antigen clustering elements), or promote or improve virus-like particle formation (VLP forming sequence). In addition, the nucleic acid of stem HA may additionally encode peptide linker elements, self-cleaving peptides, immunologic adjuvant sequences or dendritic cell targeting sequences.

In some embodiments, the one or more heterologous peptide or protein element is selected from a signal peptide, a linker, a helper epitope, an antigen clustering element (multimerization element), a trimerization element, a transmembrane element, a protein nanoparticle and/or a VLP-forming sequence.

In embodiments, the antigenic peptide or protein comprises a heterologous signal peptide. A heterologous signal peptide may be used to improve the secretion of the encoded stem HA antigen.

In some embodiments, the mRNA of the invention comprises at least one additional coding sequence which encodes a protein nanoparticle. In some embodiments, the protein nanoparticle is ferritin. In some embodiments, the ferritin is selected from bacterial and insect ferritin. In some embodiments, the ferritin is bacterial ferritin, such as H. pylori ferritin.

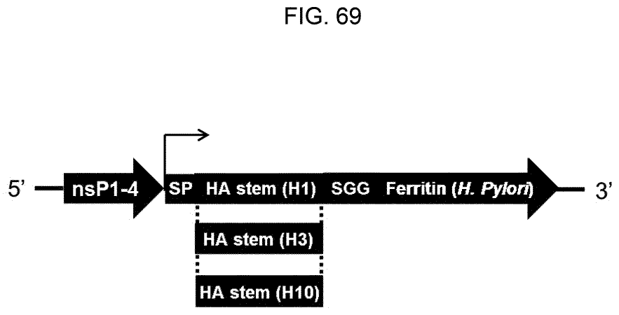

The influenza HA stem polypeptides used in some examples are comprised within a construct which includes optionally non-structural proteins 1-4 (nsP1-4), a signal peptide (SP), stabilised HA stem, a serine-glycine-glycine (SGG) linker, and H. pylori ferritin. The construct has the format: nsP1-4 (optionally)-SP-stabilised HA stem-SGG-H. pylori ferritin (FIG. 69).

The polypeptide sequences of the specific constructs used in some of the examples are SEQ ID NO: 7 (signal peptide-stabilised HA stem from A/Michigan/45/2015 (H1N1)-SGG-H. pylori ferritin), SEQ ID NO: 6 (signal peptide-stabilised HA stem from A/New Caledonia/20/1999 (H1N1)-SGG-H. pylori ferritin), SEQ ID NO: 8 (signal peptide-stabilised HA stem from A/Finland/486/2004 (H3N2)-SGG-H. pylori ferritin) and SEQ ID NO: 9 (signal peptide-stabilised HA stem from A/Jiangxi/IPB13/2013 (H10N8)-SGG-H. pylori ferritin). A further analogous construct which comprises alternatives HA stem polypeptides have the polypeptide sequence given in SEQ ID NO: 11 (signal peptide-stabilised HA stem from A/Anhui/1/2013 (H7N9)-SGG-H. pylori ferritin).

Accordingly, in one embodiment, the influenza stem polypeptide is comprised within a construct having a polypeptide sequence having 80% or greater, such as 90% or greater, such as 95% or greater, such as 98% or greater, such as 99% or greater sequence identity to any one of SEQ ID NO: 6-9 or 11. Suitably the construct comprises or consists of any one of SEQ ID NOs: 6-9 or 11.

In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 6. In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 7. In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 8. In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 9. In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 11.

In some other embodiments, the mRNA of the invention comprises at least one additional coding sequence which encodes a transmembrane element. In some embodiments, the influenza HA stem polypeptides may be comprised within a construct which includes a signal peptide, stabilised HA stem, a serine-glycine-glycine linker, and a transmembrane element.

Accordingly, in some embodiments, the influenza stem polypeptide is comprised within a construct having a polypeptide sequence having 80% or greater, such as 90% or greater, such as 95% or greater, such as 98% or greater, such as 99% or greater sequence identity to any one of SEQ ID NO: 12-15, more suitably SEQ ID NO: 12 or 13. In some embodiments, the influenza stem polypeptide is comprised within a construct having a polypeptide sequence having 80% or greater, such as 90% or greater, such as 95% or greater, such as 98% or greater, such as 99% or greater sequence identity to any one of SEQ ID NO: 12 or 13. In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 12. In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 13.

In some embodiments, the construct comprises or consists of any one of SEQ ID NOs: 12-15. In some embodiments, the construct comprises or consists of any one of SEQ ID NO: 12 or 13. In some embodiments, the construct comprises or consists of SEQ ID NO: 12. In some embodiments, the construct comprises or consists of SEQ ID NO: 13.

Suitably the immune response elicited by the influenza HA stem polypeptide produces antibodies to influenza virus. More suitably, the elicited immune response produces anti-stem region antibodies.

A Type of influenza virus refers to influenza Type A, influenza Type B or influenza type C. The designation of a virus as a specific Type relates to sequence difference in the respective M1 (matrix) protein, M2 (ion channel) protein or NP (nucleoprotein). Type A influenza viruses are further divided into Group 1 and Group 2. These Groups are further divided into subtypes, which refers to classification of a virus based on the sequence of its HA protein. Examples of current commonly recognized subtypes are H1, H2, H3, H4, H5, H6, H7, H8, H9, H10, H11, H12, H13, H14, H15, H16, H17 or H18. Group 1 influenza subtypes are H1, H2, H5, H6, H8, H9, H11, H12, H13, H16, H17 and H18. Group 2 influenza subtypes are H3, H4, H7, H10, H14 and H15. Finally, the term strain refers to viruses within a subtype that differ from one another in that they have small, genetic variations in their genome.

In one embodiment the elicited immune response produces anti-Group 1 influenza A stem region antibodies, suitably anti-H1, H2, H5, H9 and/or H18 stem region antibodies. In some embodiments, the elicited immune response produces anti-Group 2 influenza A stem region antibodies. In some embodiments, the elicited immune response produces anti-H3, H7 and/or H10. In some embodiments, the elicited immune response produces anti-H7 and/or H10 stem region antibodies. Suitably the elicited immune response produces both anti-Group 1, suitably anti-H1, H2, H5, H9 and/or H18 stem region antibodies, and anti-Group 2, suitably anti-H3, H7 and/or H10 influenza A stem region antibodies.

In some embodiments the elicited immune response produces one or more of anti-H1, H2, H3, H5, H7, H9, H10 and/or H18 stem region antibodies. More suitably the elicited immune response produces one or more of anti-H1, H2, H5, H7, H9, H10 and/or H18 stem region antibodies.

Suitably the elicited immune response produces all of anti-H1, H2, H3, H5, H7, H9, H10 and/or H18 stem region antibodies. More suitably the elicited immune response produces all of anti-H1, H2, H5, H7, H9, H10 and/or H18 stem region antibodies.

In some embodiments, the elicited immune response is homologous (against the same strain), heterologous (against different strains within a subtype) and/or heterosubtypic cross-reactive (against different strains within one or more different subtypes, e.g. from Group 1 and/or from Group 2 subtypes).

The term “homologous” in the context of an elicited immune response will be recognized and understood by the person of ordinary skill in the art, and is e.g. an immune response which is elicited against the same strain, such as the same Influenza A strain. E.g. the carrier-formulated mRNA may comprise a coding sequence encoding a stem HA polypeptide derived from A/Michigan/45/2015 (H1N1) which may elicit an immune response against A/Michigan/45/2015 (H1N1) strain.

The term “heterologous” in the context of an elicited immune response will be recognized and understood by the person of ordinary skill in the art, and is e.g. an immune response which is elicited against different strains within a subtype, such as different Influenza A strains within a subtype such as H1 or H10 subtypes. E.g. the carrier-formulated mRNA may comprise a coding sequence encoding a stem HA polypeptide derived from A/Michigan/45/2015 (H1N1) which may elicit an immune response against A/New Caledonia/20/1999 (H1N1) strain.

The term “heterosubtypic” in the context of an elicited immune response will be recognized and understood by the person of ordinary skill in the art, and is e.g. an immune response which is elicited against different strains within one or more different subtypes, e.g. from Influenza A Group 1 and/or from Group 2 subtypes. E.g. the carrier-formulated mRNA may comprise a coding sequence encoding a stem HA polypeptide derived from A/Michigan/45/2015 (H1N1) which may elicit an immune response against A/Jiangxi/IPB13/2013 (H10N8).

Full-Length Influenza HA Stem Region

In one embodiment the influenza HA stem polypeptide is a polypeptide comprising a full-length influenza HA stem region. Suitably the influenza HA stem polypeptide is a polypeptide consisting of a full-length influenza HA stem region.

The full-length influenza HA stem region is desirably 400 residues or fewer in length, especially 300 residues or fewer, in particular 250 residues or fewer, such as 220 residues or fewer. The full-length influenza HA stem region is desirably 130 residues or more in length, especially 160 residues or more, in particular 180 residues or more, such as 190 residues or more.

Suitably the full-length influenza HA stem region comprises or more suitably consists of a polypeptide sequence selected from SEQ ID NOs: 1-4 and 10. More suitably the full-length influenza HA stem region comprises or more suitably consists of SEQ ID NO: 1 or 2. More suitably the full-length influenza HA stem region comprises or more suitably consists of SEQ ID NO: 2. In some embodiments, the full-length influenza HA stem region comprises or more suitably consists of SEQ ID NO: 3, 4 or 10.

Further suitable full-length influenza HA stem regions are those disclosed in WO2013/044203, WO2015/183969 and in particular Table 2 of WO2018/045308.

Immunogenic fragments In one embodiment the influenza HA stem polypeptide is a polypeptide comprising an immunogenic fragment of an influenza HA stem region. Suitably the influenza HA stem polypeptide is a polypeptide consisting of an immunogenic fragment of an influenza HA stem region.

In some embodiments, the immunogenic fragment of an influenza HA stem region of use in the present invention comprises, such as consists of, a fragment of a full length (such as native) influenza HA stem region which is capable of eliciting neutralising antibodies and/or a T cell response (such as a CD4 or CD8 T cell response) to influenza virus, such as to influenza A virus, suitably a protective immune response (e.g. reducing partially or completely the severity of one or more symptoms and/or time over which one or more symptoms are experienced by a subject following infection, reducing the likelihood of developing an established infection after challenge and/or slowing progression of illness (e.g. extending survival)).

Suitably the immunogenic fragment of an influenza HA stem region comprises one or more epitopes from a full-length influenza HA stem region, such as one, two or three or more epitopes.

The sequence of the immunogenic fragment of an influenza HA stem region may share 80% or greater, such as 90% or greater, such as 95% or greater, such as 98% or greater, such as 99% or greater, such as most suitably 100% identity with a corresponding sequence comprised within a full length influenza HA stem region, such as the sequences provided in SEQ ID NOs: 1-4 or 10, such as SEQ ID NO: 1 or 2-4, most suitably SEQ ID NO: 2-4.

The term “fragment” as used throughout the present specification in the context of a nucleic acid sequence or an amino acid sequence may typically be a shorter portion of a full-length sequence of e.g. a nucleic acid sequence or an amino acid sequence. Accordingly, a fragment, typically, consists of a sequence that is identical to the corresponding stretch within the full-length sequence. A suitable fragment of a sequence in the context of the present invention, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived (e.g. HA stem region of an influenza virus). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore suitably refer to the entire protein or peptide as defined herein or to the entire (coding) nucleic acid molecule of such a protein or peptide. Fragments of proteins or peptides may comprise at least one epitope of those proteins or peptides.

Immunogenic Variants

In one embodiment the influenza HA stem polypeptide is a polypeptide comprising an immunogenic variant of an influenza HA stem region. Suitably the influenza HA stem polypeptide is a polypeptide consisting of an immunogenic variant of an influenza HA stem region.

In some embodiments, the immunogenic variant of an influenza HA stem region of use in the present invention comprises, such as consists of, a variant of a full length (such as native) influenza HA stem region which is capable of eliciting neutralising antibodies and/or a T cell response (such as a CD4 or CD8 T cell response) to influenza virus, such as to influenza A virus, suitably a protective immune response (e.g. reducing partially or completely the severity of one or more symptoms and/or time over which one or more symptoms are experienced by a subject following infection, reducing the likelihood of developing an established infection after challenge and/or slowing progression of illness (e.g. extending survival)).

The immunogenic variant of an influenza HA stem region may comprise, such as consist of, an amino acid sequence having at least 90%, such as at least 95%, such as at least 98%, such as at least 99%, such as 100% identity to the amino acid sequence set forth in SEQ ID NOs: 1-4 or 10, such as SEQ ID NO: 1 or 2-4, most suitably SEQ ID NO: 2-4.

Suitably the immunogenic variant of an influenza HA stem region comprises one or more epitopes from a full-length influenza HA stem region, such as one, two or three or more epitopes.

The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotides of such nucleic acid sequence.

The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). In some embodiments, these fragments and/or variants have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. In some embodiments, a variant of a protein comprises a functional variant of the protein, which means, in the context of the invention, that the variant exerts essentially the same, or at least 40%, 50%, 60%, 70%, 80%, 90% of the immunogenicity as the protein it is derived from.

Sequence Alignments

Identity or homology with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 (a standard scoring matrix; see Dayhoff, 1978) can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

Stability and Nanoparticles

For stable homotrimer assembly in its native environment, the influenza HA stem region requires the head region and the transmembrane domain. Arrangement in a homotrimer formation ensures antigenic conformational epitopes are presented. Accordingly, in one embodiment the influenza HA stem polypeptide is a stable influenza HA stem polypeptide, i.e. the polypeptide substantially retains its native conformation when expressed in a subject.

The influenza HA stem polypeptide may be synthetically stabilised (in the absence of head and transmembrane domains). Stabilisation may be achieved by helix stabilization, loop optimization, disulphide bond addition, and side-chain repacking (as disclosed in Corbett, 2019). Alternatively, or in addition, stabilisation may be achieved by providing the stem region in the form of a multimer, such as a homotrimer or a heterotrimer.

The influenza HA stem polypeptide may be provided ‘naked’ within the carrier-formulated mRNA, i.e. not bound to other stabilizing proteins or components. Alternatively, the influenza HA stem polypeptide may be co-expressed in the host with one or more other stabilizing proteins. In a particular embodiment, the influenza HA stem polypeptide is presented on the surface of nanoparticles, such as protein nanoparticles, such as those disclosed in Diaz et al 2018 including ferritin, lumazine and encapsulin.

When provided in the form of a homotrimer or a heterotrimer, the influenza HA stem polypeptide is most suitably displayed on self-assembling protein nanoparticles, such as most suitably ferritin nanoparticles, such as more suitably insect or bacterial ferritin nanoparticles.

Ferritin is a protein whose main function is intracellular iron storage. Almost all living organisms produce ferritin which is made of 24 subunits, each composed of a four-alpha-helix bundle, that self-assemble in a quaternary structure with octahedral symmetry. Its properties to self-assemble into nanoparticles are well-suited to carry and expose antigens.

In some embodiments, ferritin is used to promote the antigen clustering and may therefore promote immune responses of the encoded stem HA antigen.

According to some embodiments, the protein nanoparticles are bacterial ferritin nanoparticles. In some embodiments, the protein nanoparticles are H. pylori ferritin nanoparticles (such as those disclosed in Corbett, 2019, WO2013/044203, WO2015/183969 and WO2018/045308). When co-expressed in the host, a H. pylori ferritin linked to an influenza HA stem polypeptide will self-assembles with other H. pylori ferritins each linked to influenza HA stem polypeptides to form a nanoparticle displaying a plurality of influenza HA stem polypeptides allowing their assembly in one or more homotrimers and/or one or more heterotrimers.

Suitably the ferritin, more suitably the bacterial ferritin, still more suitably the H. pylori ferritin, and the influenza HA stem polypeptide are connected by a linker, suitably the linker consists of 1 to 10 residues, more suitably of 2 to 5 residues, such as a linker comprising the polypeptide sequence SGG, such as consisting of the polypeptide sequence SGG.

In some embodiments, the influenza HA stem polypeptide may be co-expressed in the host with a transmembrane element.

In some embodiments, the transmembrane element is a native influenza HA transmembrane element.

Additional Antigens

The present invention may involve a plurality of antigenic components, for example with the objective to elicit a broad immune response to influenza virus. Consequently, more than one antigen may be present, more than one polynucleotide encoding an antigen may be present, one polynucleotide encoding more than one antigen may be present or a mixture of antigen(s) and polynucleotide(s) encoding antigen(s) may be present. Polysaccharides such as polysaccharide conjugates may also be present.

In some embodiments, by the term antigen is meant a peptide, a protein or a polypeptide which is capable of eliciting an immune response. Suitably the antigen comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

mRNA

Messenger RNA (mRNA) can direct the cellular machinery of a subject to produce proteins. mRNA may be circular or branched, but will generally be linear. The mRNA may be circular or linear.

The terms “RNA” and “mRNA” will be recognized and understood by the person of ordinary skill in the art, and are e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA provides the nucleotide coding sequence that may be translated into an amino-acid sequence of a particular peptide or protein.

In the context of the invention, the mRNA may provide at least one coding sequence encoding an antigenic protein as defined herein that is translated into a (functional) antigen after administration (e.g. after administration to a subject, e.g. a human subject).

Accordingly, the mRNA is suitable for a vaccine of the invention.

mRNA used herein are preferably provided in purified or substantially purified form i.e. substantially free from proteins (e.g., enzymes), other nucleic acids (e.g. DNA and nucleoside phosphate monomers), and the like, generally being at least about 50% pure (by weight), and usually at least 90% pure, such as at least 95% or at least 98% pure (as described in further detail below).

mRNA may be prepared in many ways e.g. by chemical synthesis in whole or in part, by digesting longer nucleic acids using nucleases (e.g. restriction enzymes), by joining shorter nucleic acids or nucleotides (e.g. using ligases or polymerases), from genomic or cDNA libraries, etc. In particular, mRNA may be prepared enzymatically using a DNA template (as described in further detail below).

The term mRNA as used herein includes conventional mRNA or mRNA analogs, such as those containing modified backbones or modified bases (e.g. pseudouridine, or the like). mRNA, may or may not have a 5 cap (as described in further detail below).

The mRNA comprises a sequence which encodes at least one antigen. Typically, the nucleic acids of the invention will be in recombinant form, i.e. a form which does not occur in nature.

For example, the mRNA may comprise one or more heterologous nucleic acid sequences (e.g. a sequence encoding another antigen and/or a control sequence such as a promoter or an internal ribosome entry site) in addition to the sequence encoding the antigen.

In some embodiments, the carrier-formulated mRNA is an artificial nucleic acid.

The term “artificial nucleic acid” as used herein is intended to refer to a nucleic acid that does not occur naturally. In other words, an artificial nucleic acid may be understood as a non-natural nucleic acid molecule. Such nucleic acid molecules may be non-natural due to its individual sequence (e.g. G/C content modified coding sequence, UTRs) and/or due to other modifications, e.g. structural modifications of nucleotides. Typically, artificial nucleic acid may be designed and/or generated by genetic engineering to correspond to a desired artificial sequence of nucleotides. In this context, an artificial nucleic acid is a sequence that may not occur naturally, i.e. a sequence that differs from the wild type or reference sequence/the naturally occurring sequence by at least one nucleotide (via e.g. codon modification as further specified below). The term “artificial nucleic acid” is not restricted to mean “one single molecule” but is understood to comprise an ensemble of essentially identical nucleic acid molecules. Accordingly, it may relate to a plurality of essentially identical nucleic acid molecules.

Alternatively, or in addition, the sequence or chemical structure of the nucleic acid may be modified compared to a naturally-occurring sequence which encodes the antigen. The sequence of the nucleic acid molecule may be modified, e.g. to increase the efficacy of expression or replication of the nucleic acid, or to provide additional stability or resistance to degradation.

In some embodiments, the carrier-formulated mRNA is a modified and/or stabilized nucleic acid, suitably a modified and/or stabilized artificial nucleic acid.

According to some embodiments, the mRNA may thus be provided as a “stabilized artificial nucleic acid” or “stabilized coding nucleic acid” that is to say a nucleic acid showing improved resistance to in vivo degradation and/or a nucleic acid showing improved stability in vivo, and/or a nucleic acid showing improved translatability in vivo. In the following, specific suitable modifications/adaptations in this context are described which are suitably to “stabilize” the nucleic acid.

In the following, suitable modifications are described that are capable of “stabilizing” the mRNA.

mRNA may also be codon optimised. In some embodiments, the mRNA comprises at least one codon modified coding sequence. In some embodiments, the at least one coding sequence of the mRNA is a codon modified coding sequence. Suitably, the amino acid sequence encoded by the at least one codon modified coding sequence is not being modified compared to the amino acid sequence encoded by the corresponding wild type or reference coding sequence.

In some embodiments, mRNA may be codon optimised for expression in human cells. By “codon optimised” is intended modification with respect to codon usage may increase translation efficacy and/or half-life of the nucleic acid. The term “codon modified coding sequence” relates to coding sequences that differ in at least one codon (triplets of nucleotides coding for one amino acid) compared to the corresponding wild type or reference coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (cf. Table 1 of WO2020002525) to optimize/modify the coding sequence for in vivo applications as outlined herein.

In some embodiments, the at least one coding sequence of the mRNA is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof.

In some embodiments, the at least one coding sequence of the mRNA has a G/C content of at least about 45%, 50%, 55%, or 60%. In particular embodiments, the at least one coding sequence of the mRNA has a G/C content of at least about 50%, 51%, 52%, 53%, 54%, 55%, 56%, 57%, 58%, 59%, 60%, 61%, 62%, 63%, 64%, 65%, 66%, 67%, 68%, 69%, or 70%.

When transfected into mammalian host cells, the mRNA comprising a codon modified coding sequence has a stability of between 12-18 hours, or greater than 18 hours, e.g., 24, 36, 48, 60, 72, or greater than 72 hours and are capable of being expressed by the mammalian host cell (e.g. a muscle cell).

When transfected into mammalian host cells, the mRNA comprising a codon modified coding sequence is translated into protein, wherein the amount of protein is at least comparable to, or suitably at least 10% more than, or at least 20% more than, or at least 30% more than, or at least 40% more than, or at least 50% more than, or at least 100% more than, or at least 200% or more than the amount of protein obtained by a naturally occurring or wild type or reference coding sequence transfected into mammalian host cells.

In embodiments, the mRNA may be modified, wherein the C content of the at least one coding sequence may be increased, suitably maximized, compared to the C content of the corresponding wild type or reference coding sequence (herein referred to as “C maximized coding sequence”). The amino acid sequence encoded by the C maximized coding sequence of the mRNA is suitably not modified compared to the amino acid sequence encoded by the respective wild type or reference coding sequence. The generation of a C maximized nucleic acid sequences may suitably be carried out using a modification method according to WO2015/062738. In this context, the disclosure of WO2015/062738 is included herewith by reference.

In some embodiments, the mRNA may be modified, wherein the G/C content of the at least one coding sequence may be optimized compared to the G/C content of the corresponding wild type or reference coding sequence (herein referred to as “G/C content optimized coding sequence”). “Optimized” in that context refers to a coding sequence wherein the G/C content is suitably increased to the essentially highest possible G/C content. The amino acid sequence encoded by the G/C content optimized coding sequence of the mRNA is suitably not modified as compared to the amino acid sequence encoded by the respective wild type or reference coding sequence. The generation of a G/C content optimized mRNA sequence may be carried out using a method according to WO2002/098443. In this context, the disclosure of WO2002/098443 is included in its full scope in the present invention.

In some embodiments, the mRNA may be modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the mRNA is suitably modified such that the frequency of the codons encoding the same amino acid corresponds to the naturally occurring frequency of that codon according to the human codon usage. For example, in the case of the amino acid Ala, the wild type or reference coding sequence is suitably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see e.g. Table 1 of WO2020002525). Accordingly, such a procedure (as exemplified for Ala) is applied for each amino acid encoded by the coding sequence of the RNA to obtain sequences adapted to human codon usage.

In embodiments, the mRNA may be modified, wherein the G/C content of the at least one coding sequence may be modified compared to the G/C content of the corresponding wild type or reference coding sequence (herein referred to as “G/C content modified coding sequence”). In this context, the terms “G/C optimization” or “G/C content modification” relate to a nucleic acid that comprises a modified, suitably an increased number of guanosine and/or cytosine nucleotides as compared to the corresponding wild type or reference coding sequence. Such an increased number may be generated by substitution of codons containing adenosine or thymidine nucleotides by codons containing guanosine or cytosine nucleotides. Suitably, nucleic acid sequences having an increased G/C content are more stable or show a better expression than sequences having an increased A/U. The amino acid sequence encoded by the G/C content modified coding sequence of the mRNA is suitably not modified as compared to the amino acid sequence encoded by the respective wild type or reference sequence. In some embodiments, the G/C content of the coding sequence of the nucleic acid is increased by at least 10%, 20%, 30%, suitably by at least 40% compared to the G/C content of the coding sequence of the corresponding wild type or reference nucleic acid sequence.

In embodiments, the mRNA may be modified, wherein the codon adaptation index (CAI) may be increased or suitably maximised in the at least one coding sequence (herein referred to as “CAI maximized coding sequence”). In some embodiments, all codons of the wild type or reference nucleic acid sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table 1 of WO2020002525, most frequent human codons are marked with asterisks). Suitably, the mRNA comprises at least one coding sequence, wherein the codon adaptation index (CAI) of the at least one coding sequence is at least 0.5, at least 0.8, at least 0.9 or at least 0.95. In some embodiments, the codon adaptation index (CAI) of the at least one coding sequence is 1 (CAI=1). For example, in the case of the amino acid Ala, the wild type or reference coding sequence may be adapted in a way that the most frequent human codon “GCC” is always used for the amino acid. Accordingly, such a procedure (as exemplified for Ala) may be applied for each amino acid encoded by the coding sequence of the mRNA to obtain CAI maximized coding sequences.

In embodiments, the mRNA may be modified by altering the number of A and/or U nucleotides in the nucleic acid sequence with respect to the number of A and/or U nucleotides in the original nucleic acid sequence (e.g. the wild type or reference sequence). In some embodiments, such an AU alteration is performed to modify the retention time of the individual nucleic acids in a composition, to (i) allow co-purification using a HPLC method, and/or to allow analysis of the obtained nucleic acid composition. Such a method is described in detail in published PCT application WO2019092153A1. Claims 1 to 70 of WO2019092153A1 herewith incorporated by reference.

In some embodiments, the at least one coding sequence of the mRNA is a codon modified coding sequence, wherein the codon modified coding sequence is selected a G/C optimized coding sequence, a human codon usage adapted coding sequence, or a G/C modified coding sequence.

A poly A tail (e.g., of about 30 adenosine residues or more) may be attached to the 3′ end of the RNA to increase its half-life.

In some embodiments, the mRNA comprises at least one poly(N) sequence, e.g. at least one poly(A) sequence, at least one poly(U) sequence, at least one poly(C) sequence, or combinations thereof.

In some embodiments, the mRNA comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of a linear RNA (or in a circular RNA), of up to about 1000 adenosine nucleotides. In some embodiments, the poly(A) sequence is essentially homopolymeric, e.g. a poly(A) sequence of e.g. 100 adenosine nucleotides has essentially the length of 100 nucleotides. In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition the at least one nucleotide—or a stretch of nucleotides—different from an adenosine nucleotide).

The poly(A) sequence may comprise about 10 to about 500 adenosine nucleotides, about 10 to about 200 adenosine nucleotides, about 40 to about 200 adenosine nucleotides, or about 40 to about 150 adenosine nucleotides. In some embodiments, the length of the poly(A) sequence may be at least about or even more than about 10, 50, 64, 75, 100, 200, 300, 400, or 500 adenosine nucleotides.

In some embodiments, the mRNA comprises at least one poly(A) sequence comprising about 30 to about 200 adenosine nucleotides. In some embodiments, the poly(A) sequence comprises about 64 adenosine nucleotides (A64). In other some embodiments, the poly(A) sequence comprises about 100 adenosine nucleotides (A100). In other embodiments, the poly(A) sequence comprises about 150 adenosine nucleotides.

In further embodiments, the mRNA comprises at least one poly(A) sequence comprising about 100 adenosine nucleotides, wherein the poly(A) sequence is interrupted by non-adenosine nucleotides, suitably by 10 non-adenosine nucleotides (A30-N10-A70).

The poly(A) sequence as defined herein may be located directly at the 3′ terminus of the mRNA. In some embodiments, the 3′-terminal nucleotide (that is the last 3′-terminal nucleotide in the polynucleotide chain) is the 3′-terminal A nucleotide of the at least one poly(A) sequence. The term “directly located at the 3′ terminus” has to be understood as being located exactly at the 3′ terminus—in other words, the 3′ terminus of the nucleic acid consists of a poly(A) sequence terminating with an A nucleotide.

In an embodiment, the mRNA comprises a poly(A) sequence of at least 70 adenosine nucleotides, suitably consecutive at least 70 adenosine nucleotides, wherein the 3′-terminal nucleotide is an adenosine nucleotide.

In embodiments, the poly(A) sequence of the nucleic acid is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a methods and means as described in WO2016174271.

The mRNA may comprise a poly(A) sequence obtained by enzymatic polyadenylation, wherein the majority of nucleic acid molecules comprise about 100 (+/−20) to about 500 (+/−50), suitably about 250 (+/−20) adenosine nucleotides.

In embodiments, the mRNA comprises a poly(A) sequence derived from a template DNA and, optionally, additionally comprises at least one additional poly(A) sequence generated by enzymatic polyadenylation, e.g. as described in WO2016091391.

In embodiments, the mRNA comprises at least one polyadenylation signal.

In embodiments, the mRNA comprises at least one poly(C) sequence.

The term “poly(C) sequence” as used herein is intended to be a sequence of cytosine nucleotides of up to about 200 cytosine nucleotides. In embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In an embodiment, the poly(C) sequence comprises about 30 cytosine nucleotides.

In embodiments, the mRNA comprises at least one histone stem-loop (hSL) or histone stem loop structure.

The term “histone stem-loop” (abbreviated as “hSL” in e.g. the sequence listing) is intended to refer to nucleic acid sequences that form a stem-loop secondary structure predominantly found in histone mRNAs.

Histone stem-loop sequences/structures may suitably be selected from histone stem-loop sequences as disclosed in WO2012019780, the disclosure relating to histone stem-loop sequences/histone stem-loop structures incorporated herewith by reference. A histone stem-loop sequence that may be used may be derived from formulae (I) or (II) of WO2012019780.

According to a further embodiment, the mRNA comprises at least one histone stem-loop sequence derived from at least one of the specific formulae (Ia) or (Ila) of the patent application WO2012019780.

In other embodiments, the mRNA does not comprise a hsL as defined herein.

In embodiments, the mRNA comprises a 3′-terminal sequence element. The 3′-terminal sequence element comprises a poly(A) sequence and optionally a histone-stem-loop sequence.

The 5 end of the RNA may be capped. The mRNA may be modified by the addition of a 5′-cap structure, which suitably stabilizes the RNA and/or enhances expression of the encoded antigen and/or reduces the stimulation of the innate immune system (after administration to a subject).

For example, the 5 end of the RNA may be capped with a modified ribonucleotide with the structure m7G (5) ppp (5) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the mRNA molecule may be further modified by a 2′-O-Methyltransferase which results in the generation of a cap 1 structure (m7Gppp [m2′-O] N), which may further increase translation efficacy.

In embodiments, the mRNA comprises a 5′-cap structure, suitably m7G, cap0, cap1, cap2, a modified cap0or a modified cap1 structure.

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA, e.g. an mRNA. In some embodiments, the 5′-cap structure is connected via a 5′-5′-triphosphate linkage to the RNA.

5′-cap structures which may be suitable are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis or in RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008016473, WO2008157688, WO2009149253, WO2011015347, and WO2013059475). Further suitable cap analogues in that context are described in WO2017066793, WO2017066781, WO2017066791, WO2017066789, WO2017/053297, WO2017066782, WO2018075827 and WO2017066797 wherein the disclosures referring to cap analogues are incorporated herewith by reference.

In embodiments, a modified cap1 structure is generated using tri-nucleotide cap analogue as disclosed in WO2017053297, WO2017066793, WO2017066781, WO2017066791, WO2017066789, WO2017066782, WO2018075827 and WO2017066797. In particular, any cap structures derivable from the structure disclosed in claim 1-5 of WO2017053297 may be suitably used to co-transcriptionally generate a modified cap1 structure. Further, any cap structures derivable from the structure defined in claim 1 or claim 21 of WO2018075827 may be suitably used to co-transcriptionally generate a modified cap1 structure.

In embodiments, the mRNA comprises a cap1 structure.

In embodiments, the 5′-cap structure may be added co-transcriptionally using tri-nucleotide cap analogue as defined herein, suitably in an RNA in vitro transcription reaction as defined herein.

In embodiments, the cap1 structure of the mRNA is formed using co-transcriptional capping using tri-nucleotide cap analogues m7G(5′)ppp(5′)(2′OMeA)pG or m7G(5′)ppp(5′)(2′OMeG)pG.

A suitable cap1 analogues in that context is m7G(5′)ppp(5′)(2′OMeA)pG.

In other embodiments, the cap1 structure of the mRNA is formed using co-transcriptional capping using tri-nucleotide cap analogue 3′OMe-m7G(5′)ppp(5′)(2′OMeA)pG.

In other embodiments, a cap0 structure of the mRNA is formed using co-transcriptional capping using cap analogue 3′OMe-m7G(5′)ppp(5′)G.

In other embodiments, the 5′-cap structure is formed via enzymatic capping using capping enzymes (e.g. vaccinia virus capping enzymes and/or cap-dependent 2′-O methyltransferases) to generate cap0or cap1 or cap2 structures. The 5′-cap structure (cap0 or cap1) may be added using immobilized capping enzymes and/or cap-dependent 2′-O methyltransferases using methods and means disclosed in WO2016193226.

For determining the presence/absence of a cap0or a cap1 structure, a capping assays as described in published PCT application WO2015101416, in particular, as described in claims 27 to 46 of published PCT application WO2015101416 can be used. Other capping assays that may be used to determine the presence/absence of a cap0or a cap1 structure of an RNA are described in PCT/EP2018/08667, or published PCT applications WO2014152673 and WO2014152659.

In embodiments, the mRNA comprises an m7G(5′)ppp(5′)(2′OMeA) cap structure. In such embodiments, the mRNA comprises a 5′-terminal m7G cap, and an additional methylation of the ribose of the adjacent nucleotide of m7GpppN, in that case, a 2′O methylated Adenosine.

In some embodiments, about 70%, 75%, 80%, 85%, 90%, 95% of the RNA (species) comprises such a cap1 structure as determined using a capping assay.

In other embodiments, the mRNA comprises an m7G(5′)ppp(5′)(2′OMeG) cap structure. In such embodiments, the mRNA comprises a 5′-terminal m7G cap, and an additional methylation of the ribose of the adjacent nucleotide, in that case, a 2′O methylated guanosine.

In some embodiments, about 70%, 75%, 80%, 85%, 90%, 95% of the coding RNA (species) comprises such a cap1 structure as determined using a capping assay.

Accordingly, the first nucleotide of the mRNA sequence, that is, the nucleotide downstream of the m7G(5′)ppp structure, may be a 2′O methylated guanosine or a 2′O methylated adenosine.

In embodiments, the A/U (A/T) content in the environment of the ribosome binding site of the mRNA may be increased compared to the A/U (A/T) content in the environment of the ribosome binding site of its respective wild type or reference nucleic acid. This modification (an increased A/U (A/T) content around the ribosome binding site) increases the efficiency of ribosome binding to the mRNA. An effective binding of the ribosomes to the ribosome binding site in turn has the effect of an efficient translation the mRNA.

Accordingly, in some embodiments, the mRNA comprises a ribosome binding site, also referred to as “Kozak sequence”.

In some embodiments, the mRNA of the invention may comprise at least one heterologous untranslated region (UTR), e.g. a 5′ UTR and/or a 3′ UTR.

The term “untranslated region” or “UTR” or “UTR element” will be recognized and understood by the person of ordinary skill in the art, and are e.g. intended to refer to a part of a nucleic acid molecule typically located 5′ or 3′ of a coding sequence. An UTR is not translated into protein. An UTR may be part of a nucleic acid, e.g. a DNA or an RNA. An UTR may comprise elements for controlling gene expression, also called regulatory elements. Such regulatory elements may be, e.g., ribosomal binding sites, miRNA binding sites, promotor elements etc.

In embodiments, the mRNA comprises a protein-coding region (“coding sequence” or “cds”), and 5′-UTR and/or 3′-UTR. Notably, UTRs may harbor regulatory sequence elements that determine nucleic acid, e.g. RNA turnover, stability, and localization. Moreover, UTRs may harbor sequence elements that enhance translation. In medical application of nucleic acid sequences (including DNA and RNA), translation of the nucleic acid into at least one peptide or protein is of paramount importance to therapeutic efficacy. Certain combinations of 3′-UTRs and/or 5′-UTRs may enhance the expression of operably linked coding sequences encoding peptides or proteins of the invention. Nucleic acid molecules harboring the UTR combinations advantageously enable rapid and transient expression of antigenic peptides or proteins after administration to a subject, suitably after intramuscular administration. Accordingly, the mRNA comprising certain combinations of 3′-UTRs and/or 5′-UTRs as provided herein is particularly suitable for administration as a vaccine, in particular, suitable for administration into the muscle, the dermis, or the epidermis of a subject.

In some embodiments, the mRNA comprises at least one heterologous 5′-UTR and/or at least one heterologous 3′-UTR. The heterologous 5′-UTRs or 3′-UTRs may be derived from naturally occurring genes or may be synthetically engineered. In embodiments, the mRNA comprises at least one coding sequence as defined herein operably linked to at least one (heterologous) 3′-UTR and/or at least one (heterologous) 5′-UTR.

In embodiments, the mRNA comprises at least one heterologous 3′-UTR.

The term “3′-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of a nucleic acid, e.g. a DNA or an RNA, located between a coding sequence and an (optional) terminal poly(A) sequence. A 3′-UTR may comprise elements for controlling gene expression, also called regulatory elements. Such regulatory elements may be, e.g., ribosomal binding sites, miRNA binding sites etc.

In some embodiments, the mRNA comprises a 3′-UTR, which may be derivable from a gene that relates to an RNA with enhanced half-life (i.e. that provides a stable RNA).

In some embodiments, a 3′-UTR comprises one or more of a polyadenylation signal, a binding site for proteins that affect a nucleic acid stability of location in a cell, or one or more miRNA or binding sites for miRNAs.

In embodiments, the mRNA comprises at least one heterologous 3′-UTR, wherein the at least one heterologous 3′-UTR comprises a nucleic acid sequence is derived or selected from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes.

Nucleic acid sequences in that context can be derived from published PCT application WO2019077001A1, in particular, claim 9 of WO2019077001A1. The corresponding 3′-UTR sequences of claim 9 of WO2019077001A1 are herewith incorporated by reference. In some embodiments, the mRNA may comprise a 3′-UTR as described in WO2016107877, the disclosure of WO2016107877 relating to 3′-UTR sequences herewith incorporated by reference. Suitable 3′-UTRs are SEQ ID NOs: 1-24 and SEQ ID NOs: 49-318 of WO2016107877, or fragments or variants of these sequences. In other embodiments, the nucleic acid comprises a 3′-UTR as described in WO2017036580, the disclosure of WO2017036580 relating to 3′-UTR sequences herewith incorporated by reference. Suitable 3′-UTRs are SEQ ID NOs: 152-204 of WO2017036580, or fragments or variants of these sequences. In other embodiments, the nucleic acid comprises a 3′-UTR as described in WO2016022914, the disclosure of WO2016022914 relating to 3′-UTR sequences herewith incorporated by reference. Particularly suitable 3′-UTRs are nucleic acid sequences according to SEQ ID NOs: 20-36 of WO2016022914, or fragments or variants of these sequences.

In embodiments, the mRNA comprises at least one heterologous 5′-UTR.

The terms “5′-untranslated region” or “5′-UTR” or “5′-UTR element” will be recognized and understood by the person of ordinary skill in the art, and are e.g. intended to refer to a part of a nucleic acid molecule located 5′ (i.e. “upstream”) of a coding sequence and which is not translated into protein. A 5′-UTR may be part of a nucleic acid located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure (e.g. for mRNA as defined herein).

In some embodiments, the mRNA comprises a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability or RNA location in a cell, or one or more miRNA or binding sites for miRNAs.

In embodiments, the mRNA comprises at least one heterologous 5′-UTR, wherein the at least one heterologous 5′-UTR comprises a nucleic acid sequence is derived or selected from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes.

Nucleic acid sequences in that context can be selected from published PCT application WO2019077001A1, in particular, claim 9 of WO2019077001A1. The corresponding 5′-UTR sequences of claim 9 of WO2019077001A1 are herewith incorporated by reference (e.g., SEQ ID NOs: 1-20 of WO2019077001A1, or fragments or variants thereof).

In some embodiments, the nucleic acid of component A and/or component B may comprise a 5′-UTR as described in WO2013143700, the disclosure of WO2013143700 relating to 5′-UTR sequences herewith incorporated by reference. Particularly suitable 5′-UTRs are nucleic acid sequences derived from SEQ ID NOs: 1-1363, SEQ ID NO: 1395, SEQ ID NO: 1421 and SEQ ID NO: 1422 of WO2013143700, or fragments or variants of these sequences. In other embodiments, the nucleic acid comprises a 5′-UTR as described in WO2016107877, the disclosure of WO2016107877 relating to 5′-UTR sequences herewith incorporated by reference. Particularly suitable 5′-UTRs are nucleic acid sequences according to SEQ ID NOs: 25-30 and SEQ ID NOs: 319-382 of WO2016107877, or fragments or variants of these sequences. In other embodiments, the nucleic acid comprises a 5′-UTR as described in WO2017036580, the disclosure of WO2017036580 relating to 5′-UTR sequences herewith incorporated by reference. Particularly suitable 5′-UTRs are nucleic acid sequences according to SEQ ID NOs: 1-151 of WO2017036580, or fragments or variants of these sequences. In other embodiments, the nucleic acid comprises a 5′-UTR as described in WO2016022914, the disclosure of WO2016022914 relating to 5′-UTR sequences herewith incorporated by reference. Particularly suitable 5′-UTRs are nucleic acid sequences according to SEQ ID NOs: 3-19 of WO2016022914, or fragments or variants of these sequences.

In embodiments, the mRNA comprises at least one coding sequence as specified herein encoding at least one stem HA antigenic protein as defined herein, operably linked to a 3′-UTR and/or a 5′-UTR selected from the following 5′UTR/3′UTR combinations (“also referred to UTR designs”):

a-1 (HSD17B4/PSMB3), a-2 (NDUFA4/PSMB3), a-3 (SLC7A3/PSMB3), a-4 (NOSIP/PSMB3), a-5 (MP68/PSMB3), b-1 (UBQLN2/RPS9), b-2 (ASAH1/RPS9), b-3 (HSD17B4/RPS9), b-4 (HSD17B4/CASP1), b-5 (NOSIP/COX6B1), c-1 (NDUFA4/RPS9), c-2 (NOSIP/NDUFA1), c-3 (NDUFA4/COX6B1), c-4 (NDUFA4/NDUFA1), c-5 (ATP5A1/PSMB3), d-1 (Rpl31/PSMB3), d-2 (ATP5A1/CASP1), d-3 (SLC7A3/GNAS), d-4 (HSD17B4/NDUFA1), d-5 (Slc7a3/Ndufa1), e-1 (TUBB4B/RPS9), e-2 (RPL31/RPS9), e-3 (MP68/RPS9), e-4 (NOSIP/RPS9), e-5 (ATP5A1/RPS9), e-6 (ATP5A1/COX6B1), f-1 (ATP5A1/GNAS), f-2 (ATP5A1/NDUFA1), f-3 (HSD17B4/COX6B1), f-4 (HSD17B4/GNAS), f-5 (MP68/COX6B1), g-1 (MP68/NDUFA1), g-2 (NDUFA4/CASP1), g-3 (NDUFA4/GNAS), g-4 (NOSIP/CASP1), g-5 (RPL31/CASP1), h-1 (RPL31/COX6B1), h-2 (RPL31/GNAS), h-3 (RPL31/NDUFA1), h-4 (Slc7a3/CASP1), h-5 (SLC7A3/COX6B1), i-1 (SLC7A3/RPS9), i-2 (RPL32/ALB7), i-2 (RPL32/ALB7), or i-3 (alpha-globin gene).

In embodiments, the mRNA comprises at least one coding sequence as defined herein encoding at least one stem HA antigenic protein as defined herein, wherein the coding sequence is operably linked to a HSD17B4 5′-UTR and a PSMB3 3′-UTR (HSD17B4/PSMB3 (UTR design a-1)).

In further embodiments, the mRNA comprises at least one coding sequence as specified herein encoding at least one stem HA antigenic protein as defined herein, wherein the coding sequence is operably linked to a SLC7A3 5′-UTR and a PSMB3 3′-UTR (SLC7A3/PSMB3 (UTR design a-3)).

In further embodiments, the mRNA comprises at least one coding sequence as specified herein encoding at least one stem HA antigenic protein as defined herein, wherein the coding sequence is operably linked to a RPL31 5′-UTR and a RPS9 3′-UTR (RPL31/RPS9 (UTR design e-2)).

In some embodiments, the mRNA comprises at least one coding sequence as defined herein encoding at least one stem HA antigenic protein as defined herein, wherein the coding sequence is operably linked to an alpha-globin (“muag”) 3′-UTR.

In some embodiments, the mRNA of the invention comprises from 5′ to 3′:
i) 5′-cap1 structure;
ii) 5′-UTR derived from a 5′-UTR of a HSD17B4 gene;
iii) the coding sequence;
iv) 3′-UTR derived from a 3′-UTR of a PSMB3 gene;
v) optionally, a histone stem-loop sequence; and
vi) poly(A) sequence comprising about 100 A nucleotides, wherein the 3′ terminal nucleotide of the RNA is an adenosine.

According to embodiments, the mRNA is a modified RNA, wherein the modification refers to chemical modifications comprising backbone modifications as well as sugar modifications or base modifications.

A modified mRNA may comprise one or more nucleotide analogs or modified nucleotides (nucleotide analogues/modifications, e.g. backbone modifications, sugar modifications or base modifications). As used herein, “nucleotide analog” or “modified nucleotide” refers to a nucleotide that contains one or more chemical modifications (e.g., substitutions) in or on the nitrogenous base of the nucleoside (e.g. cytosine (C), thymine (T) or uracil (U)), adenine (A) or guanine (G)) and/or one or more chemical modifications in or one the phosphates of the backbone. A nucleotide analog can contain further chemical modifications in or on the sugar moiety of the nucleoside (e.g. ribose, modified ribose, six-membered sugar analog, or open-chain sugar analog), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

A backbone modification in the context of the invention is a modification, in which phosphates of the backbone of the nucleotides of the RNA are chemically modified. A sugar modification in the context of the invention is a chemical modification of the sugar of the nucleotides of the RNA. Furthermore, a base modification in the context of the invention is a chemical modification of the base moiety of the nucleotides of the RNA. In this context, nucleotide analogues or modifications are suitably selected from nucleotide analogues which are applicable for transcription and/or translation.

Modified nucleobases (chemical modifications) which can be incorporated into modified nucleosides and nucleotides and be present in the mRNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′Im (I,2′-O-dimethylinosine); m3C (3-methylcytidine); Cm (2′-O-methylcytidine); s2C (2-thiocytidine); ac4C (N4-acetylcytidine); f5C (5-fonnylcytidine); m5Cm (5,2-O-dimethylcytidine); ac4Cm (N4-acetyl-2-O-methylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-O-methyl uridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,2′-O-dimethyladenosine); rn62Am (N6,N6,0-2-trimethyladenosine); m2′7G (N2,7-dimethylguanosine); m2′2′7G (N2,N2,7-trimethylguanosine); m3Um (3,2′-O-dimethyluridine); m5D (5-methyldihydrouridine); f5Cm (5-formyl-2′-O-methylcytidine); mlGm (I,2′-0-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C₂-C₆)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

According to some embodiments, the mRNA of the present invention comprises at least one chemical modification.

In some embodiments, the nucleotide analogues/modifications which may be incorporated into a modified mRNA are selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In some embodiments, the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Particularly suitable in that context are pseudouridine (ψ), N1-methylpseudouridine (m1ψ), 5-methylcytosine, and 5-methoxyuridine, more suitably pseudouridine (ψ) and N1-methylpseudouridine (m1ψ), still more suitably N1-methylpseudouridine (m1ψ).

In some embodiments, essentially all, e.g. essentially 100% of the uracil in the coding sequence of the mRNA have a chemical modification, suitably a chemical modification is in the 5-position of the uracil.

In some embodiments, the mRNA comprises the chemical modification being a uridine modification, preferably wherein 100% of the uridine positions in the mRNA are modified.

Incorporating modified nucleotides such as e.g. pseudouridine (ψ), N1-methylpseudouridine (m1ψ), 5-methylcytosine, and/or 5-methoxyuridine into the coding sequence of the mRNA may be advantageous as unwanted innate immune responses (upon administration of the coding mRNA or the vaccine) may be adjusted or reduced (if required).

In embodiments, the mRNA comprises at least one coding sequence encoding at least one antigenic protein as defined herein, wherein the coding sequence comprises at least one modified nucleotide selected from pseudouridine (ψ) and N1-methylpseudouridine (m1ψ), suitably wherein all uracil nucleotides are replaced by pseudouridine (ψ) nucleotides and/or N1-methylpseudouridine (m1ψ) nucleotides, optionally wherein all uracil nucleotides are replaced by pseudouridine (Ψ) nucleotides and/or N1-methylpseudouridine (m1Ψ) nucleotides.

In some embodiments, the mRNA does not comprise N1-methylpseudouridine (m1Ψ) substituted positions. In further embodiments, the mRNA does not comprise pseudouridine (ψ), N1-methylpseudouridine (m1ψ), 5-methylcytosine, and 5-methoxyuridine substituted position.

In some embodiments, the chemical modification is N1-methylpseudouridine and/or pseudouridine. In some embodiments, the chemical modification is N1-methylpseudouridine

In embodiments, the mRNA of the invention comprises a coding sequence that consists only of G, C, A and U nucleotides and therefore does not comprise modified nucleotides (except of the 5′ terminal cap structure (cap0, cap1, cap2)).

The mRNA may encode more than one antigen. For example, the mRNA encoding an antigen protein may encode only the antigen or may encode additional proteins.

In embodiments, the mRNA may be monocistronic, bicistronic, or multicistronic.

The term “monocistronic” will be recognized and understood by the person of ordinary skill in the art, and is e.g. intended to refer to a nucleic acid that comprises only one coding sequence. The terms “bicistronic”, or “multicistronic” as used herein will be recognized and understood by the person of ordinary skill in the art, and are e.g. intended to refer to a nucleic acid that may comprise two (bicistronic) or more (multicistronic) coding sequences.

In embodiments, the mRNA is monocistronic.

In embodiments, the mRNA is monocistronic and the coding sequence of the mRNA encodes at least two different antigenic peptides or proteins. Accordingly, the coding sequence may encode at least two, three, four, five, six, seven, eight and more antigenic peptides or proteins, linked with or without an amino acid linker sequence, wherein the linker sequence can comprise rigid linkers, flexible linkers, cleavable linkers, or a combination thereof. Such constructs are herein referred to as “multi-antigen-constructs”.

In embodiments, the mRNA may be bicistronic or multicistronic and comprises at least two coding sequences, wherein the at least two coding sequences encode two or more different antigenic peptides or proteins as specified herein. Accordingly, the coding sequences in a bicistronic or multicistronic nucleic acid suitably encodes distinct antigenic proteins or peptides as defined herein or immunogenic fragments or immunogenic variants thereof. In some embodiments, the coding sequences in the bicistronic or multicistronic constructs may be separated by at least one IRES (internal ribosomal entry site) sequence. Thus, the term “encoding two or more antigenic peptides or proteins” may mean, without being limited thereto, that the bicistronic or multicistronic nucleic acid encodes e.g. at least two, three, four, five, six or more (suitably different) antigenic peptides or proteins of virus isolates. Alternatively, the bicistronic or multicistronic nucleic acid may encode e.g. at least two, three, four, five, six or more (suitably different) antigenic peptides or proteins derived from the same virus. In that context, suitable IRES sequences may be selected from the list of nucleic acid sequences according to SEQ ID NOs: 1566-1662 of the patent application WO2017081082, or fragments or variants of these sequences. In this context, the disclosure of WO2017081082 relating to IRES sequences is herewith incorporated by reference.

It has to be understood that, in the context of the invention, certain combinations of coding sequences may be generated by any combination of monocistronic, bicistronic and multicistronic RNA constructs and/or multi-antigen-constructs to obtain an mRNA set encoding multiple antigenic peptides or proteins as defined herein.

In embodiments, the mRNA may be prepared using any method known in the art, including chemical synthesis such as e.g. solid phase RNA synthesis, as well as in vitro methods, such as RNA in vitro transcription reactions. Accordingly, in a embodiment, the RNA is obtained by RNA in vitro transcription.

Accordingly, in embodiments, the mRNA is an in vitro transcribed RNA.

The terms “RNA in vitro transcription” or “in vitro transcription” relate to a process wherein RNA is synthesized in a cell-free system (in vitro). RNA may be obtained by DNA-dependent in vitro transcription of an appropriate DNA template, which may be a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases. In a embodiment of the present invention the DNA template is linearized with a suitable restriction enzyme, before it is subjected to RNA in vitro transcription.

Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein; optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising TRIS-Citrate as disclosed in WO2017109161.

In embodiments, the cap1 structure of the mRNA is formed using co-transcriptional capping using tri-nucleotide cap analogues m7G(5′)ppp(5′)(2′OMeA)pG or m7G(5′)ppp(5′)(2′OMeG)pG.

A suitable cap1 analogue that may be used in manufacturing the coding RNA of the invention is m7G(5′)ppp(5′)(2′OMeA)pG.

In other embodiments, the cap1 structure of the mRNA is formed using co-transcriptional capping using tri-nucleotide cap analogue 3′OMe-m7G(5′)ppp(5′)(2′OMeA)pG.

In other embodiments, a cap0structure of the mRNA is formed using co-transcriptional capping using cap analogue 3′OMe-m7G(5′)ppp(5′)G.

In embodiments, the nucleotide mixture used in RNA in vitro transcription may additionally comprise modified nucleotides as defined herein. In that context, suitable modified nucleotides may be selected from pseudouridine (ψ), N1-methylpseudouridine (m1ψ), 5-methylcytosine, and 5-methoxyuridine. In embodiments, uracil nucleotides in the nucleotide mixture are replaced (either partially or completely) by pseudouridine (ψ) and/or N1-methylpseudouridine (m1ψ) to obtain a modified RNA.

In some other embodiments, the nucleotide mixture used in RNA in vitro transcription does not comprise modified nucleotides as defined herein. In embodiments, the nucleotide mixture used in RNA in vitro transcription does only comprise G, C, A and U nucleotides, and, optionally, a cap analog as defined herein.

In embodiments, the nucleotide mixture (i.e. the fraction of each nucleotide in the mixture) used for RNA in vitro transcription reactions may be optimized for the given RNA sequence, suitably as described in WO2015188933.

In this context, the in vitro transcription has been performed in the presence of a sequence optimized nucleotide mixture and optionally a cap analog.

In this context a sequence-optimized nucleoside triphosphate (NTP) mix is a mixture of nucleoside triphosphates (NTPs) for use in an in vitro transcription reaction of an RNA molecule of a given sequence comprising the four nucleoside triphosphates (NTPs) GTP, ATP, CTP and UTP, wherein the fraction of each of the four nucleoside triphosphates (NTPs) in the sequence-optimized nucleoside triphosphate (NTP) mix corresponds to the fraction of the respective nucleotide in the RNA molecule. If a ribonucleotide is not present in the RNA molecule, the corresponding nucleoside triphosphate is also not present in the sequence-optimized nucleoside triphosphate (NTP) mix.

In embodiments where more than one different RNA as defined herein have to be produced, e.g. where 2, 3, 4, 5, 6, 7, 8, 9, 10 or even more different RNAs have to be produced, procedures as described in WO2017109134 may suitably be used.

In the context of nucleic acid-based vaccine production, it may be required to provide GMP-grade nucleic acid, e.g. a GMP grade RNA or DNA. GMP-grade RNA or DNA may be produced using a manufacturing process approved by regulatory authorities. Accordingly, in some embodiments, RNA production is performed under current good manufacturing practice (GMP), implementing various quality control steps on DNA and RNA level, suitably according to WO2016180430. In embodiments, the mRNA of the invention is a GMP-grade mRNA. Accordingly, an RNA for a vaccine is suitably a GMP grade RNA.

The obtained RNA products may be purified using PureMessenger® (CureVac, TQbingen, Germany; RP-HPLC according to WO2008077592) and/or tangential flow filtration (as described in WO2016193206) and/or oligo d(T) purification (see WO2016180430).

In some embodiments, the mRNA is purified using RP-HPLC, suitably using Reversed-Phase High pressure liquid chromatography (RP-HPLC) with a macroporous styrene/divinylbenzene column (e.g. particle size 30 μm, pore size 4000 Å and additionally using a filter cassette with a cellulose based membrane with a molecular weight cutoff of about 100 kDa.

In a further embodiment, the mRNA, is lyophilized (e.g. according to WO2016165831 or WO2011069586) to yield a temperature stable dried mRNA (powder). The mRNA of the invention may also be dried using spray-drying or spray-freeze drying (e.g. according to WO2016184575 or WO2016184576) to yield a temperature stable mRNA (powder) as defined herein. Accordingly, in the context of manufacturing and purifying RNA, the disclosures of WO2017109161, WO2015188933, WO2016180430, WO2008077592, WO2016193206, WO2016165831, WO2011069586, WO2016184575, and WO2016184576 are incorporated herewith by reference.

Accordingly, in embodiments, the mRNA is a dried mRNA.

The term “dried mRNA” as used herein has to be understood as mRNA that has been lyophilized, or spray-dried, or spray-freeze dried as defined above to obtain a temperature stable dried mRNA (powder).

In embodiments, the mRNA of the invention is a purified mRNA.

The term “purified mRNA” as used herein has to be understood as RNA which has a higher purity after certain purification steps (e.g. HPLC, TFF, Oligo d(T) purification, precipitation steps) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences etc.), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full-length RNA transcripts is as close as possible to 100%. Accordingly, “purified RNA” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing the by-products. Alternatively, the degree of purity may for example be determined by an analytical agarose gel electrophoresis or capillary gel electrophoresis.

It has to be understood that “dried mRNA” as defined herein and “purified mRNA” as defined herein or “GMP-grade RNA” as defined herein may have superior stability characteristics (in vitro, in vivo) and improved efficiency (e.g. better translatability of the mRNA in vivo) and are therefore particularly suitable for a medical purpose, e.g. a vaccine.

In embodiments, the mRNA has been purified by RP-HPLC and/or TFF to remove double-stranded RNA, non-capped RNA and/or RNA fragments.

The formation of double stranded RNA as side products during e.g. RNA in vitro transcription can lead to an induction of the innate immune response, particularly IFNalpha which is the main factor of inducing fever in vaccinated subjects, which is of course an unwanted side effect. Current techniques for immunoblotting of dsRNA (via dot Blot, serological specific electron microscopy (SSEM) or ELISA for example) are used for detecting and sizing dsRNA species from a mixture of nucleic acids.

In some embodiments, the mRNA has been purified by RP-HPLC and/or TFF as described herein to reduce the amount of dsRNA.

In embodiments, the mRNA comprises about 5%, 10%, or 20% less double stranded RNA side products as an mRNA that has not been purified with RP-HPLC and/or TFF.

In some embodiments, the RP-HPLC and/or TFF purified mRNA comprises about 5%, 10%, or 20% less double stranded RNA side products as an RNA that has been purified with Oligo dT purification, precipitation, filtration and/or AEX.

In embodiments, mRNA of a composition has an RNA integrity ranging from about 40% to about 100%.

The term “RNA integrity” generally describes whether the complete RNA sequence is present in the composition. Low RNA integrity could be due to, amongst others, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, the functionality of the RNA.

The skilled person can choose from a variety of different chromatographic or electrophoretic methods for determining an RNA integrity. Chromatographic and electrophoretic methods are well-known in the art. In case chromatography is used (e.g. RP-HPLC), the analysis of the integrity of the RNA may be based on determining the peak area (or “area under the peak”) of the full length RNA in a corresponding chromatogram. The peak area may be determined by any suitable software which evaluates the signals of the detector system. The process of determining the peak area is also referred to as integration. The peak area representing the full length RNA is typically set in relation to the peak area of the total RNA in a respective sample. The RNA integrity may be expressed in % RNA integrity.

In the context of aspects of the invention, RNA integrity may be determined using analytical (RP)HPLC. Typically, a test sample of the composition comprising lipid based carrier encapsulating RNA may be treated with a detergent (e.g. about 2% Triton X100) to dissociate the lipid based carrier and to release the encapsulated RNA. The released RNA may be captured using suitable binding compounds, e.g. Agencourt AMPure XP beads (Beckman Coulter, Brea, CA, USA) essentially according to the manufacturer's instructions. Following preparation of the RNA sample, analytical (RP)HPLC may be performed to determine the integrity of RNA. Typically, for determining RNA integrity, the RNA samples may be diluted to a concentration of 0.1 g/l using e.g. water for injection (WFI). About 10 μl of the diluted RNA sample may be injected into an HPLC column (e.g. a monolithic poly(styrene-divinylbenzene) matrix). Analytical (RP)HPLC may be performed using standard conditions, for example: Gradient 1: Buffer A (0.1M TEAA (pH 7.0)); Buffer B (0.1M TEAA (pH 7.0) containing 25% acetonitrile). Starting at 30% buffer B the gradient extended to 32% buffer B in 2 min, followed by an extension to 55% buffer B over 15 minutes at a flow rate of 1 ml/min. HPLC chromatograms are typically recorded at a wavelength of 260 nm. The obtained chromatograms may be evaluated using a software and the relative peak area may be determined in percent (%) as commonly known in the art. The relative peak area indicates the amount of RNA that has 100% RNA integrity. Since the amount of the RNA injected into the HPLC is typically known, the analysis of the relative peak area provides information on the integrity of the RNA. Thus, if e.g. 100ng RNA have been injected in total, and 100ng are determined as the relative peak area, the RNA integrity would be 100%. If, for example, the relative peak area would correspond to 80ng, the RNA integrity would be 80%. Accordingly, RNA integrity in the context of the invention is determined using analytical HPLC, suitably analytical RP-HPLC.

In embodiments, mRNA of a composition has an RNA integrity ranging from about 40% to about 100%. In embodiments, the mRNA has an RNA integrity ranging from about 50% to about 100%. In embodiments, the mRNA has an RNA integrity ranging from about 60% to about 100%. In embodiments, the mRNA has an RNA integrity ranging from about 70% to about 100%. In embodiments, the mRNA integrity is for example about 50%, about 60%, about 70%, about 80%, or about 90%. RNA integrity is suitably determined using analytical HPLC, suitably analytical RP-HPLC.

In embodiments, the RNA of a composition has an RNA integrity of at least about 50%, suitably of at least about 60%, more suitably of at least about 70%, most suitably of at least about 80% or about 90%. RNA integrity is suitably determined using analytical HPLC, more suitably analytical RP-HPLC.

Following co-transcriptional capping as defined herein, and following purification as defined herein, the capping degree of the obtained RNA may be determined using capping assays as described in published PCT application WO2015101416, in particular, as described in Claims 27 to 46 of published PCT application WO2015101416 can be used. Alternatively, a capping assay described in PCT/EP2018/08667 may be used.

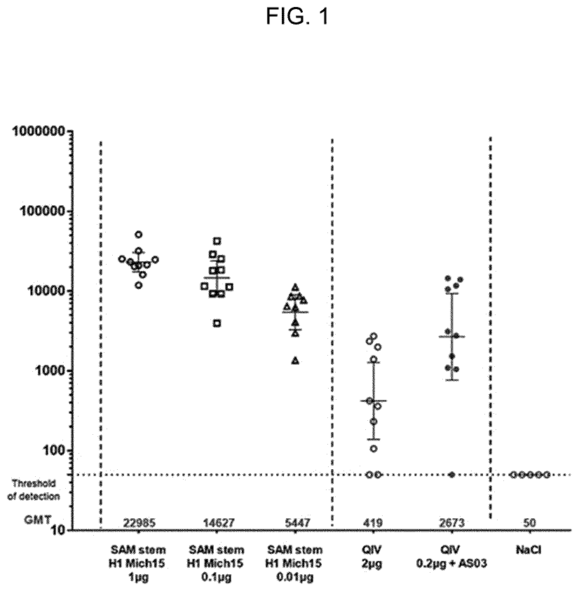

In embodiments, an automated device for performing RNA in vitro transcription may be used to produce and purify the mRNA od the invention. Such a device may also be used to produce the composition or the vaccine (as described in further detail below). In some embodiments, a device as described in WO2020002598, in particular, a device as described in claims 1 to 59 and/or 68 to 76 of WO2020002598 (and FIG. 1-18) may suitably be used.

The methods described herein may applied to a method of producing an immunogenic composition or a vaccine as described in further detail below.

In various embodiments the mRNA comprises, suitably in 5′- to 3′-direction, the following elements:
A) 5′-cap structure, suitably as specified herein;
B) 5′-terminal start element, suitably as specified herein;
C) optionally, a 5′-UTR, suitably as specified herein;
D) a ribosome binding site, suitably as specified herein;
E) at least one coding sequence, suitably as specified herein;
F) 3′-UTR, suitably as specified herein;
G) optionally, poly(A) sequence, suitably as specified herein;
H) optionally, poly(C) sequence, suitably as specified herein;
I) optionally, histone stem-loop suitably as specified herein;
J) optionally, 3′-terminal sequence element, suitably as specified herein.

According to some embodiment, the mRNA may be non-replicating.

In some embodiments, the mRNA does not comprise a replicase element (e.g. a nucleic acid encoding a replicase).

In some embodiments, the mRNA comprises or consists of a nucleic acid sequence having at least 90%, 95%, 98%, 99% or 100% identity to the nucleic sequence set forth in any one of SEQ ID NO: 16 or SEQ ID NO: 17.

In some embodiments, the mRNA comprises or consists of a nucleic acid sequence having at least 90%, 95%, 98%, 99% or 100% identity to the nucleic sequence set forth in any one of SEQ ID NO: 22 or SEQ ID NO: 23.

In some embodiments, the mRNA comprises or consists of a nucleic acid sequence having at least 90%, 95%, 98%, 99% or 100% identity to the nucleic sequence set forth in any one of SEQ ID NO: 18 to 21.

In some embodiments, the mRNA comprises or consists of a nucleic acid sequence having at least 90%, 95%, 98%, 99% or 100% identity to the nucleic sequence set forth in any one of SEQ ID NO: 24 to 29.

According to some other embodiments, the mRNA is replicating, also known as self-amplifying (SAM). A self-amplifying mRNA molecule may be an alphavirus-derived mRNA replicon. mRNA amplification can also be achieved by the provision of a non-replicating mRNA encoding an antigen in conjunction with a separate mRNA encoding replication machinery.

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see the following reference: WO2005/113782.

In certain embodiments, the self-replicating RNA molecule described herein encodes (i) a RNA-dependent RNA polymerase which can transcribe RNA from the self-replicating RNA molecule and (ii) an antigen, e.g. the influenza HA stem polypeptide. The polymerase can be an alphavirus replicase e.g. comprising one or more of alphavirus proteins nsPI, nsP2, nsP3 and nsP4 (wherein nsP stands for non-structural protein).

Whereas natural alphavirus genomes encode structural virion proteins in addition to the non-structural replicase polyprotein, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins.

Thus, a self-replicating RNA molecule useful with the invention may have two open reading frames. The first (5) open reading frame encodes a replicase, suitably an alphavirus replicase; the second (3′) open reading frame encodes an antigen, e.g. the influenza HA stem polypeptide. In some embodiments the RNA may have additional (e.g. downstream) open reading frames e.g. to encode further antigens or to encode accessory polypeptides. In some embodiments, the RNA molecule comprises three open reading frames, the first of which encodes an alphavirus replicase, the second of which encodes the influenza HA stem polypeptide and the third of which encodes a protein nanoparticle.

In certain embodiments, the self-replicating RNA molecule disclosed herein has a 5 cap (e.g. a 7-methylguanosine). This cap can enhance in vivo translation of the RNA. In some embodiments the 5′ sequence of the self-replicating RNA molecule must be selected to ensure compatibility with the encoded replicase.

A self-replicating RNA molecule may have a 3′ poly-A tail. It may also include a poly-A polymerase recognition sequence (e.g. AAUAAA) near its 3′ end.

Self-replicating RNA molecules can have various lengths, but they are typically 5000-25000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

In another embodiment, a self-replicating RNA may comprise two separate RNA molecules, each comprising a nucleotide sequence derived from an alphavirus: one RNA molecule comprises a RNA construct for expressing alphavirus replicase, and one RNA molecule comprises a RNA replicon that can be replicated by the replicase in trans. The RNA construct for expressing alphavirus replicase comprises a 5′-cap. See WO2017/162265.

The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. An RNA used with the invention ideally includes only phosphodiester linkages between nucleosides, but in some embodiments, it can contain phosphoramidate, and/or methylphosphonate linkages.

The self-replicating RNA molecule may encode a single heterologous polypeptide antigen (i.e. the antigen) or, optionally, two or more heterologous polypeptide antigens linked together in a way that each of the sequences retains its identity (e.g., linked in series) when expressed as an amino acid sequence. The heterologous polypeptides generated from the self-replicating RNA may then be produced as a fusion polypeptide or engineered in such a manner to result in separate polypeptide or peptide sequences.

The self-replicating RNA molecules described herein may be engineered to express multiple nucleotide sequences, from two or more open reading frames, thereby allowing co-expression of proteins, such as one, two or more antigens (e.g. one, two or more stem proteins) together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes an antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode an antigen can also be tested for ability to induce humoral immune responses, as evidenced, for example, by induction of B cell production of antibodies specific for the antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

In one embodiment the mRNA has the configuration 5′cap-5′UTR-non-structural proteins (NSP) 1-4-signal peptide-influenza HA stem polypeptide-linker-protein nanoparticle-3′UTR-polyA.

A non-replicating mRNA will typically contain 10000 bases or fewer, especially 8000 bases or fewer, in particular 5000 base or fewer, especially 2500 bases or fewer. A replicating mRNA will typically contain 25000 bases or fewer, especially 20000 bases or fewer, in particular 15000 bases or fewer.

A single dose of mRNA may be 0.001 to 1000 ug, 0.01 to 1000 ug, especially 1 to 500 ug, in particular 10 to 250 ug of total mRNA. A single dose of mRNA may be 0.01 to 1 ug, especially 0.05 to 0.5 ug, in particular about 0.1 ug. A single dose of mRNA may be 0.1 to 10 ug, especially 0.5 to 5 ug, in particular about 1 ug. A single dose of mRNA may be 1 to 20 ug, especially 5 to 15 ug, in particular about 10 ug.

In one embodiment the mRNA is non-replicating mRNA. In a second embodiment the mRNA is replicating mRNA.

Carriers

A range of carrier systems have been described which encapsulate or complex mRNA in order to facilitate mRNA delivery and consequent expression of encoded antigens as compared to mRNA which is not encapsulated or complexed. The present invention may utilise any suitable carrier system. Particular carrier systems of note are further described below.

In embodiments, the mRNA of the invention is complexed, encapsulated, partially encapsulated, or associated with one or more lipids (e.g. cationic lipids and/or neutral lipids), thereby forming lipid-based carriers such as liposomes, lipid nanoparticles (LNPs), lipoplexes, and/or nanoliposomes, suitably lipid nanoparticles.

In some embodiments, the two or more mRNA are formulated separately (in any formulation or complexation agent defined herein), suitably wherein the two or more mRNA are formulated in separate liposomes, lipid nanoparticles (LNP), lipoplexes, and/or nanoliposomes.

In embodiments, the two or more mRNA are co-formulated (in any formulation or complexation agent defined herein), suitably wherein the two or more mRNA are formulated in separate liposomes, lipid nanoparticles (LNP), lipoplexes, and/or nanoliposomes.

LNP

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP).

Lipid nanoparticles (LNPs) are non-virion liposome particles in which mRNA can be encapsulated. The incorporation of a nucleic acid into LNPs is also referred to herein as “encapsulation” wherein the nucleic acid, e.g. the RNA is entirely contained within the interior space of the liposomes, lipid nanoparticles (LNPs), lipoplexes, and/or nanoliposomes.

LNP delivery systems and methods for their preparation are known in the art.

The particles can include some external mRNA (e.g. on the surface of the particles), but desirably at least half of the RNA (and suitably at least 85%, especially at least 95%, such as all of it) is encapsulated.

LNPs are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the mRNA to a target tissue.

Accordingly, in embodiments, the mRNA of the invention is complexed with one or more lipids thereby forming lipid nanoparticles (LNP), liposomes, nanoliposomes, lipoplexes, suitably LNPs. In some embodiments, LNPs are suitable for intramuscular and/or intradermal administration.

In embodiments, at least about 80%, 85%, 90%, 95% of lipid-based carriers, suitably the LNPs, have a spherical morphology, suitably comprising a solid core or partially solid core.

LNPs typically comprise a cationic lipid and one or more excipients selected from neutral lipids, charged lipids, steroids and polymer conjugated lipids (e.g. PEGylated lipid). The mRNA may be encapsulated in the lipid portion of the LNP or an aqueous space enveloped by some or the entire lipid portion of the LNP. The mRNA or a portion thereof may also be associated and complexed with the LNP. An LNP may comprise any lipid capable of forming a particle to which the nucleic acids are attached, or in which the one or more nucleic acids are encapsulated. In some embodiments, the LNP comprising nucleic acids comprises one or more cationic lipids, and one or more stabilizing lipids. Stabilizing lipids include neutral lipids and PEGylated lipids.

LNP can, for example, be formed of a mixture of (i) a PEG-modified lipid (ii) a non-cationic lipid (iii) a sterol (iv) an ionisable cationic lipid. Alternatively, LNP can for example be formed of a mixture of (i) a PEG-modified lipid (ii) a non-cationic lipid (iii) a sterol (iv) a non-ionisable cationic lipid.

In some embodiments, the LNP (or liposomes, nanoliposomes, lipoplexes) comprises
(i) at least one cationic lipid;
(ii) at least one neutral lipid;
(iii) at least one steroid or steroid analogue, suitably cholesterol; and
(iv) at least one polymer conjugated lipid, suitably a PEG-lipid;
wherein (i) to (iv) are in a molar ratio of about 20-60% cationic lipid, 5-25% neutral lipid, 25-55% sterol, and 0.5-15% polymer conjugated lipid.

In vivo characteristics and behavior of LNPs can be modified by addition of a hydrophilic polymer coating, e.g. polyethylene glycol (PEG), to the LNP surface to confer steric stabilization. Furthermore, LNPs (or liposomes, nanoliposomes, lipoplexes) can be used for specific targeting by attaching ligands (e.g. antibodies, peptides, and carbohydrates) to its surface or to the terminal end of the attached PEG chains (e.g. via PEGylated lipids or PEGylated cholesterol).

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

A polymer conjugated lipid as defined herein, e.g. a PEG-lipid, may serve as an aggregation reducing lipid.

In certain embodiments, the LNP comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA).

In someembodiments, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as ω-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In embodiments, the PEGylated lipid is suitably derived from formula (IV) of published PCT patent application WO2018078053A1. Accordingly, PEGylated lipids derived from formula (IV) of published PCT patent application WO2018078053A1, and the respective disclosure relating thereto, are herewith incorporated by reference.

In some embodiments, the mRNA is complexed with one or more lipids thereby forming LNPs, wherein the LNP comprises a polymer conjugated lipid, suitably a PEGylated lipid, wherein the PEG lipid is suitably derived from formula (IVa) of published PCT patent application WO2018078053A1. Accordingly, PEGylated lipid derived from formula (IVa) of published PCT patent application WO2018078053A1, and the respective disclosure relating thereto, is herewith incorporated by reference.

In an embodiment, the mRNA is complexed with one or more lipids thereby forming lipid nanoparticles, wherein the LNP (or liposomes, nanoliposomes, lipoplexes) comprises a polymer conjugated lipid, suitably a PEGylated lipid/PEG lipid.

In some embodiments, the PEG lipid or PEGylated lipid is of formula (IVa):

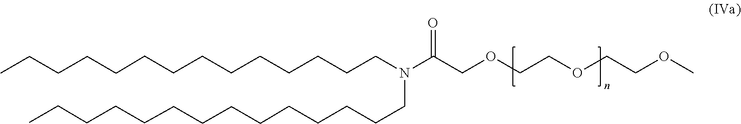

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In an embodiment n is about 49. In another embodiment n is about 45. In further embodiments, the PEG lipid is of formula (IVa) wherein n is an integer selected such that the average molecular weight of the PEG lipid is about 2000 g/mol to about 3000 g/mol or about 2300 g/mol to about 2700 g/mol, suitably about 2500 g/mol.

The lipid of formula Va as suitably used herein has the chemical term 2[(polyethylene glycol)-2000]-N,N-ditetradecylacetamide, also referred to as ALC-0159.

Further examples of PEG-lipids suitable in that context are provided in US20150376115A1 and WO2015199952, each of which is incorporated by reference in its entirety.

The PEG-modified lipid may comprise a PEG molecule with a molecular weight of 10000 Da or less, especially 5000 Da or less, in particular 3000 Da, such 2000 Da or less. Examples of PEG-modifed lipids include PEG-distearoyl glycerol, PEG-dipalmitoyl glycerol and PEG-dimyristoyl glycerol. The PEG-modified lipid is typically present at around 0.5 to 15 molar %.

In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2,5%, about 2%, about 1.5%, about 1%, about 0.5%, or about 0.3% on a molar basis (based on 100% total moles of lipids in the LNP). In embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most suitably 1.7% (based on 100% total moles of lipids in the LNP).

In various embodiments, the molar ratio of the cationic lipid to the PEGylated lipid ranges from about 100:1 to about 25:1.

In embodiments, the LNP comprises one or more additional lipids, which stabilize the formation of particles during their formulation or during the manufacturing process (e.g. neutral lipid and/or one or more steroid or steroid analogue).

In embodiments, the mRNA is complexed with one or more lipids thereby forming lipid nanoparticles, wherein the LNP comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

The non-cationic lipid may be a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) and sphingomyelin (SM). The non-cationic lipid is typically present at around 5 to 25 molar %.

In embodiments, the LNP (or liposome, nanoliposome, lipoplexe) comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In embodiments, the neutral lipid is 1,2-distearoyl-sn-glycero-3-phosphocholine (DSPC). Suitably, the molar ratio of the cationic lipid to DSPC may be in the range from about 2:1 to about 8:1.

In embodiments, the steroid is cholesterol. Suitably, the molar ratio of the cationic lipid to cholesterol may be in the range from about 2:1 to about 1:1. In some embodiments, the cholesterol may be PEGylated.

The sterol may be cholesterol. The sterol is typically present at around 25 to 55 molar %.

The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

The cationic lipid of an LNP may be cationisable, i.e. it becomes protonated as the pH is lowered below the pK of the ionizable group of the lipid but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease. A range of suitable ionizable cationic lipids are known in the art, which are typically present at around 20 to 60 molar %.

Such lipids (for liposomes, lipid nanoparticles (LNP), lipoplexes, and/or nanoliposomes) include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)-N,N-dimethyl-2,2-di((9Z,12Z)-octadeca-9,12-dienyl)tetrahydro-3aH-cyclopenta[d] [1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010053572 (and particularly, CI 2-200 described at paragraph [00225]) and WO2012170930, both of which are incorporated herein by reference, HGT4003, HGT5000, HGTS001, HGT5001, HGT5002 (see US20150140070A1).

In embodiments, the cationic lipid of the liposomes, lipid nanoparticles (LNP), lipoplexes, and/or nanoliposomes may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In embodiments, the cationic lipid of the liposomes, lipid nanoparticles (LNP), lipoplexes, and/or nanoliposomes may an aminoalcohol lipidoid.

Aminoalcohol lipidoids may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017075531A1, hereby incorporated by reference.

In another embodiment, suitable lipids can also be the compounds as disclosed in WO2015074085A1 (i.e. ATX-001 to ATX-032 or the compounds as specified in claims 1-26), U.S. Appl. No. 61/905,724 and Ser. No. 15/614,499 or U.S. Pat. Nos. 9,593,077 and 9,567,296 hereby incorporated by reference in their entirety.

In other embodiments, suitable cationic lipids can also be the compounds as disclosed in WO2017117530A1 (i.e. lipids 13, 14, 15, 16, 17, 18, 19, 20, or the compounds as specified in the claims), hereby incorporated by reference in its entirety.

In some embodiments, ionizable or cationic lipids may also be selected from the lipids disclosed in WO2018078053A1 (i.e. lipids derived from formula I, II, and III of WO2018078053A1, or lipids as specified in Claims 1 to 12 of WO2018078053A1), the disclosure of WO2018078053A1 hereby incorporated by reference in its entirety. In that context, lipids disclosed in Table 7 of WO2018078053A1 (e.g. lipids derived from formula I-1 to I-41) and lipids disclosed in Table 8 of WO2018078053A1 (e.g. lipids derived from formula II-1 to 11-36) may be suitably used in the context of the invention. Accordingly, formula I-1 to formula 1-41 and formula II-1 to formula II-36 of WO2018078053A1, and the specific disclosure relating thereto, are herewith incorporated by reference.

In some embodiments, cationic lipids may be derived from formula III of published PCT patent application WO2018078053A1. Accordingly, formula III of WO2018078053A1, and the specific disclosure relating thereto, are herewith incorporated by reference.

In some embodiments, the mRNA is complexed with one or more lipids thereby forming LNPs (or liposomes, nanoliposomes, lipoplexes), wherein the cationic lipid of the LNP is selected from structures III-1 to III-36 of Table 9 of published PCT patent application WO2018078053A1. Accordingly, formula III-1 to III-36 of WO2018078053A1, and the specific disclosure relating thereto, are herewith incorporated by reference.

In some embodiments, the ionisable cationic lipid has the formula III:

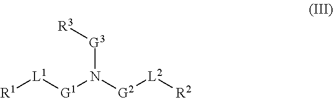

or a pharmaceutically acceptable salt, tautomer or stereoisomer thereof,
wherein:
L1 or L2 is each independently —O(C═O)— or —(C═O)O—;
G1 and G2 are each independently unsubstituted C1-C12 alkylene or C1-C12 alkenylene;
G3 is C1-C24 alkylene, C1-C24 alkenylene, C3-C8 cycloalkylene, or C3-C8 cycloalkenylene;
R1 and R2 are each independently C6-C24 alkyl or C6-C24 alkenyl;
R3 is H, OR5, CN, —C(═O)OR4, —OC(═O)R4 or —NR5C(═O)R4;
R4 is C1-C12 alkyl;
R5 is H or C1-C6 alkyl.

In some embodiments, the ionisable cationic lipid has the formula III:

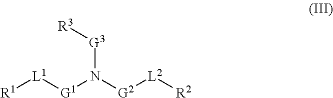

or a pharmaceutically acceptable salt, tautomer or stereoisomer thereof,
wherein:
L1 or L2 is each independently —O(C═O)— or —(C═O)O—;
G1 and G2 are each independently unsubstituted C1-C12 alkylene;
G3 is C1-C24 alkylene;
R1 and R2 are each independently C6-C24 alkyl;
R3 is OR5; and
R5 is H.

In some embodiments, the ionisable cationic lipid has the formula:

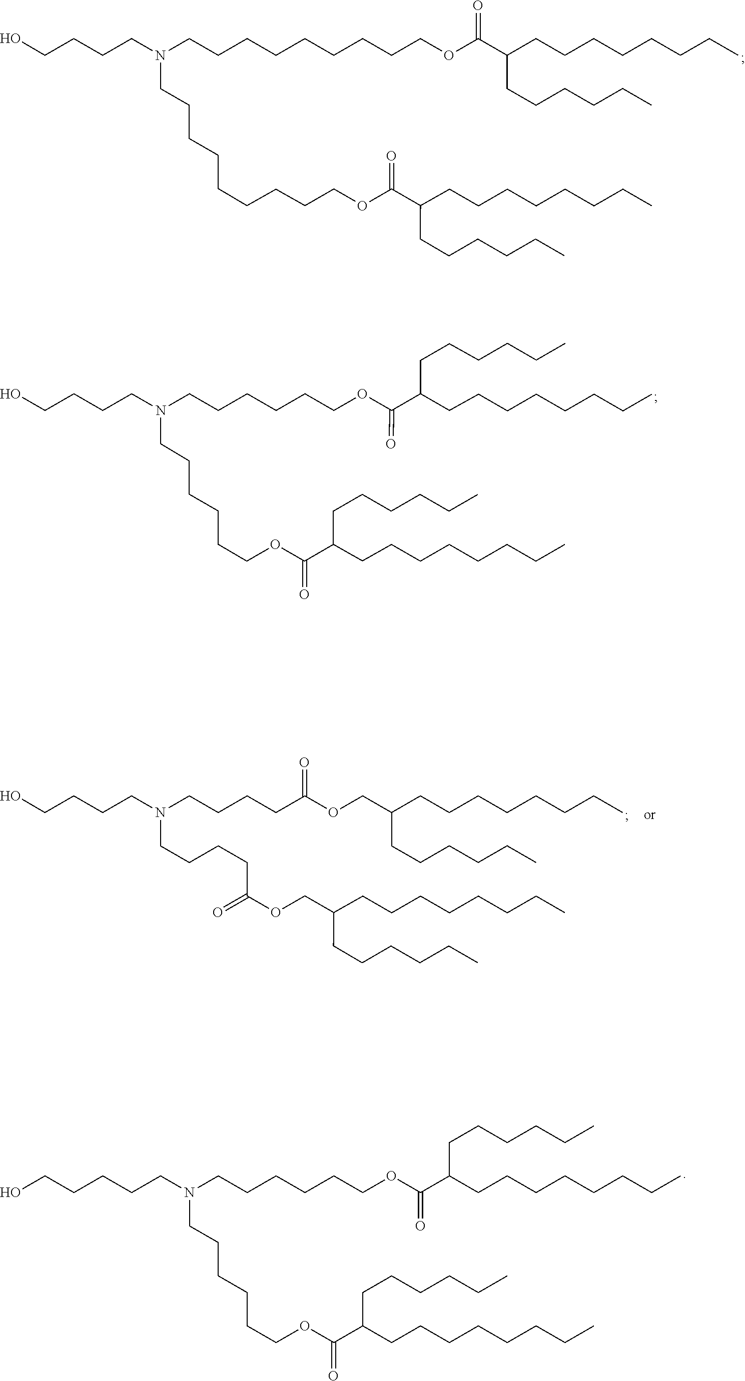 [SMILES: CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)COC(=O)CCCCN(CCCCO)CCCCC(=O)OCC(CCCCCC)CCCCCCCC ; CCCCCCCCC(CCCCCC)C(=O)OCCCCCCN(CCCCCO)CCCCCCOC(=O)C(CCCCCC)CCCCCCCC]

In some embodiments, the mRNA is complexed with one or more lipids thereby forming liposomes, lipid nanoparticles (LNP), lipoplexes, and/or nanoliposomes, suitably LNPs, wherein the liposomes, lipid nanoparticles (LNP), lipoplexes, and/or nanoliposomes, suitably the LNPs comprise a cationic lipid according to formula III-3:

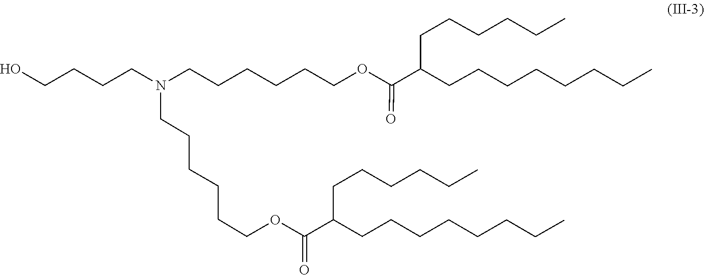 [SMILES: CCCCCCCCC(CCCCCC)C(=O)OCCCCCCN(CCCCO)CCCCCCOC(=O)C(CCCCCC)CCCCCCCC]

The lipid of formula III-3 as suitably used herein has the chemical term ((4-hydroxybutyl)azanediyl)bis(hexane-6,1-diyl)bis(2-hexyldecanoate), also referred to as ALC-0315 i.e. CAS Number 2036272-55-4.

In certain embodiments, the cationic lipid as defined herein, more suitably cationic lipid compound III-3 ((4-hydroxybutyl) azanediyl)bis(hexane-6,1-diyl)bis(2-hexyldecanoate)), is present in the LNP in an amount from about 30 mol % to about 80 mol %, suitably about 30 mol % to about 60 mol %, more suitably about 40 mol % to about 55 mol %, more suitably about 47.4 mol %, relative to the total lipid content of the LNP. If more than one cationic lipid is incorporated within the LNP, such percentages apply to the combined cationic lipids.

In some embodiments, the LNP comprises a cationic lipid having the following structure:

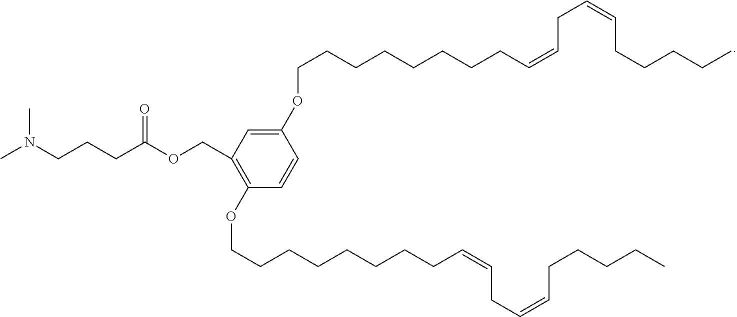 [SMILES: CCCCC/C=C\C/C=C\CCCCCCCCOc1ccc(OCCCCCCCC/C=C\C/C=C\CCCCC)c(COC(=O)CCCN(C)C)c1]

In embodiments, the cationic lipid is present in the LNP in an amount from about 30 mol % to about 70 mol %. In one embodiment, the cationic lipid is present in the LNP in an amount from about 40 mol % to about 60 mol %, such as about 40, 41, 42, 43, 44, 45, 46, 47, 48, 49, 50, 51, 52, 53, 54, 55, 56, 57, 58, 59 or 60 mol %, respectively. In embodiments, the cationic lipid is present in the LNP in an amount from about 47 mol % to about 48 mol %, such as about 47.0, 47.1, 47.2, 47.3, 47.4, 47.5, 47.6, 47.7, 47.8, 47.9, 50.0 mol %, respectively, wherein 47.4 mol % are particularly suitable.

In some embodiments, the cationic lipid is present in a ratio of from about 20 mol % to about 70 mol % or 75 mol % or from about 45 mol % to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid (e.g. coding RNA or DNA) is from about 3 to about 15, such as from about 5 to about 13 or from about 7 to about 11.

Other suitable (cationic or ionizable) lipids are disclosed in WO2009086558, WO2009127060, WO2010048536, WO2010054406, WO2010088537, WO2010129709, WO2011153493, WO 2013063468, US20110256175, US20120128760, US20120027803, U.S. Pat. No. 8,158,601, WO2016118724, WO2016118725, WO2017070613, WO2017070620, WO2017099823, WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, U.S. Pat. Nos. 7,893,302, 7,404,969, 8,283,333, 8,466,122 and 8,569,256 and US Patent Publication No. US20100036115, US20120202871, US20130064894, US20130129785, US20130150625, US20130178541, US20130225836, US20140039032 and WO2017112865. In that context, the disclosures of WO2009086558, WO2009127060, WO2010048536, WO2010054406, WO2010088537, WO2010129709, WO2011153493, WO 2013063468, US20110256175, US20120128760, US20120027803, U.S. Pat. No. 8,158,601, WO2016118724, WO2016118725, WO2017070613, WO2017070620, WO2017099823, WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, U.S. Pat. Nos. 7,893,302, 7,404,969, 8,283,333, 8,466,122 and 8,569,256 and US Patent Publication No. US20100036115, US20120202871, US20130064894, US20130129785, US20130150625, US20130178541, US20130225836 and US20140039032 and WO2017112865 specifically relating to (cationic) lipids suitable for LNPs (or liposomes, nanoliposomes, lipoplexes) are incorporated herewith by reference.

In other embodiments, the cationic or ionizable lipid is

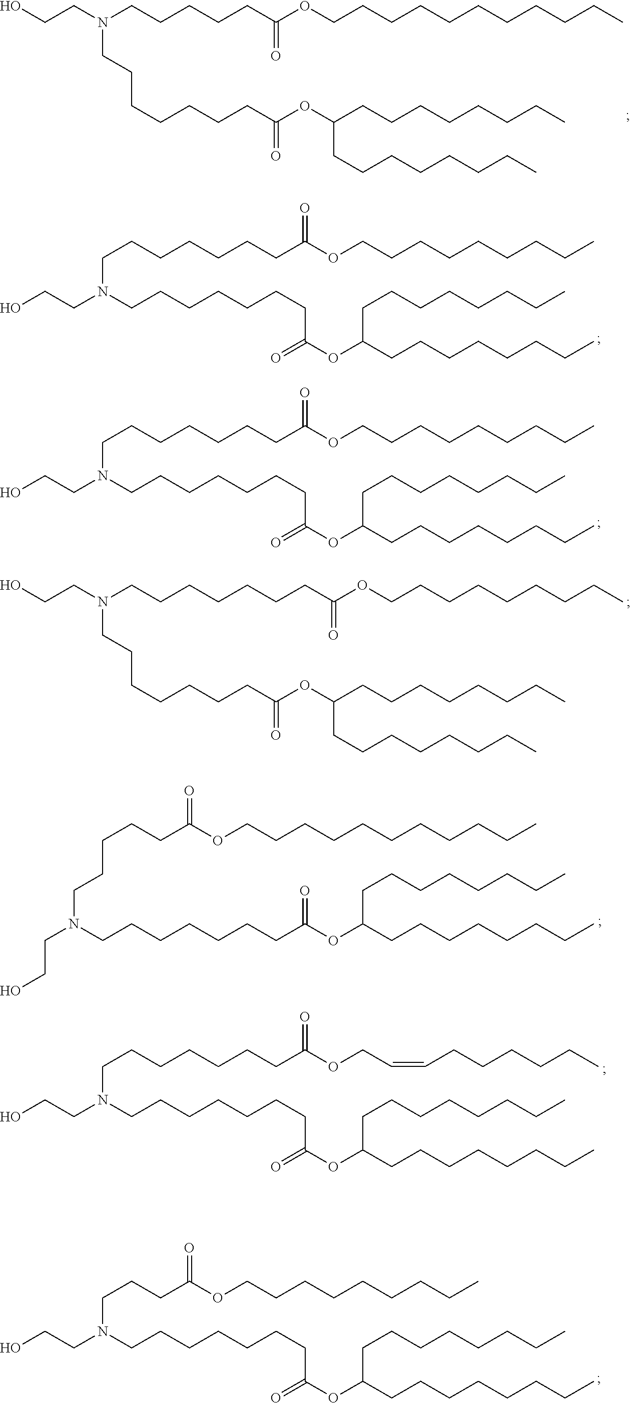

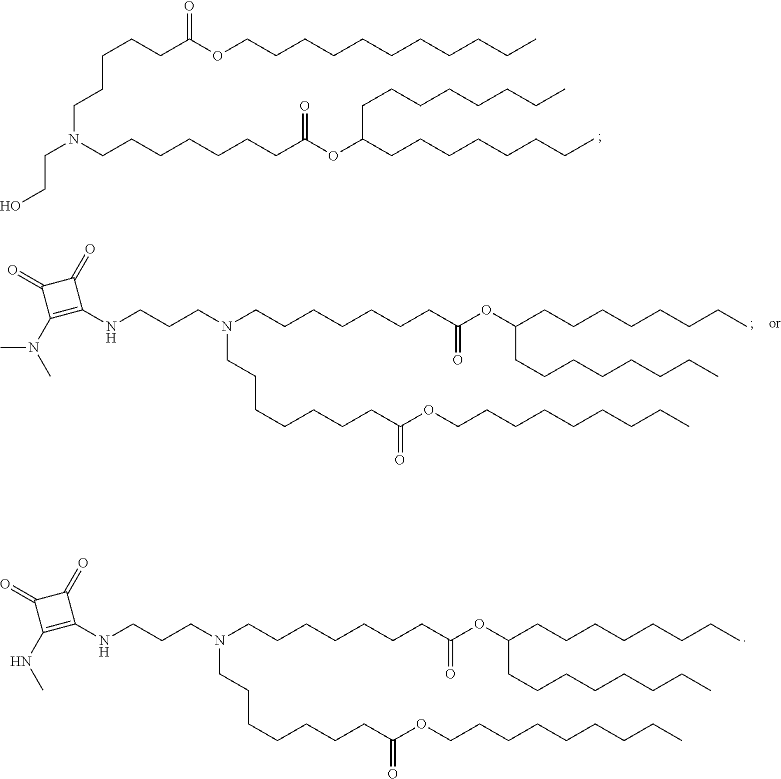

In embodiments, amino or cationic lipids as defined herein have at least one protonatable or deprotonatable group, such that the lipid is positively charged at a pH at or below physiological pH (e.g. pH 7.4), and neutral at a second pH, suitably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

LNPs (or liposomes, nanoliposomes, lipoplexes) can comprise two or more (different) cationic lipids as defined herein. Cationic lipids may be selected to contribute to different advantageous properties. For example, cationic lipids that differ in properties such as amine pKa, chemical stability, half-life in circulation, half-life in tissue, net accumulation in tissue, or toxicity can be used in the LNP (or liposomes, nanoliposomes, lipoplexes). In particular, the cationic lipids can be chosen so that the properties of the mixed-LNP are more desirable than the properties of a single-LNP of individual lipids.

The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20, or
(i) at an amount such as to achieve an N/P ratio in the range of about 1 to about 20, suitably about 2 to about 15, more suitably about 3 to about 10, even more suitably about 4 to about 9, most suitably about 6;
(ii) at an amount such as to achieve an N/P ratio in the range of about 5 to about 20, more suitably about 10 to about 18, even more suitably about 12 to about 16, most suitably about 14;
(iii) at an amount such as to achieve a lipid: mRNA weight ratio in the range of 20 to 60, suitably from about 3 to about 15, 5 to about 13, about 4 to about 8 or from about 7 to about 11; or
(iv) at an amount such as to achieve an N/P ratio in the range of about 6 for a lipid nanoparticle according to the invention, especially a lipid nanoparticle comprising the cationic lipid III-3.

In this context, the N/P ratio is defined as the mole ratio of the nitrogen atoms (“N”) of the basic nitrogen-containing groups of the lipid or lipidoid to the phosphate groups (“P”) of the nucleic acid which is used as cargo. The N/P ratio may be calculated on the basis that, for example, 1 μg RNA typically contains about 3 nmol phosphate residues, provided that the RNA exhibits a statistical distribution of bases. The “N”-value of the cationic lipid or lipidoid may be calculated on the basis of its molecular weight and the relative content of permanently cationic and—if present—cationisable groups. If more than one cationic lipid is present, the N-value should be calculated on the basis of all cationic lipids comprised in the lipid nanoparticles.

In one embodiment the lipid nanoparticles comprise about 40% cationic lipid LKY750, about 10% zwitterionic lipid DSPC, about 48% cholesterol, and about 2% PEGylated lipid DMG (w/w).

In some embodiments, lipid LNPs comprise:
(a) the mRNA of the invention, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the cationic lipids (as defined above), non-cationic lipids (as defined above), cholesterol (as defined above), and/or PEG-modified lipids (as defined above) may be combined at various relative molar ratios. For example, the ratio of cationic lipid to non-cationic lipid to cholesterol-based lipid to PEGylated lipid may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20:10, 40:30:25:5 or 40:32:20:8, 40:32:25:3 or 40:33:25:2, respectively.

In some embodiments, the LNPs (or liposomes, nanoliposomes, lipoplexes) comprise a lipid compound II (ALC-0315), the mRNA of the invention, a neutral lipid which is DSPC, a steroid which is cholesterol and a PEGylated lipid which is the compound of formula (I ALC-0159). In an embodiment, the LNP consists essentially of (i) at least one cationic lipid; (ii) a neutral lipid; (iii) a sterol, e.g., cholesterol; and (iv) a PEG-lipid, e.g. PEG-DMG or PEG-cDMA, in a molar ratio of about 20-60% cationic lipid:5-25% neutral lipid:25-55% sterol; 0.5-15% PEG-lipid.

In some embodiments, the mRNA is complexed with one or more lipids thereby forming lipid nanoparticles, wherein the LNP comprises
(i) at least one cationic lipid as defined herein, suitably lipid of formula III-3 (ALC-0315);
(ii) at least one neutral lipid as defined herein, suitably 1,2-distearoyl-sn-glycero-3-phosphocholine (DSPC);
(iii) at least one steroid or steroid analogue as defined herein, suitably cholesterol; and
(iv) at least one polymer conjugated lipid, suitably a PEG-lipid as defined herein, e.g. PEG-DMG or PEG-cDMA, suitably a PEGylated lipid that is or is derived from formula (I ALC-0159).

In some embodiments, the mRNA is complexed with one or more lipids thereby forming lipid nanoparticles (LNP), wherein the LNP comprises (i) to (iv) in a molar ratio of about 20-60% cationic lipid:5-25% neutral lipid:25-55% sterol; 0.5-15% polymer conjugated lipid, suitably PEG-lipid.

In some embodiments, the lipid nanoparticle (or liposome, nanoliposome, lipoplexe) comprises: a cationic lipid with formula (III-3) and/or PEG lipid with formula (IVa), optionally a neutral lipid, suitably 1,2-distearoyl-sn-glycero-3-phosphocholine (DSPC) and optionally a steroid, suitably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In an embodiment, the composition comprises the mRNA, lipid nanoparticles (LNPs), which have a molar ratio of approximately 50:10:38.5:1.5, suitably 47.5:10:40.8:1.7 or more suitably 47.4:10:40.9:1.7 (i.e. proportion (mol %) of cationic lipid (suitably lipid of formula III-3 (ALC-0315)), DSPC, cholesterol and polymer conjugated lipid, suitably PEG-lipid (suitably PEG-lipid of formula (I) with n=49, even more suitably PEG-lipid of formula (I) with n=45; ALC-0159); solubilized in ethanol).

The ratio of RNA to lipid can be varied (see for example WO2013/006825). In some embodiments, “N:P ratio” refers to the molar ratio of protonatable nitrogen atoms in the cationic lipids (typically solely in the lipid's headgroup) to phosphates in the RNA. The ratio of nucleotide (N) to phospholipid (P) can be in the range of, e.g., 1N:1P to 20N:1P, 1N:1P to 10N:1P, 2N:1P to 8N:1P, 2N:1P to 6N:1P or 3N:1P to 5N:1P. The ratio of nucleotide (N) to phospholipid (P) can be in the range of, e.g., 1N:1P, 2N:1P, 3N:1P, 4N:1P, 5N:1P, 6N:1P, 7N:1P, 8N:1P, 9N:1P, or 10N:1P. Alternatively or additionally, the ratio of nucleotide (N) to phospholipid (P) is 4N:1P.

WO2017/070620 provides general information on LNP compositions and is incorporated herein by reference. Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053, which are also incorporated herein by reference.

In various embodiments, LNPs that suitably encapsulates the mRNA of the invention have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average size as determined by dynamic light scattering as commonly known in the art.

LNPs are typically 50 to 200 nm in diameter (Z-average). Suitably the LNPs have a polydispersity of 0.4 or less, such as 0.3 or less. Typically, the PDI is determined by dynamic light scattering.

In some embodiments, the composition has a polydispersity index (PDI) value of less than about 0.4, suitably of less than about 0.3, more suitably of less than about 0.2, most suitably of less than about 0.1.

In one embodiment the carrier is a lipid nanoparticle (LNP).

CNE

The carrier may be a cationic nanoemulsion (CNE) delivery system. Such cationic oil-in-water emulsions can be used to deliver the mRNA to the interior of a cell. The emulsion particles comprise a hydrophobic oil core and a cationic lipid, the latter of which can interact with the mRNA, thereby anchoring it to the emulsion particle. In a CNE delivery system, the mRNA which encodes the antigen is complexed with a particle of a cationic oil-in-water emulsion. CNE carriers and methods for their preparation are described in WO2012/006380, WO2013/006837 and WO2013/006834 which are incorporated herein by reference.

Thus, the mRNA may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. Alternatively or additionally, the CNE comprises squalene and a cationic lipid, such as 1,2-dioleoyloxy-3-(trimethylammonio)propane (DOTAP) (see e.g. Brito, 2014). In an embodiment, the CNE is an oil-in-water emulsion of DOTAP and squalene stabilised with polysorbate 80 and/or sorbitan trioleate.

Desirably at least half of the RNA (and suitably at least 85%, such as all of it) is complexed with the cationic oil-in-water emulsion carrier.

CNE are typically 50 to 200 um in diameter (Z-average). Suitably the CNE have a polydispersity of 0.4 or less, such as 0.3 or less.

In one embodiment the carrier is a cationic nanoemulsion (CNE).

LION

A lipidoid-coated iron oxide nanoparticle (LION) is capable of delivering mRNA into cells and may be aided after administration to a subject by application of an external magnetic field. A LION is an iron oxide a particle with one or more coatings comprising lipids and/or lipidoids wherein mRNA encoding the antigen is incorporated into or associated with the lipid and/or lipidoid coating(s) through electrostatic interactions. The mRNA being embedded within the coating(s) may offer protection from enzymatic degradation. The lipids and/or lipidoids comprised within a LION may for example include those included in FIG. S1 of Jiang, 2013, especially lipidoids comprising alkyl tails of 12 to 14 carbons in length and in particular lipidoid C14-200 as disclosed in Jiang, 2013. A LION may typically comprise 200 to 5000, such as 500 to 2000, in particular about 1000 about 1000 lipid and/or lipidoid molecules. Typically the LIONs are 20 to 200 nm in diameter, especially 50 to 100 nm in diameter. The lipid/lipidoid to mRNA weight ratio may be about 1:1 to 10:1, especially about 5:1. Particularly suitable LIONs, and methods for preparation of LIONs are disclosed in Jiang, 2013.

In one embodiment the carrier is a lipidoid-coated iron oxide nanoparticle (LION).

Assays

The in vitro efficacy of vaccines which target the head region may be established by assays which investigate whether or not the vaccine prevents influenza virus from binding to target cells. An example of such an assay is the hemagglutination inhibition (HAI) assay, which is considered to be the gold standard in the field, and which provides a correlate of protection in vivo. However, vaccines which target the stem region, while being potentially protective, may not prevent influenza virus from binding to target cells. The above assays are therefore inappropriate for investigating the efficacy of a vaccine targeting the stem region.

Suitable assays for investigating the efficacy of a vaccine targeting the stem region which has been administered to mice are as follows. Implementations of these assays are used in the examples provided herein.

Anti-HA IgG Antibodies by ELISA

Quantification of mouse anti-HA IgG antibodies are performed by ELISA using HA antigen (full length or stem only) as coating. The plates are then incubated. Diluted sera are added to the coated plates and incubated. The plates are washed prior to the adding of diluted peroxidase conjugated goat anti-mouse IgG. The reaction is stopped with H₂SO₄ and optical densities are read. The titers are expressed as ELISA Units Titers.

Stem Specific T Cell Frequencies

Spleens are collected and cell suspensions are prepared. The splenic cell suspensions are filtered, harvested and centrifuged. Fresh splenocytes are then plated in the presence of an overlapping peptide pool covering the sequence of stem protein. Following stimulation, cells are washed and stained with anti-CD16/32, anti-CD4-V450 and anti-CD8-PerCp-Cy5.5 antibodies. Living/dead cell stain is added. Cells are permeabilized and stained with anti-IL2-FITC, anti-IFNγ-APC and anti-TNFα-PE antibodies. Stained cells are analyzed by flow cytometry.

Neutralization Antibody Titers

Mouse sera are diluted and incubated in the presence of reporter influenza virus. After incubation, the serum-virus mix is added to cell culture. Influenza-positive cells are analysed and quantified by flow cytometry. Titers are expressed as 50% neutralization titers (IC50), corresponding to reduction titers calculated by regression analysis of the inverse dilution of serum that provides 50% cell infected reduction compared to control wells (virus only, no serum).

More specific implementations of the above assays are detailed in the examples. These more specific assays may also be used for investigating the efficacy of a vaccine targeting the stem region.

Subjects

The present invention is generally intended for mammalian subjects, in particular human subjects. The subject may be a wild or domesticated animal. Mammalian subjects include for example cats, dogs, pigs, sheep, horses or cattle. In one embodiment of the invention, the subject is human.

The subject to be treated using the method of the invention may be of any age.

In one embodiment the subject is a human infant (up to 12 months of age). In one embodiment the subject is a human child (less than 18 years of age). In one embodiment the subject is an adult human (aged 18-59). In one embodiment the subject is an older human (aged 60 or greater).

Doses administered to younger children, such as less than 12 years of age, may be reduced relative to an equivalent adult dose, such as by 50%.

The methods of the invention are suitably intended for prophylaxis, i.e. for administration to a subject which is not infected with influenza virus.

Formulation and Administration

The carrier-formulated mRNA may be administered via various suitable routes, including parenteral, such as intramuscular or subcutaneous administration. Suitably the carrier-formulated mRNA is administered intramuscularly and/or intradermally.

In some embodiments, intramuscular administration of the carrier-formulated mRNA results in expression of the encoded antigen construct in a subject. Administration of the carrier-formulated mRNA results in translation of the mRNA and to a production of the encoded stem HA antigen in a subject.

The carrier-formulated mRNA may be provided in liquid or dry (e.g. lyophilised) form. The preferred form will depend on factors such as the precise nature of the carrier-formulated mRNA, e.g. if the carrier-formulated mRNA is amenable to drying, or other components which may be present.

The carrier-formulated mRNA is typically provided in liquid form.

In embodiments, the mRNA formulation described herein may be lyophilized in order to improve storage stability of the formulation and/or the mRNA. In embodiments, the mRNA formulation described herein may be spray dried in order to improve storage stability of the formulation and/or the mRNA. Lyoprotectants for lyophilization and or spray drying may be selected from trehalose, sucrose, mannose, dextran and inulin.

Suitably, the immunogenic composition, e.g. the composition comprising LNPs, is lyophilized (e.g. according to WO2016165831 or WO2011069586) to yield a temperature stable dried mRNA (powder) composition as defined herein. The composition, e.g. the composition comprising LNPs, may also be dried using spray-drying or spray-freeze drying (e.g. according to WO2016184575 or WO2016184576) to yield a temperature stable composition (powder) as defined herein.

Accordingly, in some embodiments, the pharmaceutical composition is a dried composition.

The term “dried composition” as used herein has to be understood as composition that has been lyophilized, or spray-dried, or spray-freeze dried as defined above to obtain a temperature stable dried composition (powder) e.g. comprising LNP complexed RNA (as defined above).

In embodiments, lyophilized or spray-dried composition has a water content of less than about 10%.

In some embodiments, lyophilized or spray-dried composition has a water content of between about 0.5% and 5%.

In some embodiments, the lyophilized or spray-dried composition is stable for at least 2 months after storage at about 5° C., suitably for at least 3 months, 4 months, 5 months, 6 months.

A composition comprising carrier-formulated mRNA intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, especially at least 5, in particular at least 5.5 such as at least 6. The pH of a formulation is generally 9 or less, especially 8.5 or less, in particular 8 or less, such as 7.5 or less. The pH of a formulation may be 4 to 9, especially 5 to 8.5, in particular 5.5 to 8, such as 6.5 to 7.4 (e.g. 6.5 to 7.1).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, especially 250 to 550 mOsm/kg, in particular 270 to 500 mOsm/kg, such as 270 to 400 mOsm/kg. Osmolality may be measured according to techniques known in the art, such as by the use of a commercially available osmometer, for example the Advanced® Model 2020 available from Advanced Instruments Inc. (USA).

Liquids used for reconstitution will be substantially aqueous, such as water for injection, phosphate buffered saline and the like. As mentioned above, the requirement for buffer and/or tonicity modifying agents will depend on the on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route. A typical human dose for administration through routes such as intramuscular, is in the region of 200 ul to 750 ml, such as 400 to 600 ul, in particular about 500 ul, such as 500 ul.

The carrier-formulated mRNA may be provided in various physical containers such as vials or pre-filled syringes.

In some embodiments the carrier-formulated mRNA is provided in the form of a single dose. In other embodiments the carrier-formulated mRNA is provided in multidose form such containing 2, 5 or 10 doses.

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul.

Stabilisers may be present. Stabilisers may be of particular relevance where multidose containers are provided as doses of the final formulation(s) may be administered to subjects over a period of time.

Formulations are preferably sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the carrier-formulated mRNA may therefore be part of a multi-dose administration regime. For example, the carrier-formulated mRNA may be provided as a priming dose in a multidose regime, especially a two- or three-dose regime, in particular a two-dose regime. The carrier-formulated mRNA may be provided as a boosting dose in a multidose regime, especially a two- or three-dose regime, such as a two-dose regime.

Priming and boosting doses may be homologous or heterologous. Consequently, the carrier-formulated mRNA may be provided as a priming dose and boosting dose(s) in a homologous multidose regime, especially a two- or three-dose regime, in particular a two-dose regime. Alternatively, the carrier-formulated mRNA may be provided as a priming dose or boosting dose in a heterologous multidose regime, especially a two- or three-dose regime, in particular a two-dose regime, and the boosting dose(s) may be different (e.g. carrier-formulated mRNA; or an alternative antigen presentation such as protein or virally vectored antigen—with or without adjuvant, such as squalene emulsion adjuvant).

The time between doses may be two weeks to six months, such as three weeks to three months. Periodic longer-term booster doses may be also be provided, such as every 2 to 10 years.

Accordingly, also provided is an immunogenic composition comprising the carrier-formulated mRNA according to the invention, wherein the composition optionally comprises at least one pharmaceutically acceptable carrier.

The term “pharmaceutically acceptable carrier” or “pharmaceutically acceptable excipient” as used herein suitably includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or suitably a buffer, more suitably an aqueous buffer, may be used, containing a sodium salt, suitably at least 50 mM of a sodium salt, a calcium salt, suitably at least 0.01 mM of a calcium salt, and optionally a potassium salt, suitably at least 3 mM of a potassium salt. According to some embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂, CaI₂, CaBr₂, CaCO₃, CaSO₄, Ca(OH)₂.

Furthermore, organic anions of the aforementioned cations may be in the buffer. Accordingly, in embodiments, the pharmaceutical composition may comprise pharmaceutically acceptable carriers or excipients using one or more pharmaceutically acceptable carriers or excipients to e.g. increase stability, increase cell transfection, permit the sustained or delayed, increase the translation of encoded antigenic peptides or proteins in vivo, and/or alter the release profile of encoded antigenic peptides or proteins protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one nucleic acid of component A and/or component B and, optionally, a plurality of nucleic acids of the composition, in such a manner that no interaction occurs, which would substantially reduce the biological activity or the pharmaceutical effectiveness of the composition under typical use conditions (e.g., intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The at least one pharmaceutically acceptable carrier or excipient of the immunogenic composition may be selected to be suitable for intramuscular or intradermal delivery/administration of the immunogenic composition. The immunogenic composition is suitably a composition suitable for intramuscular administration to a subject.

Subjects to which administration of the immunogenic compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In various embodiments, the immunogenic composition does not exceed a certain proportion of free mRNA.

In this context, the term “free mRNA” or “non-complexed mRNA” or “non-encapsulated mRNA” comprise the RNA molecules that are not encapsulated in the lipid-based carriers as defined herein.

During formulation of the composition (e.g. during encapsulation of the RNA into the lipid-based carriers), free RNA may represent a contamination or an impurity.

In embodiments, the immunogenic composition comprises free mRNA ranging from about 30% to about 0%. In embodiments, the composition comprises about 20% free mRNA (and about 80% encapsulated mRNA), about 15% free mRNA (and about 85% encapsulated mRNA), about 10% free mRNA (and about 90% encapsulated mRNA), or about 5% free mRNA (and about 95% encapsulated mRNA). In some embodiments, the composition comprises less than about 20% free mRNA, suitably less than about 15% free mRNA, more suitably less than about 10% free mRNA, most suitably less than about 5% free mRNA.

The term “encapsulated mRNA” comprises the mRNA molecules that are encapsulated in the lipid-based carriers as defined herein. The proportion of encapsulated mRNA in the context of the invention is typically determined using a RiboGreen assay.

In some embodiments, the composition is a multivalent composition comprising a plurality or at least one further mRNA in addition to the mRNA of the invention.

In some embodiments, the multivalent composition comprises two or more mRNA of the invention, suitably each encoding a different influenza HA stem polypeptide. In some embodiments, the multivalent composition comprises two, three or four mRNA. In some embodiments, the multivalent composition comprises two, three or four mRNA each encoding a different influenza HA stem polypeptide.

In some embodiments, the two or more mRNA encode influenza HA stem polypeptides derived from influenza A, such as influenza A Group 1 and/or influenza A Group 2.

In some embodiments, at least one of the two or more mRNA encodes an influenza HA stem polypeptide derived from influenza A Group 1, suitably influenza A subtype H1, H2, H5, H6, H8, H9, H11, H12, H13, H16, H17 and/or H18, more suitably H1; and at least one of the two or more mRNA encodes an influenza HA stem polypeptide derived from influenza A Group 2, suitably influenza A subtype H3, H4, H7, H10, H14 and/or H15, more suitably H3, H7 and/or H10, still more suitably H3.

In some embodiments, at least one of the two or more mRNA encodes an influenza HA stem polypeptide derived from influenza A subtype H1; and at least one of the two or more mRNA encodes an influenza HA stem polypeptide derived from influenza A subtype H3, H7 and/or H10.

In some embodiments, at least one of the two or more mRNA encodes an influenza HA stem polypeptide derived from influenza A subtype H1; and at least one of the two or more mRNA encodes an influenza HA stem polypeptide derived from influenza A subtype H3.

In some embodiments, at least one of the two or more mRNA encodes an influenza HA stem polypeptide derived from influenza A subtype H1; and at least one of the two or more mRNA encodes an influenza HA stem polypeptide derived from influenza A subtype H10.

In some embodiments, the influenza HA stem polypeptide derived from influenza A Group 1 comprises or consists of an amino acid sequence having at least 90%, 95%, 98% or 99% identity to the amino acid sequence set forth in any one of SEQ ID NO:1 or SEQ ID NO: 2 suitably SEQ ID NO: 2. In some embodiments, the influenza HA stem polypeptide derived from influenza A Group 1 comprises or consists of an amino acid sequence having at least 90%, 95%, 98% or 99% identity to the amino acid sequence set forth in SEQ ID NO: 2.

In some embodiments, the influenza HA stem polypeptide derived from influenza A Group 1 comprises or consists of the amino acid sequence set forth in any one of SEQ ID NO:1 or SEQ ID NO: 2, suitably SEQ ID NO: 2. In some embodiments, the influenza HA stem polypeptide derived from influenza A Group 1 comprises or consists of the amino acid sequence set forth in SEQ ID NO: 2.

In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 6 or SEQ ID NO: 7.

In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 12.

In some embodiments, the mRNA comprises or consists of a nucleic acid sequence having at least 90%, 95%, 98%, 99% or 100% identity to the nucleic sequence set forth in any one of SEQ ID NO: 16 or SEQ ID NO: 17.

In some embodiments, the mRNA comprises or consists of a nucleic acid sequence having at least 90%, 95%, 98%, 99% or 100% identity to the nucleic sequence set forth in any one of SEQ ID NO: 22 or SEQ ID NO: 23.

In some embodiments the influenza HA stem polypeptide derived from influenza A Group 2 comprises or consists of an amino acid sequence having at least 90%, 95%, 98% or 99% identity to the amino acid sequence set forth in any one of SEQ ID NO: 3, SEQ ID NO: 4, or SEQ ID NO: 10. In some embodiments, the influenza HA stem polypeptide derived from influenza A Group 2 comprises or consists of the amino acid sequence set forth in any one of SEQ ID NO: 3, SEQ ID NO: 4 or SEQ ID NO: 10.

In some embodiments, the influenza stem polypeptide is comprised within a construct having a polypeptide sequence having 80% or greater, such as 90% or greater, such as 95% or greater, such as 98% or greater, such as 99% or greater sequence identity to any one of SEQ ID NO: 8, 9 or 11.

In some embodiments, the influenza stem polypeptide is comprised within a construct having a polypeptide sequence having 80% or greater, such as 90% or greater, such as 95% or greater, such as 98% or greater, such as 99% or greater sequence identity to any one of SEQ ID NO: 13, 14 or 15.

In some embodiments, the mRNA comprises or consists of a nucleic acid sequence having at least 90%, 95%, 98%, 99% or 100% identity to the nucleic sequence set forth in any one of SEQ ID NO: 18 to 21.

In some embodiments, the mRNA comprises or consists of a nucleic acid sequence having at least 90%, 95%, 98%, 99% or 100% identity to the nucleic sequence set forth in any one of SEQ ID NO: 24 to 29.

In some embodiments, at least one of the two or more mRNA are non-replicating. In some embodiments, each of the two or more mRNA are non-replicating.

Also provided is a vaccine comprising the mRNA and/or the immunogenic composition.

In some embodiments, the vaccine is a multivalent vaccine comprising a plurality or at least more than one of the RNA of the invention, or a plurality or at least more than one of the composition.

Further provided is a kit or kit of parts comprising the mRNA, and/or the composition, and/or the vaccine, optionally comprising a liquid vehicle for solubilising, and, optionally, technical instructions providing information on administration and dosage of the components.

The technical instructions of the kit may contain information about administration and dosage and patient groups. Such kits, suitably kits of parts, may be applied e.g. for any of the applications or uses mentioned herein, suitably for the use of the immunogenic composition or the vaccine, for the treatment or prophylaxis of an infection or diseases caused by an Influenza virus, suitably Influenza A virus.

In some embodiments, the immunogenic composition or the vaccine is provided in a separate part of the kit, wherein the immunogenic composition or the vaccine is suitably lyophilised or spray-dried or spray-freeze dried.

The kit may further contain as a part a vehicle (e.g. buffer solution) for solubilising the dried or lyophilized nucleic composition or the vaccine.

In some embodiments, the kit or kit of parts as defined herein comprises a multi-dose container for administration of the composition/the vaccine and/or an administration device (e.g. an injector for intramuscular and/or intradermal injection).

Any of the above kits may be used in a treatment or prophylaxis as defined herein.

Also provided is the carrier-formulated mRNA, the immunogenic composition, the vaccine or the kit or kit of parts for use as a medicament.

It is furthermore provided several applications and uses of the carrier-formulated mRNA, the immunogenic composition, the vaccine, or the kit.

Therefore, further provided is the carrier-formulated mRNA, the immunogenic composition, the vaccine or the kit or kit of parts for use in the treatment or prophylaxis of an infection with an influenza virus, suitably an influenza A virus.

In some embodiments, the amount of carrier-formulated mRNA for each carrier-formulated mRNA is essentially equal in mass. In other embodiments, the amount of nucleic acid for each nucleic acid species is selected to be equimolar.

In some embodiments, a single dose of the carrier-formulated mRNA is 0.001 to 1000 μg, 0.01 to 1000 μg, especially 1 to 500 μg, in particular 10 to 250 μg total mRNA. In further embodiments, a single dose of the carrier-formulated mRNA comprises a mixture of 3, 4, 5, 6, 7, 8, 9 or 10 different mRNA and is 0.01 to 100 μg, especially 0.25 to 250 μg, in particular 0.5 to 25 μg of each mRNA.

In some embodiments, the carrier-formulated mRNA, the immunogenic composition, the vaccine, the kit or kit of parts for use is for intramuscular and/or intradermal administration suitably intramuscular administration.

In some embodiments, an immune response is elicited.

In some embodiments, an adaptative immune response is elicited.

In some embodiments, a protective adaptative immune response against an influenza virus is elicited.

In some embodiments, a protective adaptative immune response against an influenza A virus is elicited.

In some embodiments, a protective adaptative immune response against one or more influenza A virus subtype from Group 1 and/or Group 2 is elicited.

In some embodiments, the elicited immune response comprises neutralizing antibody titers against an influenza virus, suitably an influenza A virus, more suitably one or more influenza A virus subtype from Group 1 and/or Group 2.

In some embodiments, the elicited immune response comprises functional antibodies that can effectively neutralize the respective viruses.

In further embodiments, the elicited immune response comprises broad, functional cellular T-cell responses against the respective viruses. In particular, the elicited immune response comprises a CD4+ T cell immune response and/or a CD8+ T cell immune response.

In further embodiments, the elicited immune response comprises a well-balanced B cell and T cell response against the respective viruses.

In some embodiments, the elicited immune response comprises antigen-specific immune responses.

In some embodiments, the elicited immune response reduces partially or completely the severity of one or more symptoms and/or time over which one or more symptoms of influenza virus infection are experienced by the subject.

In some embodiments, the elicited immune response reduces the likelihood of developing an established influenza virus infection after challenge.

In some particular embodiments, the elicited immune response slows progression of influenza.

Also provided is a method of treating or preventing a disorder, wherein the method comprises applying or administering to a subject in need thereof the carrier-formulated mRNA, the composition, the vaccine or the kit or kit of parts.

Preventing (Inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection.

The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In some embodiments, the carrier-formulated mRNA, the composition, the vaccine or the kit or kit of parts is administered at a therapeutically effective amount.

In some embodiments, the disorder is an infection with an influenza virus, suitably an influenza A virus.

In some embodiments, the subject in need is a mammalian subject, suitably a human subject.

Also provided is a method of eliciting an immune response, wherein the method comprises applying or administering to a subject in need thereof the carrier-formulated mRNA, the composition, the vaccine or the kit or kit.

In some embodiments, an immune response is elicited.

In some embodiments, an adaptative immune response is elicited.

In some embodiments, a protective adaptative immune response against an influenza virus is elicited.

In some embodiments, a protective adaptative immune response against an influenza A virus is elicited.

In some embodiments, a protective adaptative immune response against one or more influenza A virus subtype from Group 1 and/or Group 2 is elicited.

In some embodiments, the elicited immune response comprises neutralizing antibody titers against an influenza virus, suitably an influenza A virus, more suitably one or more influenza A virus subtype from Group 1 and/or Group 2.

In some embodiments, the adaptive immune response comprises production of antibodies that bind to a HA protein that is not encoded by the carrier formulated mRNA.

In some embodiments, the elicited immune response comprises functional antibodies that can effectively neutralize the respective viruses.

In further embodiments, the elicited immune response comprises broad, functional cellular T-cell responses against the respective viruses.

In further embodiments, the elicited immune response comprises a well-balanced B cell and T cell response against the respective viruses.

In some embodiments, the immune response comprises a homologous, a heterologous and/or a heterosubtypic cross-reactive immunogenic responses against Influenza virus, suitably against Influenza A virus, more suitably against Influenza A virus subtypes of Group 1 and/or Group 2.

In some embodiments, the subject in need is a mammalian subject, suitably a human subject.

In embodiments, administration of the carrier-formulated mRNA, the composition, the vaccine or the kit or kit to a subject elicits neutralizing antibodies and does not elicit disease enhancing antibodies. In particular, administration of the carrier-formulated mRNA, the composition, the vaccine or the kit or kit to a subject does not elicit immunopathological effects, like e.g. enhanced disease and/or antibody dependent enhancement (ADE).

It has to be noted that specific features and embodiments that are described in the context of the carrier-formulated mRNA of the invention and/or the immunogenic composition of the invention are likewise applicable to the vaccine, the kit or kit of parts of the invention or further aspects including e.g. medical uses (first and second medical uses) and e.g. method of treatments.

### Further Definitions

For the sake of clarity and readability the following definitions are provided. Any technical feature mentioned for these definitions may be read on each and every embodiment of the invention. Additional definitions and explanations may be specifically provided in the context of these embodiments.

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus, a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given FIG., such as x±5% of the given FIG.

As used herein, the singular forms “a,” “an” and “the” include plural references unless the content clearly dictates otherwise.

Unless specifically stated, a process comprising a step of mixing two or more components does not require any specific order of mixing. Thus, components can be mixed in any order.

Where there are three components then two components can be combined with each other, and then the combination may be combined with the third component, etc.

Percentages in the context of numbers should be understood as relative to the total number of the respective items. In other cases, and unless the context dictates otherwise, percentages should be understood as percentages by weight (wt.-%).

The term “immunogenic fragment” or “immunogenic variant” has to be understood as any fragment/variant of the corresponding Influenza antigen that is capable of raising an immune response in a subject.

Adaptive immune response: The term “adaptive immune response” as used herein will be recognized and understood by the person of ordinary skill in the art, and is e.g. intended to refer to an antigen-specific response of the immune system (the adaptive immune system).

Antigen specificity allows for the generation of responses that are tailored to specific pathogens or pathogen-infected cells. The ability to mount these tailored responses is usually maintained in the body by “memory cells” (B-cells). In the context of the invention, the antigen is provided by the mRNA encoding at least one antigenic peptide or protein derived from Influenza virus.

Antigen: The term “antigen” as used herein will be recognized and understood by the person of ordinary skill in the art, and is e.g. intended to refer to a substance which may be recognized by the immune system, suitably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins comprising at least one epitope are understood as antigens in the context of the invention. In the context of the present invention, an antigen may be the product of translation of a provided mRNA as specified herein.

Antigenic peptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore, an antigenic/immunogenic peptide or protein comprises at least one epitope (as defined herein) or antigen (as defined herein) of the protein it is derived from (e.g. HA of influenza virus).

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”.

Cationisable: The term “cationisable” as used herein means that a compound, or group or atom, is positively charged at a lower pH and uncharged at a higher pH of its environment. Also, in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is suitable that it is positively charged at a pH value of about 1 to 9, suitably 4 to 9, 5 to 8 or even 6 to 8, more preferably of a pH value of or below 9, of or below 8, of or below 7, most suitably at physiological pH values, e.g. about 7.3 to 7.4, i.e. under physiological conditions, particularly under physiological salt conditions of the cell in vivo. In other embodiments, it is suitable that the cationisable compound or moiety is predominantly neutral at physiological pH values, e.g. about 7.0-7.4, but becomes positively charged at lower pH values. In some embodiments, the suitable range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be a DNA sequence, suitably an RNA sequence, consisting of a number of nucleotides that may be divided by three, which starts with a start codon and which suitably terminates with a stop codon.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if an RNA is “derived from” a DNA, in a first step DNA sequence is converted into the corresponding RNA sequence (in particular by replacing the T by U throughout the sequence). Thereafter, the sequence identity of the RNA sequences is determined. Suitably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Epitope: The term “epitope” (also called “antigen determinant” in the art) as used herein will be recognized and understood by the person of ordinary skill in the art, and is e.g. intended to refer to T cell epitopes and B cell epitopes. T cell epitopes or parts of the antigenic peptides or proteins and may comprise fragments suitably having a length of about 6 to about 20 or even more amino acids, e.g. fragments as processed and presented by MHC class I molecules, suitably having a length of about 8 to about 10 amino acids, e.g. 8, 9, or 10, (or even 11, or 12 amino acids), or fragments as processed and presented by MHC class II molecules, suitably having a length of about 13 to about 20 or even more amino acids. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, suitably having 5 to 15 amino acids, more suitably having 5 to 12 amino acids, even more suitably having 6 to 9 amino acids, which may be recognized by antibodies, i.e. in their native form. Such epitopes of proteins or peptides may furthermore be selected from any of the herein mentioned variants of such proteins or peptides. In this context epitopes can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Immunogen, immunogenic: The terms “immunogen” or “immunogenic” will be recognized and understood by the person of ordinary skill in the art, and are e.g. intended to refer to a compound that is able to stimulate/induce an immune response. In some embodiments, an immunogen is a peptide, polypeptide, or protein. An immunogen in the sense of the present invention is the product of translation of a provided nucleic acid, comprising at least one coding sequence encoding at least one antigenic peptide, protein derived from e.g. Influenza HA stem (suitably, Influenza A HA stem) as defined herein. Typically, an immunogen elicits an adaptive immune response.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be activated by ligands of pattern recognition receptor e.g. Toll-like receptors, NOD-like receptors, or RIG-1 like receptors etc.

Multivalent vaccine/composition: the multivalent vaccine or combination of the invention provides more than one valence (e.g. an antigen) derived from more than one virus (e.g. at least one Influenza virus as defined herein and at least one further Influenza virus as defined herein).

### EXAMPLES

Section 1—Examples with SAM Constructs

### Example 1—LNP Details and Mouse Immunisation

The LNPs used in the examples herein were ‘RV39’ lipid nanoparticles (composed of 40% cationic lipid LKY750, 10% zwitterionic lipid DSPC, 48% cholesterol, and 2% PEGylated lipid DMG (w/w)). These LNPs were used to produce LNP-formulated recombinant self-amplifying mRNA (SAM) replicons, encoding the HA stem from various influenza strains stabilized on a bacteria ferritin from H. pylori (monodisplay). The HA stem-ferritin fusion gene was generated by fusing the ectodomain of HA to H. pylori ferritin with a Ser-Gly-Gly linker.

Study A

The immunogenicity of a stem HA H1 candidate vaccine was evaluated in CB6F1 mice. Ten female CB6F1 mice were immunized at days 0 and 28 with:
(a) SAM-stem H1 A/Michigan/45/2015 (a SAM encoding the stem HA H1 A/Michigan/45/2015 polypeptide and H. pylori ferritin (SEQ ID NO: 7)) comprised within LNPs,
(b) QIV (commercially available quadrivalent influenza vaccine comprising inactivated split influenza virions of the strains A/Brisbane/02/2018 H1N1pdm09, A/Kansas/14/2017 H3N2, B/Colorado/06/2017 (B/Victoria) and B/Phuket/3073/2013 (B/Yamagata)) without adjuvant,
(c) QIV formulated with AS03, or
(d) NaCl solution.

Serum samples were collected and analysed as described in examples 3 to 7 below using the assay protocols described in example 2.

Non-inferiority can be concluded if the lower limit (LL) of the 90% CI for the ratio of the GMTs (GMR) between the compared groups is >0.5. Biological/clinical significance (non-inferiority margin) can be concluded if the GMR+90% CI is >0.5. Statistical superiority can be concluded if the GMR+90% CI is >2.

Study B

A further subsequent study, analogous to Study A above, was conducted to investigate the impact of administering different doses of SAM encoded stem HA and SAM encoded stem HA polypeptide derived from different strains of influenza. Female CB6F1 mice were immunized with:
(a) SAM-stem H1 A/Michigan/45/2015 (a SAM encoding the stem HA H1 A/Michigan/45/2015 polypeptide and H. pylori ferritin (SEQ ID NO: 7)) comprised within LNPs,
(b) SAM-stem H1 A/New Caledonia/20/99 (a SAM encoding the stem HA H1 A/New Caledonia/20/99 polypeptide and H. pylori ferritin (SEQ ID NO: 6)) comprised within LNPs,
(c) SAM-stem H10 A/Jiangxi-Donghu/346/2013 (a SAM encoding the stem HA H10 A/Jiangxi-Donghu/346/2013 polypeptide and H. pylori ferritin (SEQ ID NO: 9)) comprised within LNPs,
(d) QIV without adjuvant,
(e) QIV formulated with 25 uL AS03, or
(f) NaCl solution.

Fourteen mice were included per groups (a)-(e) and four mice were included in group (f). Serum samples were collected and analysed as described in examples 3 to 7 below using the assay protocols described in example 2.

Non-inferiority can be concluded if the lower limit (LL) of the 90% CI for the ratio of the GMTs (GMR) between the compared groups is >0.5. Biological/clinical significance (non-inferiority margin) can be concluded if the GMR+90% CI is >0.5. Statistical superiority can be concluded if the GMR+90% CI is >2.

### Example 2—Assay Protocols

Anti-HA IgG Antibodies by ELISA

Quantification of mouse anti-HA IgG antibodies was performed by ELISA using HA antigen (full length or stem only) as coating diluted at a concentration of 4 μg/ml in PBS (50 μl/well). The plates were then incubated for 1 hour at 37° C. in saturation buffer. Diluted sera were added to the coated plates (50 μl/well) and incubated for 90 minutes at 37° C. The plates were washed prior to the adding of diluted peroxydase conjugated goat anti-mouse IgG. The reaction was stopped with H2SO4 2N and optical densities were read at 490-620 nm. The titers were expressed as ELISA Units Titers (EU/ml).

Stem Specific T Cell Frequencies

Spleens were collected and placed in complemented RPMI Cell suspensions were prepared from each spleen using a tissue grinder. The splenic cell suspensions were filtered, harvested, centrifuged and resuspended in Complete Medium. Fresh splenocytes were then plated in 96-well plates in presence of overlapping peptide pool covering the sequence of H1 Mich 15 stem. Following stimulation, cells were stained and analyzed using a 5-colour ICS assay. Cells were washed and stained with anti-CD16/32, anti-CD4-V450 and anti-CD8-PerCp-Cy5.5 antibodies. Live/dead-PO was added for 30 min at 4° C. Cells were permeabilized and stained with anti-IL2-FITC, anti-IFNγ-APC and anti-TNFα-PE antibodies. Stained cells were analyzed by flow cytometry using a LSRII and the FlowJo software.

Neutralization Antibody Titers

Quantification of mouse neutralizing antibody titers was assessed by microneutralization assay. Briefly, mouse sera were diluted and incubated in presence of reporter influenza virus.

After incubation, the serum-virus mix were added on cell culture. Influenza-positive cells were analysed and quantified by flow cytometry. Titers are expressed as 50% neutralization titers (IC50), corresponding to reduction titers calculated by regression analysis of the inverse dilution of serum that provided 50% cell infected reduction compared to control wells (virus only, no serum).

### Example 3—Anti-H1 Stem IgG Antibody Titers by ELISA at 14 Days Post Dose 2

IgG antibody titers directed towards H1-stem were measured by ELISA assay at 14 days post second immunization (day 42).

The results from Study A are shown in FIG. 1 High anti-H1 stem IgG antibodies were induced by SAM H1 stem, comparable to and even improved (1 μg) compared to titers induced by QIV/AS03 immunisation (SAM stem H1 1 μg/QIV: GMR 54.83 and LL 21.94; SAM stem H1 1 μg/QIV+AS03: GMR 8.60 and LL 3.08). ELISA titers are expressed as midpoint values (Geomean with 95% CI).

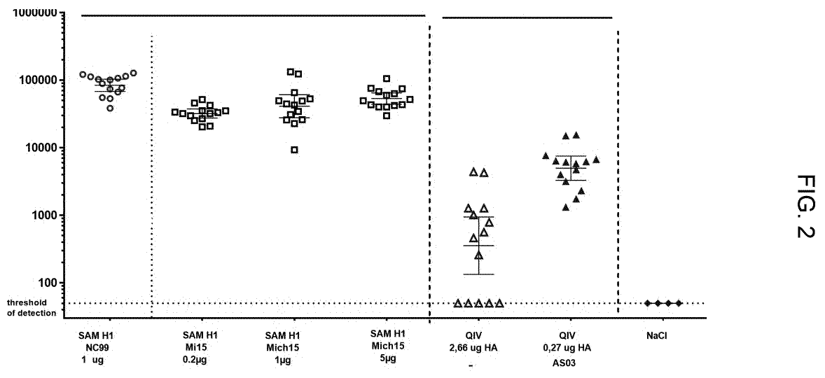

The results from Study B are shown in FIG. 2. High anti-H1 stem IgG antibodies were induced by SAM stem H1/NC/99, comparable to and even improved compared to titers induced by QIV/AS03 immunisation (SAM stem H1/NC/99/QIV: GMR 235.88 and LL 100.78; SAM stem H1/NC/99/QIV+AS03: GMR 16.86 and LL 12.34). High anti-H1 stem IgG antibodies were induced by SAM stem H1/Mich/15, comparable to and even improved (0.2 μg, 1 μg and 5 μg) compared to titers induced by QIV/AS03 immunisation (SAM stem H1/Mich/15 0.2 μg/QIV: GMR 90.17 and LL 38.79; SAM stem H1/Mich/15 0.2 μg/QIV+AS03: GMR 6.45 and LL 4.83). ELISA titers are expressed as 50% endpoint titers (individual animals with GMT and IC95).

The dotted horizontal line on the FIG.s corresponds to the threshold of detection.

### Example 4—Anti-H1/NC/99 and Anti-H1/Mich/15 IgG Antibody Titers by ELISA at 14 Days Post Dose 2

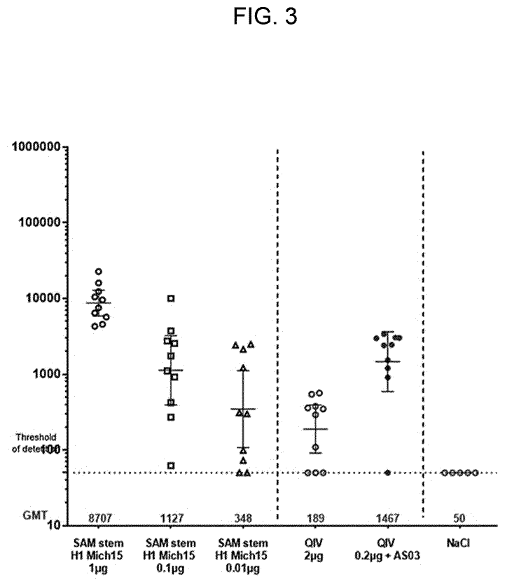

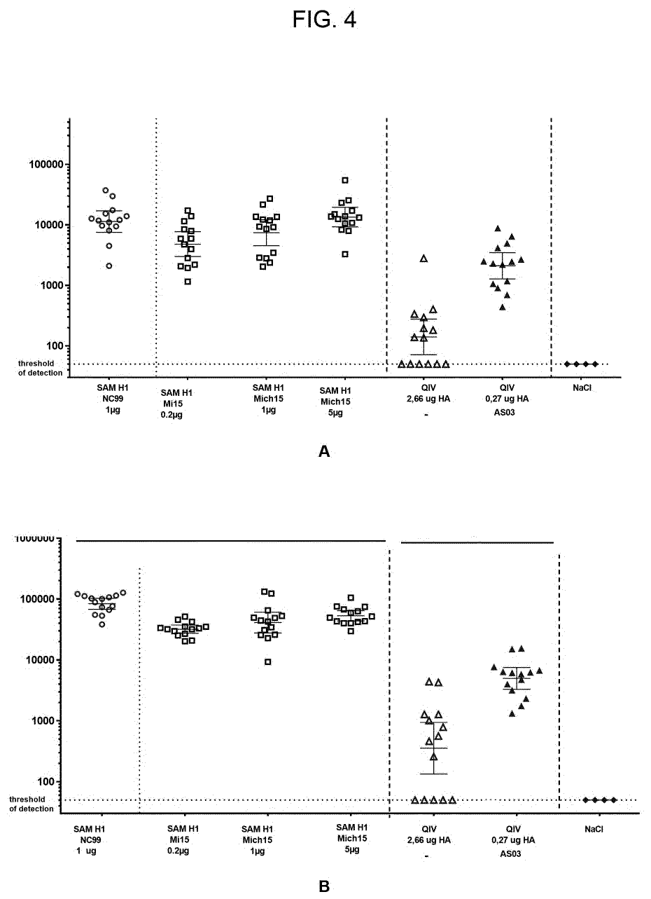

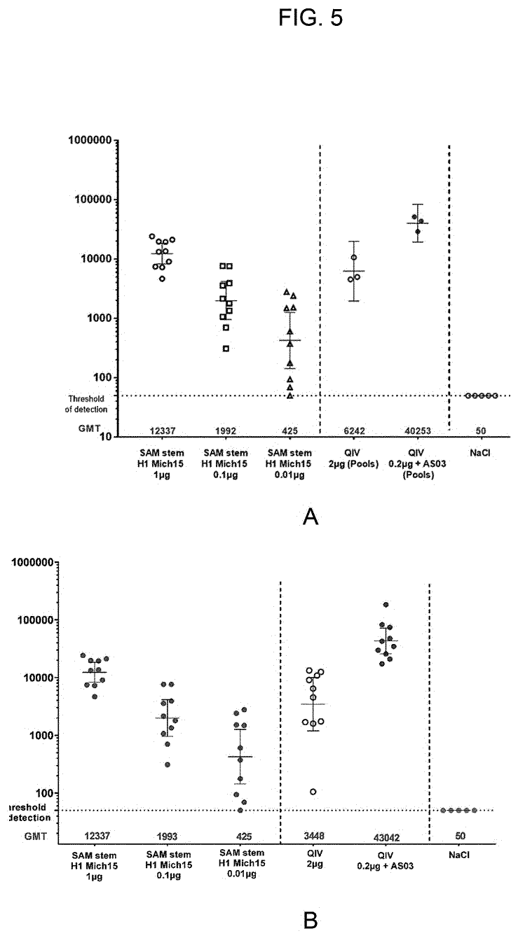

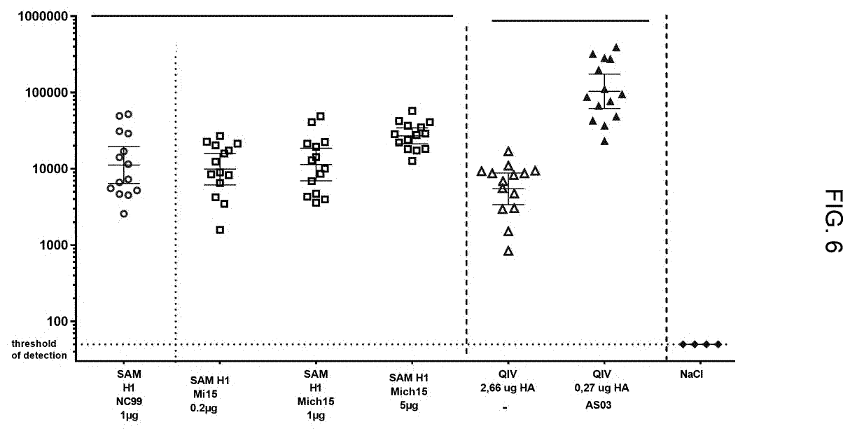

IgG antibody titers directed towards H1 were measured by ELISA assay using a full-length (trimeric protein with foldon and without transmembrane domain) A/H1N1/New Caledonia/20/1999 polypeptide (Study A, FIG. 3 and Study B, FIG. 4A) or a full-length A/H1N1/Michigan/2015 polypeptide (Study A, FIGS. 5A (QIV groups analyzed on pools) and 5B (QIV groups analyzed on individual sera) and Study B, FIG. 6) at 14 days post second immunization (day 42).

Study A has revealed that high anti-H1 NC99 IgG antibodies were induced by SAM H1 stem, improved (1 μg) compared to titers induced by QIV/AS03 immunisation (SAM stem H1 1 μg/QIV: GMR 46.10 and LL 21.00; SAM stem H1 ipg/QIV+AS03: GMR 5.94 and LL 2.30). High anti-H1 Mich15 IgG antibodies were induced by SAM H1 stem, improved (ipg) compared to titers induced by QIV immunisation (SAM stem H1/QIV: GMR 3.58 and LL 1.19).

Study B has revealed that high anti-H1/NC/99 IgG antibodies were induced by SAM stem H1/NC/99 and H1/Mich/15 (0.2 μg, 1 μg and 5 μg), and even improved compared to titers induced by QIV/AS03 immunisation (SAM H1/NC/99/QIV: GMR 80.64 and LL 42.74; SAM H1/NC/99/QIV+AS03: GMR 5.37 and LL 3.19; SAM H1/Mich/15 0.2 μg/QIV: GMR 34.20 and LL 17.60; SAM H1/Mich/15 0.2 μg/QIV+AS03: GMR 2.28 and LL 1.30). High anti-H1/Mich/15 IgG antibodies were induced by SAM stem H1/NC/99 and H1/Mich/15 (ipg and 5 μg), improved compared to titers induced by QIV immunisation (SAM H1/NC/99/QIV: GMR 2.05 and LL 1.12; SAM H1/Mich/15 ipg/QIV: GMR 2.08 and LL 1.19).

In Study B only, the experiment was repeated using a stem-only A/H1N1/New Caledonia/20/1999 polypeptide as coating antigen. The results are shown in FIG. 4B.

For FIG. 3 and FIGS. 5A et 5B, ELISA titers are expressed as midpoint values (Geomean with 95% CI). For FIGS. 4A and 4B and FIG. 6, ELISA titers are expressed as 50% endpoint titers (individual animals with GMT and IC95).

The dotted horizontal line on the FIG.s corresponds to the threshold of detection.

### Example 5—Anti-Group A1 (H2, H9, H18) IgG Antibody Titers by ELISA at 14 Days Post Dose 2

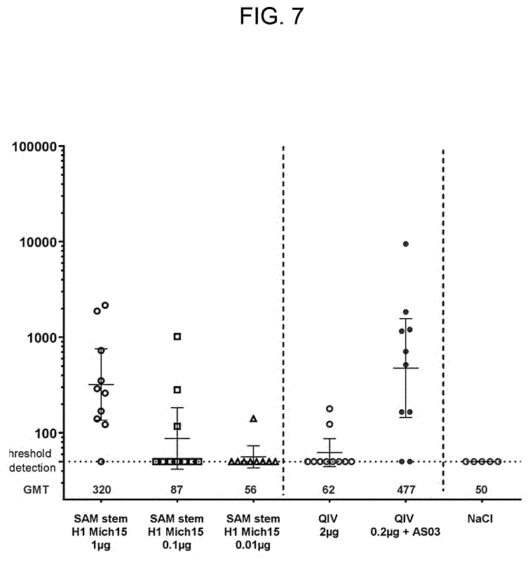

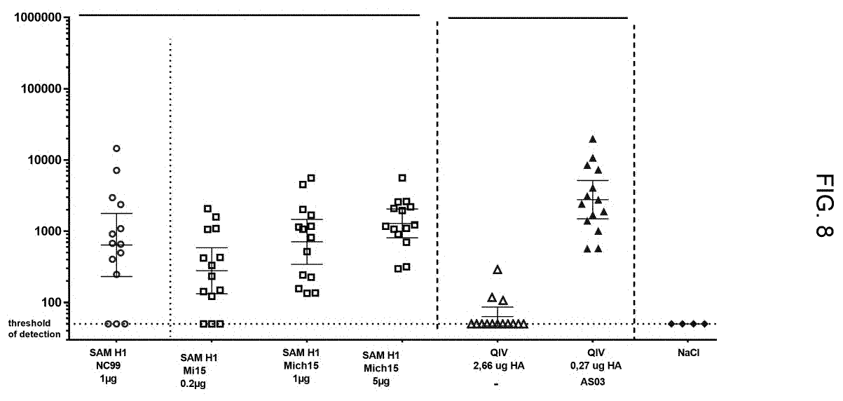

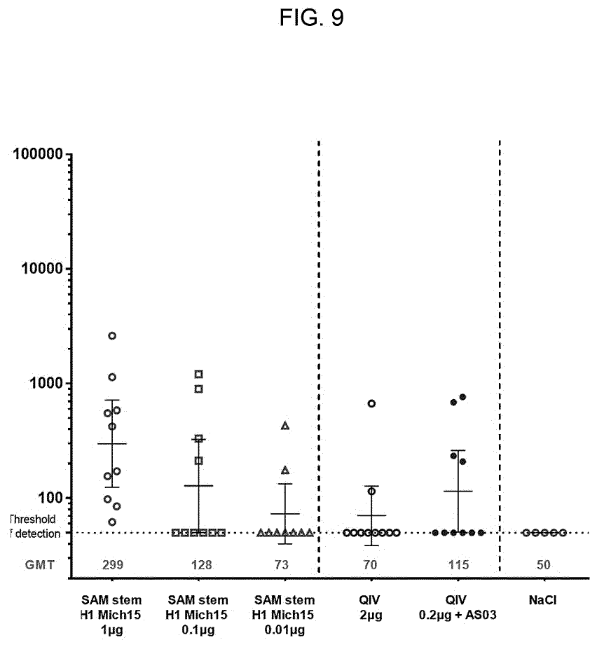

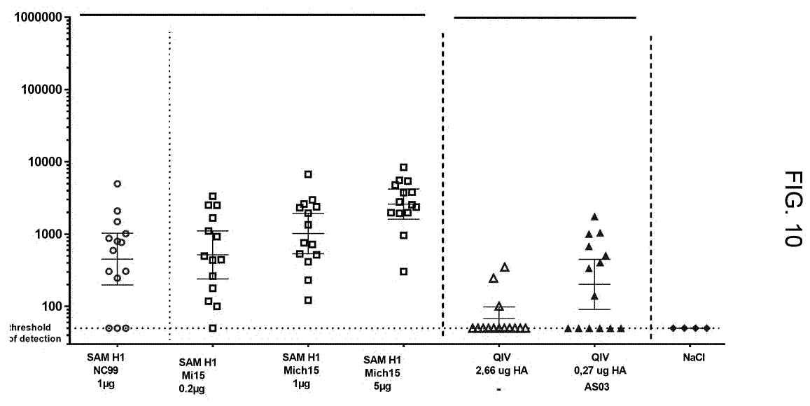

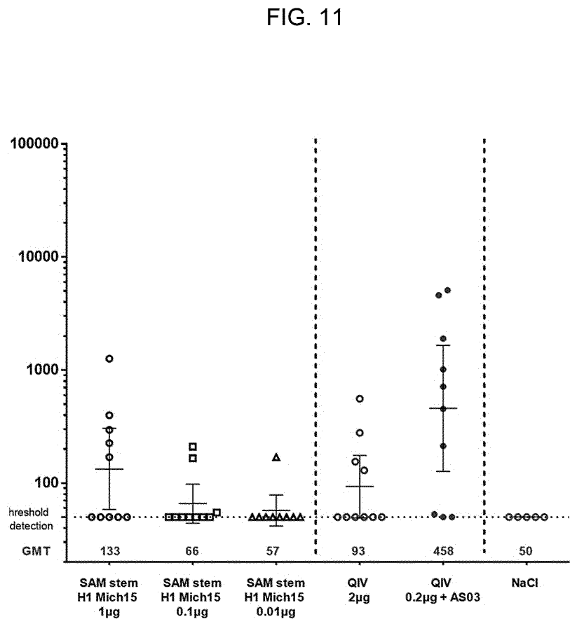

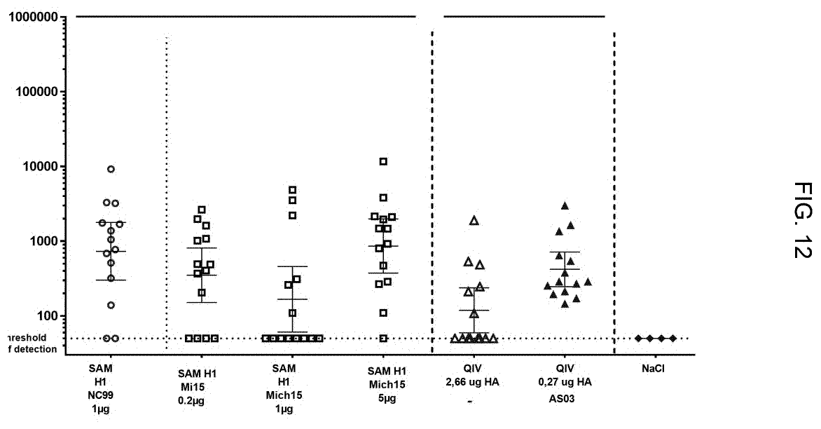

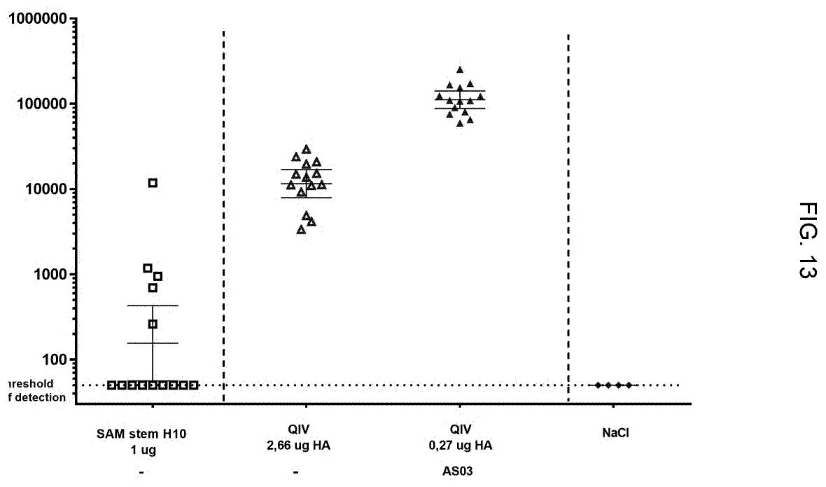

IgG antibody titers directed towards group A1 HA were measured by ELISA assay using a full-length H2 (Study A, FIG. 7 and Study B, FIG. 8), a full-length H9 (Study A, FIG. 9 and Study B, FIG. 10) or a full-length H18 (Study A, FIG. 11 and Study B, FIG. 12) at 14 days post second immunization (day 42).

Study A has revealed that anti-H2, anti-H9 and anti-H18 IgG antibodies are induced by SAM-stem H1.

Study B has revealed that anti-H2 IgG antibodies are induced by SAM-stem H1/NC/99 and H1/Mich/15 (0.2 μg, 1 μg and 5 μg), and even improved compared to titers induced by QIV immunisation (SAM H1/NC/99/QIV: GMR 10.07 and LL 4.09; SAM H1/Mich/15 0.2 μg/QIV: GMR 4.38 and LL 2.25).

Study B has further revealed that anti-H9 IgG antibodies are induced by SAM-stem H1/NC/99 and H1/Mich/15 (0.2 μg, 1 μg and 5 μg), and even improved compared to titers induced by QIV/AS03 immunisation (SAM H1/NC/99/QIV: GMR 6.66 and LL 3.11; SAM H1/Mich/15 0.2 μg/QIV: GMR 7.63 and LL 3.74; SAM H1/NC/99/QIV+AS03: GMR 2.23 and LL 0.89; SAM H1/Mich/15 0.2 μg/QIV+AS03: GMR 2.55 and LL 1.06).

Study B has further revealed that anti-H18 IgG antibodies are induced by SAM-stem H1/NC/99 and H1/Mich/15 (0.2 μg, 1 μg and 5 μg), and even improved compared to titers induced by QIV immunisation (SAM H1/NC/99/QIV: GMR 6.17 and LL 2.62; SAM H1/Mich/15 0.2 μg/QIV: GMR 2.96 and LL 1.26).

For FIG. 7, FIG. 9 and FIG. 11, ELISA titers are expressed as midpoint values (Geomean with 95% CI). For FIG. 8, FIG. 10 and FIG. 12, ELISA titers are expressed as 50% endpoint titers (individual animals with GMT and IC95).

The dotted horizontal line on the FIG.s corresponds to the threshold of detection.

### Example 6—Anti-Group A2 (H3, H7, H10) IgG Antibody Titers by ELISA at 14 Days Post Dose 2

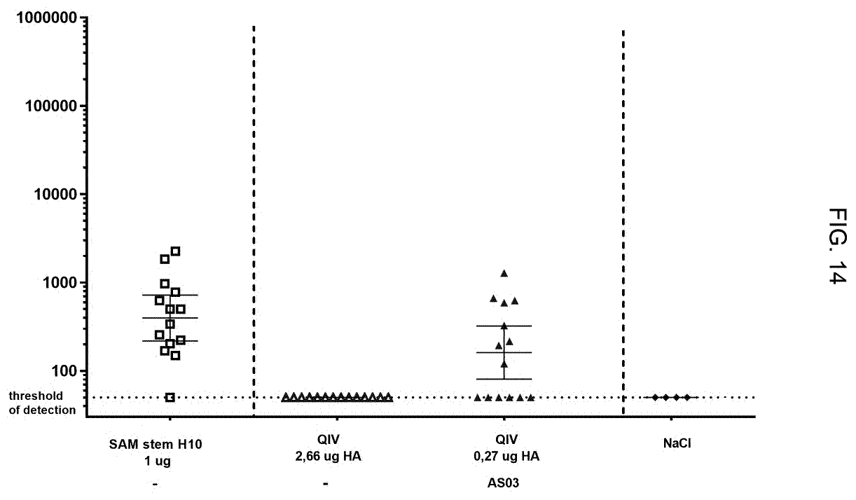

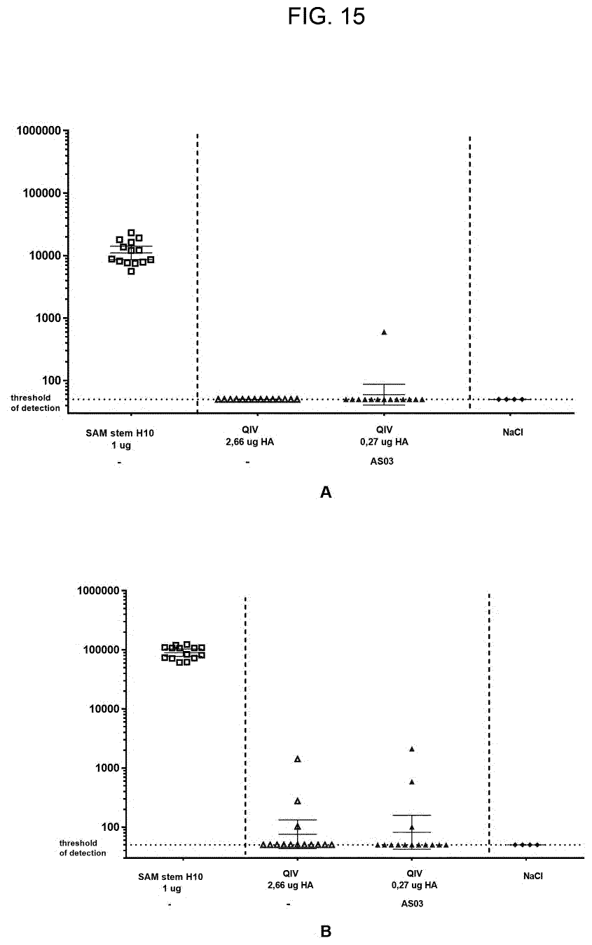

This experiment was carried out for Study B. IgG antibody titers directed towards group A2 HA were measured by ELISA assay using a full-length H3 protein (FIG. 13), a full-length H7 protein (FIG. 14) or a full length H10 protein (FIG. 15A) at 14 days post second immunization (day 42).

Study B has revealed that anti-H3 and anti-H10 IgG antibodies are induced by SAM-stem H10/Ji/13.

Study B has further revealed that anti-H7 IgG antibodies are induced by SAM-stem H10/Ji/13, and even improved compared to titers induced by QIV/AS03 immunisation (SAM H10/Ji/13/QIV+AS03: GMR 2.46 and LL 1.16).

The H10 ELISA experiment was repeated using a stem-only polypeptide as coating antigen. The results are shown in FIG. 15B.

ELISA titers are expressed as 50% endpoint titers (individual animals with GMT and IC95).

The dotted horizontal line on the FIG.s corresponds to the threshold of detection.

### Example 7—H1/Mich/15 Stem Specific CD4+ and CD8+ T Cells Frequencies at 14 Days Post Dose 2

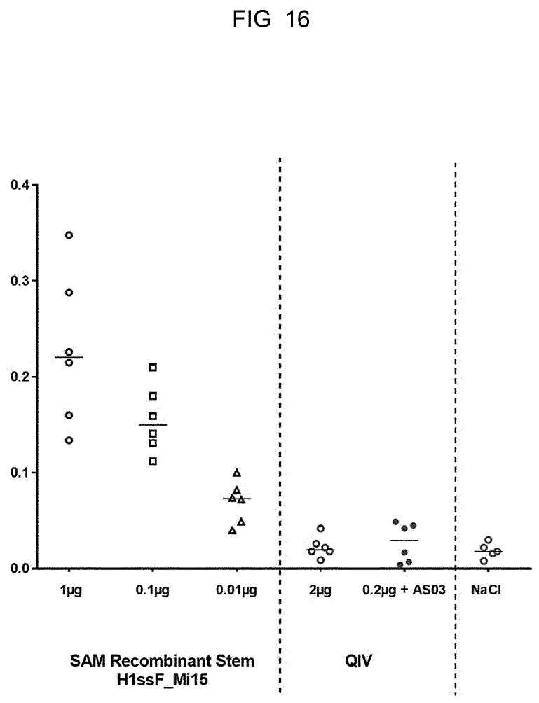

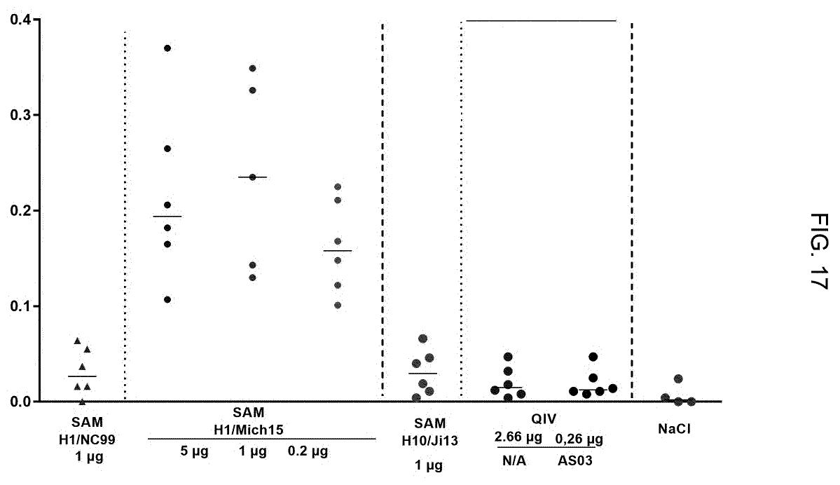

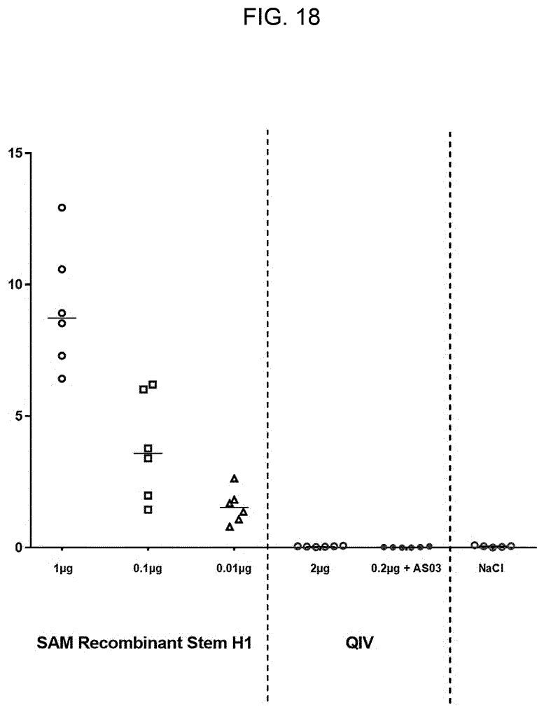

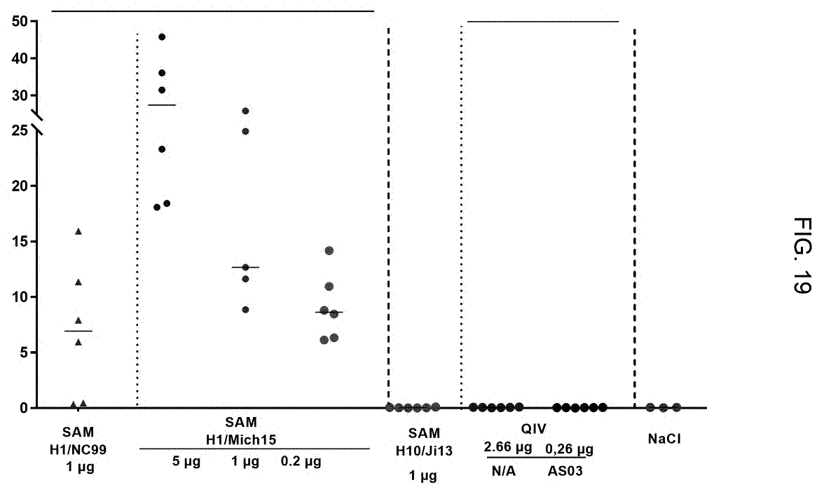

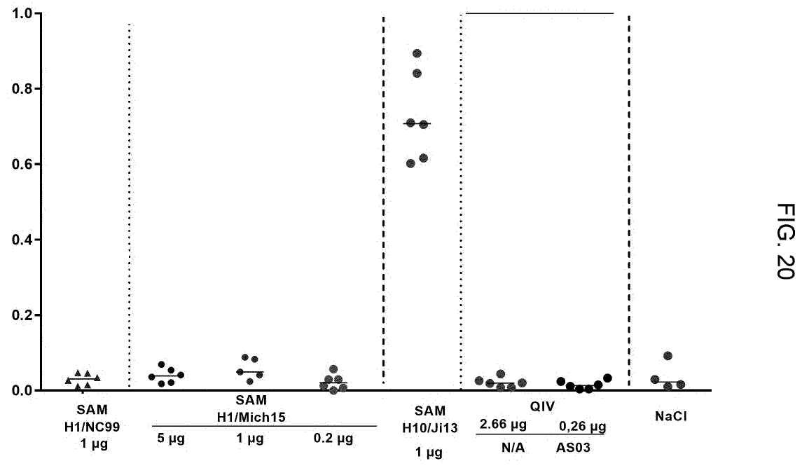

The T cell response induced by the stem H1 candidate vaccine was evaluated. The percentage of H1 stem-specific CD4+ T cells (Study A, FIG. 16 and Study B, FIG. 17) and CD8+ T cells (Study A, FIG. 18 and Study B, FIG. 19) were measured 14 days after the second immunization. Intracellular staining was performed on splenocytes after a 6 hours re-stimulation with peptide pools covering the sequence of H1 stem (A/Michigan/45/2015).

For all the studies, higher frequencies of H1/mich/15 stem specific CD4+ T cell were observed with the SAM-stem H1 antigen as compared to QIV with or without AS03 (e.g. Study B—SAM H1/Mich/15 0.2 μg/QIV: GMR 10.58 and LL 6.52; SAM H1/Mich/15 0.2 μg/QIV+AS03: GMR 9.85 and LL 6.39).

For FIG. 16, the results are expressed as percentage of H1 A/Michigan/45/2015 stem-specific CD4+ T cells expressing IFNγ and/or IL2 and/or TNFα and/or IL13 and/or IL17 (individual animals with medians).

For FIG. 17, the results are expressed as percentage of stem H1 FLU pool of peptides-specific CD4+ T cells expressing IFNγ and/or IL2 and/or TNFα (individual animals with medians).

For all the studies, higher frequencies of H1/Mich/15 stem specific CD8+ T cell were observed with the SAM-stem H1 antigen as compared to QIV with or without AS03 (e.g. Study B—SAM H1/NC/99/QIV: GMR 59.82 and LL 19.56; SAM H1/NC/99/QIV+AS03: GMR 106.61 and LL 32.30; H1/Mich/15 0.2 μg/QIV: GMR 158.44 and LL 110.40; SAM H1/Mich/15 0.2 μg/QIV+AS03: GMR 282.38 and LL 134.11).

For FIG. 18, the results are expressed as percentage of H1 A/Michigan/45/2015 stem-specific CD8+ T cells expressing IFNγ and/or IL2 and/or TNFα and/or IL13 and/or IL17 (individual animals with medians).

For FIG. 19, the results are expressed as percentage of stem H1 FLU pool of peptides-specific CD8+ T cells expressing IFNγ and/or IL2 and/or TNFα (individual animals with medians).

The dotted horizontal line on the FIG.s corresponds to the threshold of detection.

### Example 8—H10/Jiangxi-Donghu Stem Specific CD4+ and CD8+ T Cells Frequencies at 14 Days Post Dose 2

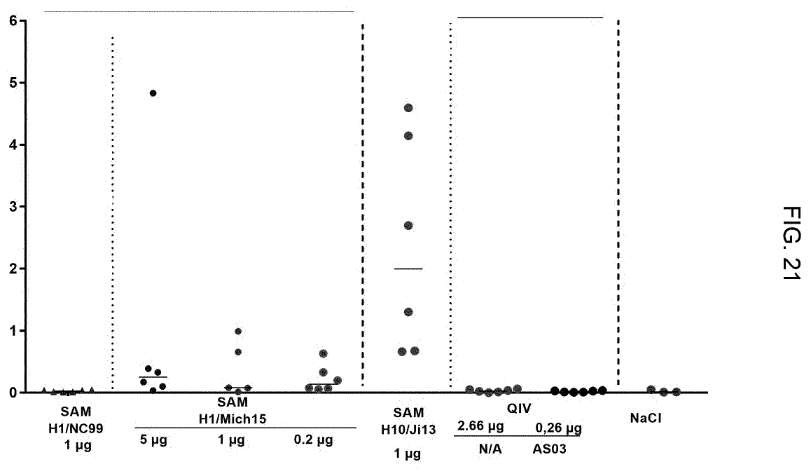

This experiment was carried out for Study B only. The percentage of H10 stem-specific CD4+ T cells (FIG. 20) and CD8+ T cells (FIG. 21) were measured 14 days after the second immunization. Intracellular staining was performed on splenocytes after a 6 hours re-stimulation with peptide pools covering the sequence of H10 stem (H10/Jiangxi-Donghu).

Higher frequencies of H1/NC/99, H1/mich/15 (1 μg) and H10/Ji/13 stem specific CD4+ T cell were observed with the SAM-stem H10 antigen as compared to QIV with AS03 (SAM H1/NC/99/QIV+AS03: GMR 2.32 and LL 1.19; SAM H1/Mich/15 1 μg/QIV+AS03: GMR 4.65 and LL 2.23; SAM H10/Ji/13/QIV+AS03: GMR 63.69 and LL 35.53).

Higher frequencies of H1/Mich/15 and H10/Ji/13 stem specific CD8+ T cell were observed with the SAM-stem H10 antigen as compared to QIV with or without AS03 (SAM H10/Ji/13/QIV: GMR 112.08 and LL 23.70; SAM H10/Ji13/QIV+AS03: GMR 101.58 and LL 47.44; H1/Mich/15 0.2 μg/QIV: GMR 9.63 and LL 1.91; SAM H1/Mich/15 0.2 μg/QIV+AS03: GMR 8.72 and LL 3.23).

For FIG. 20, the results are expressed as percentage of stem H10 FLU pool of peptides-specific CD4+ T cells expressing IFNγ and/or IL2 and/or TNFα (individual animals with medians).

For FIG. 21, the results are expressed as percentage of stem H10 FLU pool of peptides-specific CD8+ T cells expressing IFNγ and/or IL2 and/or TNFα (individual animals with medians).

The dotted horizontal line on the FIG.s corresponds to the threshold of detection.

### Example 9—Group A1 H1/Mich/15, H1/NC/99 and H5/Vn/04 Microneutralization Titers at 14 Days Post Dose 2

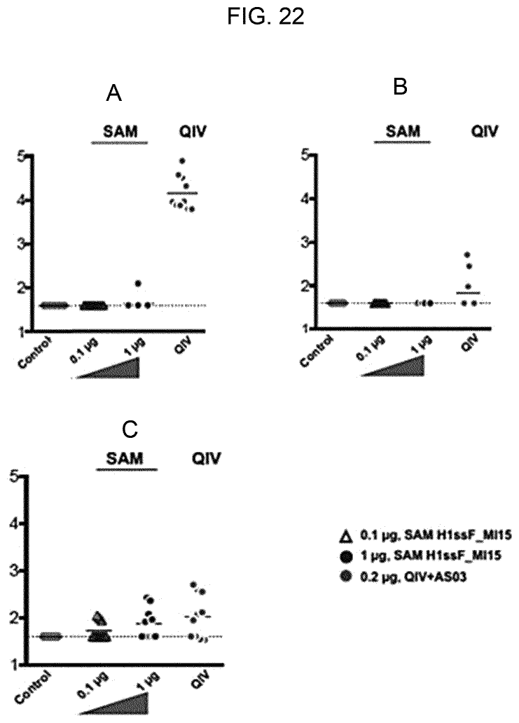

Microneutralization titers towards group A1 influenza virus were measured by microneutralisation assay using H1/Mich/15 (panel A), H1/NC/99 (panel B) or H5/Vn/04 (panel C) reporter viruses (FIG. 22). The results are expressed as IC50 (log₁₀ dilution).

The dotted horizontal line on the FIG.s corresponds to the threshold of detection.

Section 2—Examples with Non-Replicating mRNA Constructs

For all the Examples under Section 2, the H1 constructs were based on the A/Michigan/45/2015 (H1N1) strain (e.g. SEQ ID NO: 7 and/or SEQ ID NO: 12) and the H3 constructs were based on the A/Finland/486/2004 (H3N2) strain (e.g. SEQ ID NO: 8 and/or SEQ ID NO: 13).

The HA-stem constructs were provided either with a ferritin from H. pylori (F or Fe) or with a transmembrane domain (TM).

The HA-stem constructs were further provided with natural leader/signal peptide (ferritin or TM constructs) or HLA-DRa leader (TM constructs).

Section 2.1—Examples with Unmodified Nucleosides

### Example 10—In Vitro Translation of HA-Stem Constructs

In vitro translation of mRNA constructs was performed using the Promega Rabbit Reticulocyte Lysate System and canine pancreatic microsomal membranes. RNA is linearized for 3 min at 65° C. and immediately put on ice. Then, 0.2 μg mRNA (or water=mock) are incubated in a 25 μl reaction with rabbit reticulocyte lysate, amino acids, RNase inhibitor, and biotinylated Lysyl-tRNA according to manufacturer's instructions. One reaction contains canine microsomal membranes in addition to the translation components. Reactions are incubated at 30° C. for 90 min. Protein sample buffer is added to the reactions. Samples are separated on 4-20% gradient gels by SDS-PAGE and transferred to PVDF-FL membrane by western blot. Membranes are blocked with Intercept Blocking Buffer in TBS. For antibody dilution Blocking buffer is diluted in TBS and 0.2% Tween-20+0.01% SDS are added. In vitro translation products are visualized using IRDye 800CW-conjugated streptavidin antibody (1:2000 in 0.5× Intercept/TBS/0.2% Tween-20/0.01% SDS). Membranes are incubated with antibody solution for 1 h at room temperature and washed 3× with TBS/0.2% Tween-20/0.01%. Bands are detected with Odyssey CLx image system.

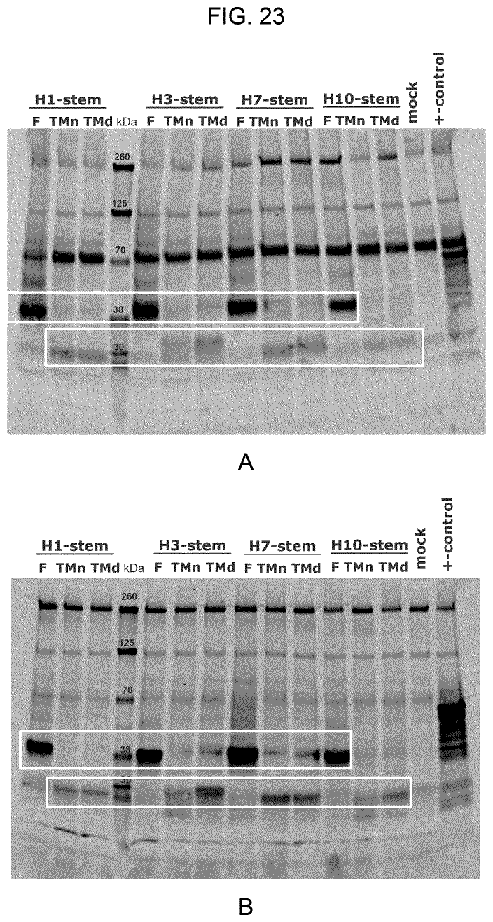

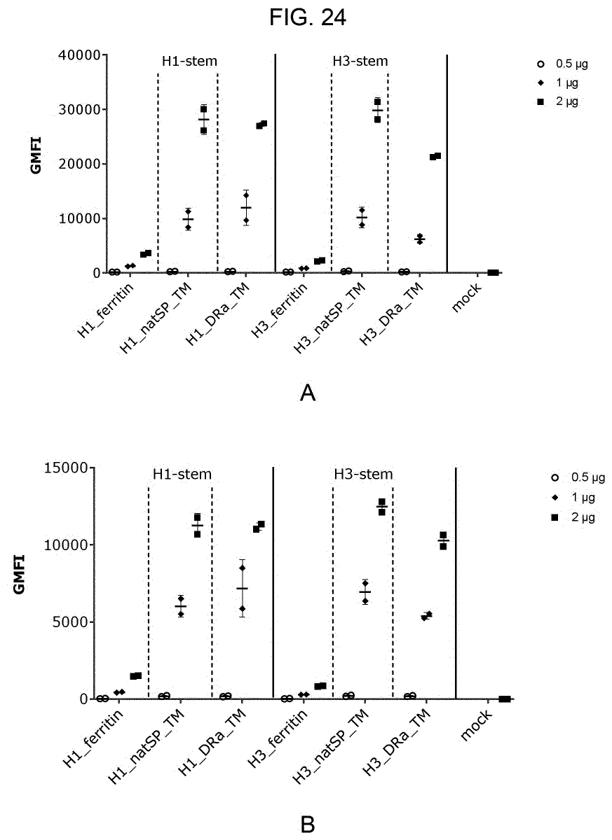

Results are shown in FIG. 23A (in vitro translation with membranes—ivt w/membranes) and 23B (in vitro translation without membranes—ivt w/o membranes). Ferritin constructs are translated more efficiently than transmembrane (TM) constructs. No difference was observed between the TM designs. All proteins are glycosylated when membranes are present (higher weight on FIG. 23A).

### Example 11—In Vitro HA-Stem Trimer Expression in Tissues Culture

HeLa cells were seeded at a density of 4×10⁵ cells/well in 2 ml medium in a 6-well plate. The next day, mRNAs were transfected with Lipofectamine 2000 in duplicate according to manufacturer's instructions. For each well, 0.5, 1, or 2 μg of mRNA were mixed with 0.75, 1.5, or 3 μl Lipofectamine 2000 (1:1.5 ratio) in a total of 500 μl Opti-MEM media and added to the cells. After 18-24 h cells were harvested and used for staining.

Transfected HeLa cells were washed with PBS and incubated with detach buffer (40 mM Tris-HCl pH7.5, 150 mM NaCl, 1 mM EDTA) before transferring into Eppendorf tubes. Cells were washed with PBS, resuspended in 300 μl PBS, and divided into the 3 wells of a 96-V-bottom plate, so that cells of one well were used in three different stainings. All samples were incubated with 200 μl Aqua dye (1:1000 in PBS) for 30 min at 4° C. in the dark to differentiate live and dead cells, washed twice with 200 μl PBS/0.5% BSA and used for surface or intracellular staining.

For surface staining, cells were incubated with 100 μl of the respective monoclonal antibody (at a concentration of 10 μg/ml in PBS/0.5% BSA), or buffer only. Samples were incubated for 30 min at 4° C. in the dark, washed twice with 200 μl PBS/0.5% BSA, and incubated under the same conditions with 100 μl PE-labeled anti-human IgG antibody 1:200 in PBS/0.5% BSA. After antibody incubations, cells were washed twice with PBS/0.5% BSA, fixed with 1% Formaldehyde in PBS, and washed twice more. Cells were resuspended in PBEA (PBS+0.5% BSA+2 mM EDTA+0.01% NaN₃) and analyzed by flow cytometry using the ZE5 flow cytometer.

For intracellular staining, cells were fixed and permeabilized by treating with 200 μl Cytofix/Cytoperm for 30 min at 4° C. Cells were washed twice with Permwash and incubated with 100 μl of the respective monoclonal antibody (at a concentration of 10 μg/ml in Permwash), or Permwash only. Samples were incubated for 30 min at 4° C. in the dark, washed twice with 200 μl Permwash, and incubated under the same conditions with 100 μl PE-labeled anti-human IgG antibody 1:200 in Permwash. After antibody incubations, cells were washed twice with Permwash, resuspended in PBEA and analyzed by flow cytometry using the ZE5 flow cytometer.

Geometric mean flurescence intensity (GMFI) is plotted for each replicate. Lines indicate mean+/−standard deviation.

Results are shown on FIGS. 24A (cellular trimers) and 24B (surface trimers), obtained by using CT149 antibody. CT149 is a human-derived monoclonal antibody that recognizes the HA-stem of Group 1 and Group 2 HAs. It binds to two protomers of the same trimer and is therefore sensitive to the quaternary structure of the HA stem (Wu, 2015).

Ferritin design is much less expressed than TM versions. There is no difference between the signal peptides for either TM construct.

### Example 12—In Vitro H1-Stem Expression after Co-Transfection of H1- and H3-Stem mRNAs

HeLa cells were seeded at a density of 4×10⁵ cells/well in 2 ml medium in a 6-well plate. The next day, mRNAs were transfected with Lipofectamine 2000 in duplicate according to manufacturer's instructions. For each well a total of 2 μg of mRNA was mixed with 3 μl Lipofectamine 2000 (1:1.5 ratio) in a total of 500 μl Opti-MEM media and added to the cells. After 18-24 h cells were harvested and used for staining. Note, mRNAs were mixed equimolar as follows. MRNAs were weight adjusted to the heaviest mRNA (i.e., H3_ferritin). Lighter mRNAs were transfected with the same molarity and differences in total mRNA weight were compensated by addition of irrelevant mRNA (i.e., R1803 encoding for Rabies virus G protein). Each mRNA encoding an HA construct was weight adjusted to equal molarity of 1 μg of H3_ferritin mRNA and R1803 was added to a total amount of 2 μg mRNA. Cell staining and flow cytometry was performed as described for Example 11.

Geometric mean fluorescence intensity (GMFI) is plotted for each replicate. Lines indicate mean+/−standard deviation.

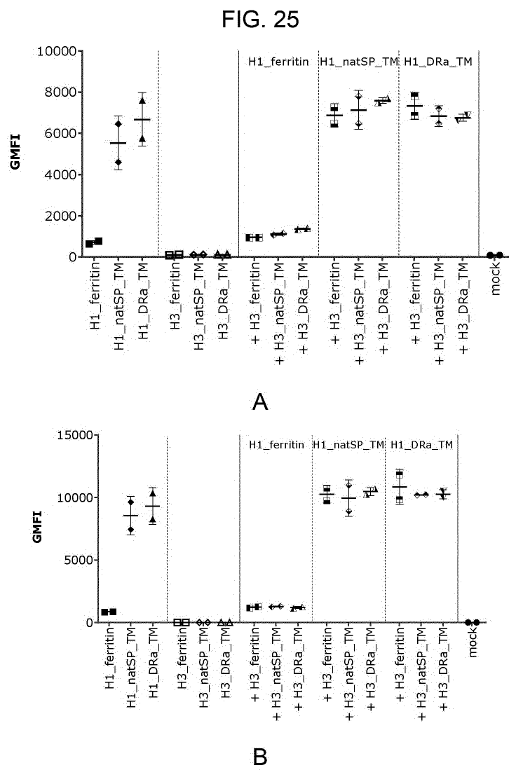

Results are shown on FIGS. 25A (cellular expression, anti-group 1) and 25B (surface expression, anti-group 1), obtained by using CR6261 antibody. CR6261 is a human-derived monoclonal antibody that recognizes the HA-stem of Group 1 HAs. It binds a conformational epitope and is therefore sensitive to the correct folding of the HA stem (Friesen, 2010).

High levels are comparable in single expression and H3 co-expression samples. Design of H3 has no effect on H1 translocation to the cell membrane.

### Example 13—In Vitro Detection of H3 in H3-TM/H3-F Transfected Cells

293T cells were seeded at a density of 2×10⁵ cells/well in 1 ml medium in a 24-well plate. The next day, cells were transfected with Lipofectamine formulated mRNA (“RNA”) or LNP-formulated mRNA (“LNP”). For Lipofectamine transfection, 1 μg mRNA (or water=mock) was mixed with 1.5 μl Lipofectamine 2000 (1:1.5 ratio) in a total of 250 μl Opti-MEM media and added to the cells. For LNP transfection, 1 μg LNP (or water=mock) was diluted in 50 μl growth media (DMEM+10% FCS+1% L-Glu+1% Pen/Strep) and added to the cells.

The next day, cells were washed in PBS and lysed within the plate using 200 μl RIPA buffer per well. Plates were incubated on ice for 30 min with gentle agitation. Lysates were transferred to Eppendorf tubes and centrifuged for 10 min, 4° C. Lysates were mixed with protein sample buffer, boiled for 5 min, and separated by SDS-PAGE using Mini Protean TGX 4-20% gradient gels. Samples were transferred to PVDF membranes by Western blot and blocked using Intercept blocking buffer in TBS for 1 h at room temperature.

The primary antibody for detection was pooled mouse serum from study 59-36-149 (evaluation of H1 and H3 protein designs) group 4 (immunized with H3_ferritin) diluted 1:500 in Intercept blocking buffer in TBS+0.2% Tween-20. Membranes were incubated with primary antibody solution over night at 4° C., rotating). The next day, membranes were washed 3×10 min in TBS+0.1% Tween-20 and incubated with secondary antibody IRDye® 680RD-conjugated goat anti-mouse IgG antibody diluted 1:10,000 in Intercept blocking buffer in TBS+0.2% Tween-20 for 1 h at room temperature. Membranes were washed 3×10 min in TBS+0.1% Tween-20 and bands were visualized using the Odyssey CLx image system.

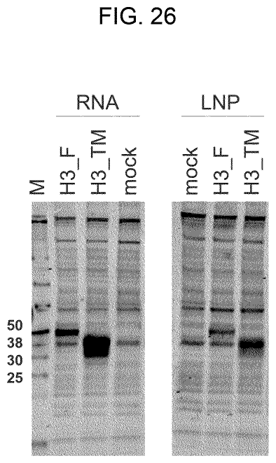

Results are shown in FIG. 26. Immunoblot detection of H3-stem confirms lower overall expression of ferritin construct in comparison to TM, similar to what was seen by flow cytometry. Mouse serum after H3-ferritin vaccination was used for detection (H1/H3 immunogenicity study, same serum as in example 19).

### Example 14—In Vitro Immune Stimulation of H1/H3-LNPs

Human peripheral blood mononuclear cells (PBMC) were isolated from whole blood of anonymous donors by Ficoll paque density gradient centrifugation, washed with PBS, and cultured in RPMI 1640+1% L-Glu+1% Pen/Strep+10% FCS. For stimulation with LNPs, PBMC from 4 independent donors were incubated in triplicate with LNP samples. 4×10⁵ cells/well were seeded into a 96-well plate and incubated with 10 μg/ml mRNA/LNP in a total of 0.2 ml. Samples representing 2 mRNAs were treated with LNPs, in which both mRNAs are formulated together in a 1:1 molar ratio.

After 24 h supernatants were collected and analyzed by human IFN-α ELISA according to manufacturer's instructions (human pan IFN-α ELISA kit from PBL). Cell supernatants were diluted 1:20 or 1:40 depending on the human donor before they were added to the ELISA plates. The assay is designed as a sandwich ELISA, where an anti-IFNα antibody is coated to the plate. Then, tissue culture supernatant is added to the plate and IFNα would be bound by the coated antibody. Supernatants are removed, plates are washed and incubated with a biotin-conjugated anti-IFNa-antibody, followed by HRP-conjugated streptavidin. The ELISA is developed using TMB substrate, stopped and the absorbance is read at 450 nm using the Synergy HTX plate reader. An IFNα standard is provided by the kit, which is run in parallel to the samples, allowing for quantification of protein concentration within the range of diluted standard samples.

The technical controls in this assay are comprised of two LNP-formulated mRNAs encoding for the rabies virus glycoprotein (CV7202 and R1803), a TLR7/8 agonist (ssRNA40), and medium as a negative control. CV7202 and R1803 have been produced in different production lines and are known to induce different levels of IFNα from human PBMC.

To better compare data from different donors, the results are normalized as follows. First, the mean IFNα concentration of each sample is calculated from the triplicate quantification. Then, the IFNα value for sample “CV7202 GMP” is set to 100% and the results for all other samples from the same donor are normalized to this sample, i.e. [mean IFNα concentration sample]/[mean IFNα concentration CV7202]*100=[% of CV7202]. Graph depicts mean+/−SD.

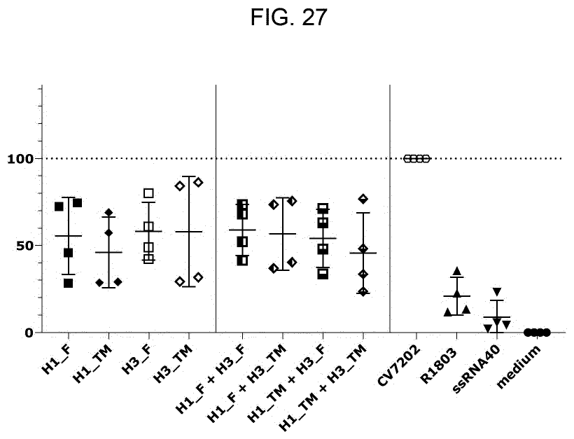

Results are shown on FIG. 27 and represent in vitro IFNα stimulation from hPBMC (from 4 donors normalized to CV7202). Equal amounts of each LNP were tested for IFNα induction in human PMBC. All mono-/bivalent vaccine candidates induced similar levels of IFNα, below those induced by a comparator LNP-formulated mRNA known to induce high levels of IFNα in this assay.

### Example 15—In Vivo Serum IFNα Levels, 18 Hours Post Prime Immunization

Mouse Immunization

The immunogenicity of H1 and H3 stem mRNA constructs was evaluated in BALB/c mice. Combinations of mRNAs were mixed at equimolar ratios and co-formulated into a single LNP. Ten female BALB/c mice were immunized at Day 0 and Day 21 with:
(a) H1-stem ferritin encoding mRNA construct
(b) H1-stem TM encoding mRNA construct
(c) H3-stem ferritin encoding mRNA construct
(d) H3-stem TM encoding mRNA construct
(e) H1-stem ferritin and H3-stem ferritin encoding mRNA constructs
(f) H1-stem ferritin and H3-stem TM encoding mRNA constructs
(g) H1-stem TM and H3-stem ferritin encoding mRNA constructs
(h) H1-stem TM and H3-stem TM encoding mRNA constructs
(i) QIV (commercially available quadrivalent influenza vaccine comprising inactivated split influenza virions of the strains A/Guangdong-Maonan/SWL1536/2019 (H1N1) pdm09, A/Hong Kong/2671/2019 (H3N2), B/Washington/02/2019 (B/Victoria) and B/Phuket/3073/2013 (B/Yamagata) without adjuvant (only 6 mice for this group)
(j) NaCl (only 6 mice for this group).

The mouse immunization protocol is further applicable for Examples 16 to 21.

Assay, Analysis and Results

Blood samples were taken 18 h after the first immunization by retrobulbar bleeding. 140 μl blood were collected into Z-clot activator 200 μl microtube (Sarstedt, Cat #20.1291) and incubated at room temperature (RT) for 30 min to allow for clotting. Samples were centrifuged for 5 min, 10 000 rcf, RT and serum was transferred to fresh Eppendorf tubes and stored at −20° C. Mouse IFN-α was quantified using a mouse IFN-α ELISA according to manufacturer's instructions (VeriKine-HS Mouse Interferon Alpha All Subtypes frpm PBL). Sera was diluted 1:20 and 100 μl of dilution were tested. The assay uses 96 well plates coated with anti-murine IFNα antibody. Serum is added to the plates and mlFNa is bound by the antibody on the plate.

Serum is removed, plates are washed briefly and incubated with an anti-murine IFNα detection antibody, which is biotin-conjugated and binds a different epitope on IFNα than the coated antibody. This sandwich is then detected with HRP-conjugated streptavidin and visualized using TMB (colorimetric ELISA substrate). Plates are stopped with H2SO4 stopping solution and absorbance is read at 450 nm using the Synergy HTX reader. An IFNα standard is provided by the kit, which is run in parallel to the samples, allowing for quantification of protein concentration within the range of diluted standard samples. The graph depicts mean+/−SD.

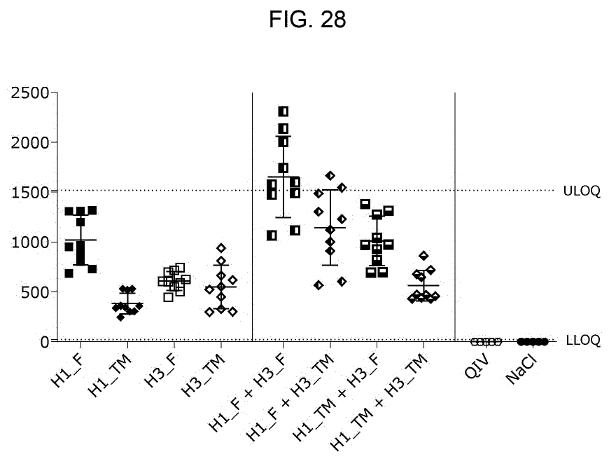

Results are shown in FIG. 28. The results are expressed as IFNα in pg/ml. In mice, ferritin constructs are more immune stimulating.

### Example 16—In Vivo T Cell Responses CD4⁺IFNγ⁺TNF⁺, at Day 35

For isolation of splenocytes, spleens are handled in PBS+1% FCS and ground using the plunger of a sterile 5-10 ml syringe. Cells are passed twice through a cell strainer with 0.45 μm pore size and pelleted by centrifugation. To remove erythrocytes, cells are incubated with red blood cell lysis buffer (144 mM NH₄Cl, 17 mM Tris) for up to 10 min at room temperature. Samples are centrifugated and immediately washed twice with PBS+1% FCS and frozen until further use or used directly for intracellular cytokine staining.

For intracellular cytokine staining, cells were resuspended in aMEM complete media (aMEM+10% FCS+1% Glutamine+1% Pen/Strep+10 mM Hepes) and stimulated in 96 well round bottom plates using 2×106 cells per well. After seeding, cells are pelleted by centrifugation of the plates and the supernatant is removed by inversion. Cells are resuspended in media containing the following stimuli:
1 μg/ml peptide library (covering either H1-stem or H3-stem as indicated in the graph)
2.5 μg/ml anti-CD28 antibody
PE-Cy7-conjugated anti-CD107a antibody (1:100 dilution)

Cells were incubated for 1 h at 37° C. before addition of GolgiPlug. After an additional 5-6 h, the media was replaced with fresh αMEM complete media and plates were stored a 4° C. over night.

The next day, cells were washed twice with PBS and stained with Aqua-Dye (1:1000 in PBS; 30 min at 4° C.) for differentiation of live and dead cells. Cells were washed 2× with PBS+0.5% BSA and then stained with anti-surface marker antibodies for 30 min at 4° C. The staining solution contained α-CD8-APC-Cy7 (1:200), α-CD4-BD-Horizon V450 (1:200), α-Thy1.2-FITC (1:200)+FcγR-block (1:100) in 100 μl PBS/0.5% BSA. Cells were washed again with PBS+0.5% BSA and treated with Cytofix/Cytoperm for intracellular staining (20 min, room temperature). Cells were washed 2× with Permwash and stained for cytokines using α-TNF-PE (1:100)+α-IFNγ-APC (1:100) in 100 μl PermWash (30 min at 4° C.). Cells were washed 2× more in Permwash, resuspended in PBEA, and measured on a ZE5 flow cytometer. Results are analzed using FlowJo.

Graphs indicate IFNγ/TNF double positive CD4+ T cells (% of CD4+ cells) specific for H1-stem (H1N1 A/Michigan/45/2015) or H3-stem (H3N2 A/Finland/486/2004). Lines indicate mean+/−SD.

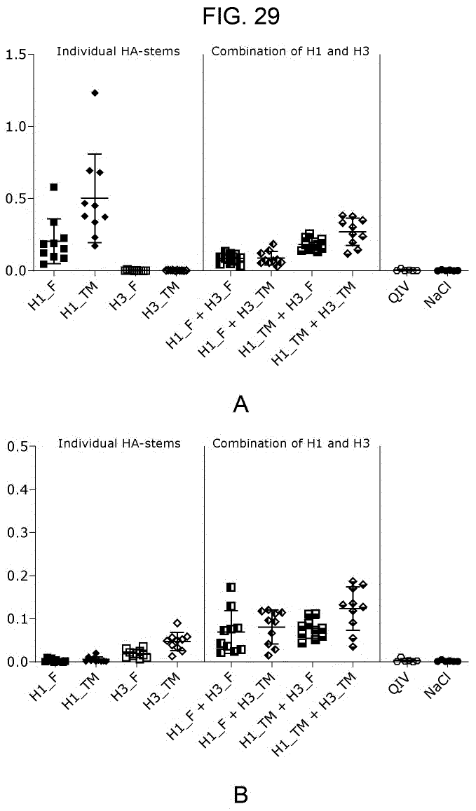

Results are show on FIGS. 29A and 29B. Both antigens, in both protein designs induce specific CD4+ T cells. Levels induced by TM trend to be higher than those induced by ferritin design.

### Example 17—In Vivo T Cell Responses CD8⁺IFNγ⁺TNF⁺, at Day 35

The assay protocol is the same as described in Example 16. Graph depicts IFNγ/TNF double positive CD8+ T cells (% of CD8+ cells), lines indicate mean+/−SD.

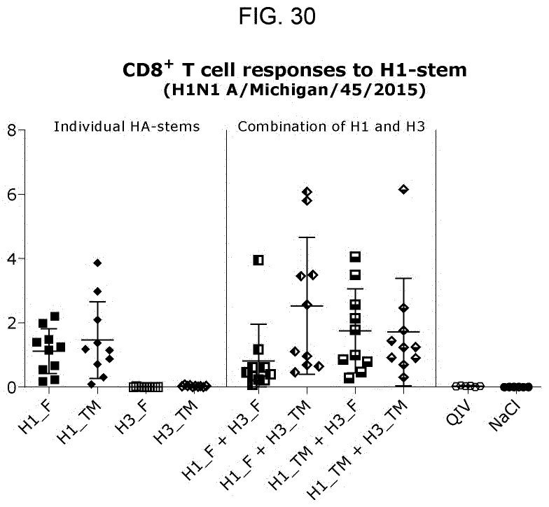

Results are shown in FIG. 30. H1-stem designs efficiently induce CD8⁺. No clear difference between the protein designs can be observed.

### Example 18—In Vivo T Cell Responses CD8⁺IFNγ⁺CD107⁺, at Day 35

The assay protocol is the same as described in Example 16. Graph depicts IFNγ/CD107 double positive CD8+ T cells, lines indicate mean+/−SD.

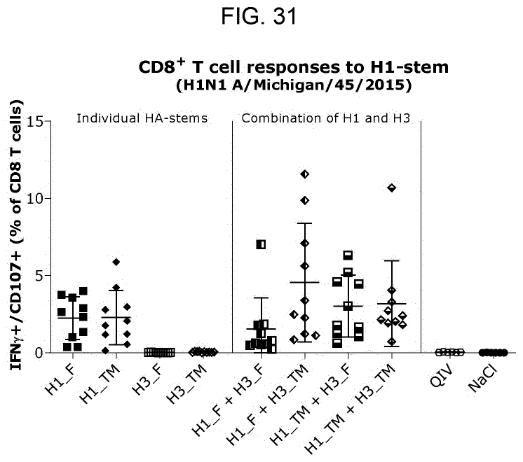

Results are shown in FIG. 31. CD8+ T cells are multifunctional expressing IFNγ, TNF and/or CD107.

### Example 19—In Vivo Anti-H1 Binding Antibodies, at Day 21

Serum samples were taken by retrobulbar bleeding 21 days after the first immunization. Serum was prepared as described in Example 15.

Black bottom 96 well ELISA plates were coated with recombinant HA (A/Hawaii/70/2019 (H1N1)) using 100 μl of a 1 μg/ml dilution in Bicarbonate buffer for 4-5 h at 37° C. Plates were washed with PBS/0.05% Tween 20 and blocked over night with blocking buffer (5% milk in PBS/0.05% Tween 20) at 4° C. The next day, ELISA plates were washed and incubated with serum dilutions (10-fold dilutions in blocking buffer, starting at 1:50, using 100 μl/well) and incubated for 2-4 h at room temperature. Plates were washed three times with PBS/0.05% Tween 20. HRP-conjugated anti-mouse total-IgG was diluted 1:5,000 in blocking buffer and incubated for 1 h at room temperature. Plates were washed four times and developed with Amplex UltraRed. The endpoint titer represents the reciprocal value of the last serum dilution with a signal above the cutoff. The cutoff for a positive signal was defined as the mean+5× the standard deviation of background wells (without addition of serum).

Lines represent GMT+95% confidence interval.

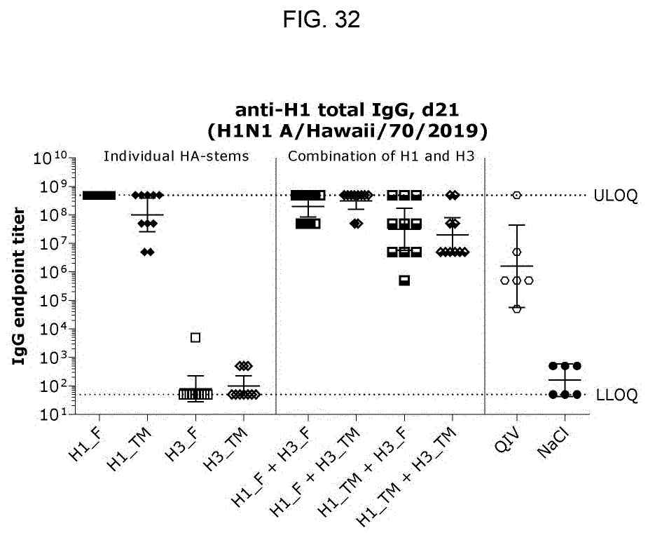

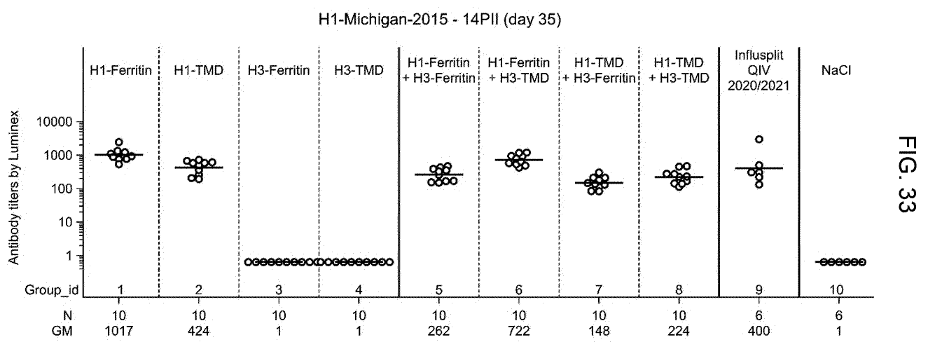

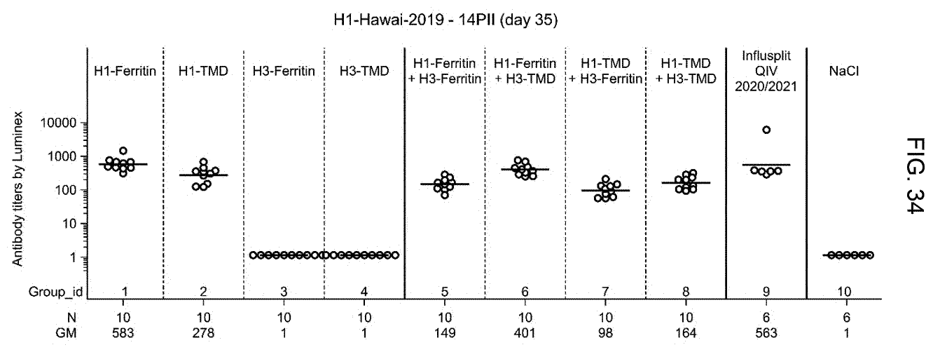

Results are shown in FIG. 32. A single dose of mRNA/LNP vaccine is sufficient to induce heterologous antibody responses. Ferritin design induces higher titers than TM.

### Example 20—In Vivo Anti-HA IgG Antibodies by Multiplexing Serology Luminex at 14 Days Post Dose 2

Anti-HA IgG Multiplexing Serology by Luminex

Fourteen different populations of fluorescent magnetic beads (with antigen-specific levels of APC/APC-Cy7 fluorescence) were coated in house using the following method. One million of each bead population was added to a tube, washed and resuspended with NaH₂PO₄ 100 mM buffer before activation of carboxyles fragments by addition of Sulfo-NHS (N-hydroxysulfosuccinimide/ThermoFischerScientific cat. A39269 at 50 mg/ml) and EDC (1-ethyl-3-(3-dimethylaminopropyl) carbodiimide hydrochloride/ThermoFischerScientific cat. A35391 at 50 mg/ml) and incubation 20 min at RT on rotative agitator. After each step, beads were vigorously vortexed and sonicated. Beads were washed with PBS and coated with a fixed amount of 14 recombinant hemagglutinin (HA) antigens (10 or 20 μg depending on the antigen, to have the optimal signal). See the table 1 for description of the antigens. The beads were incubated 2 hours at RT on rotative agitator. Beads were washed with PBS-TBN buffer (PBS-0.1% BSA-0.02% Tween 20-0.05% Azide pH7.4) and incubated with this buffer 30 min at RT on rotative agitator. The beads were than washed again and resuspended with PBS-TBN buffer. The beads were counted using a TC20 Biorad counter and stored in 4° C.

The assay was performed as follows. Serial dilutions of sera in PBS-Tween 0.05% buffer were prepared in a 96-wells plate (volume of 50 μl/well). 50 μl of bead mix (containing 500 beads of each beads population) were then added to each well and incubated 60 min at RT under gentle shaking. The beads were washed with PBS-Tween 0.05% on a magnetic plate washer, and 50 μl of anti-mouse IgG PE labeled antibody (Southern Biotech cat. 1030-09S) diluted 1:50 in PBS-Tween 0.05% were added to each well and incubated 60 min at RT under gentle shaking. The beads were washed with PBS-Tween 0.05% on a magnetic plate washer and resuspended in 100 μl of PBS-Tween 0.05%, before the acquisition on Luminex Bioplex 200 reader (Biorad). The antibody titers were calculated using Softmaxpro (Molecular Devices) software.

TABLE 1
HA antigens included in the Multiplexing Serology Panel Luminex

| HA group | Subtype | strain |
|---|---|---|
| A1 | H1 | A/Michigan/45/2015 |
| | | A/Hawaii/70/2019 (Season 2020/2021) |
| | | A/Christchurch/16/2010 |
| | | A/California/6/09 |
| | H2 | A/Singapore/1/57 |
| | H5 | A/Vietnam/1203/2004 |
| A2 | H3 | A/Finland/486/2004 |
| | | A/Hong Kong/45/2019 (Season 2020/2021) |
| | | A/Perth/16/2009 |
| | | A/Beijing/47/1992 |
| | | A/Philippines/2/1982 |
| | | A/Hong Kong/1/68 |
| | H7 | A/Shanghai/2/2013 (stem construct), identical HA |
| | | polypeptide sequence as A/Anhui/1/2013 |
| | H10 | A/Jiangxi-Donghu/346/2013 (stem construct) |

Analysis and Results

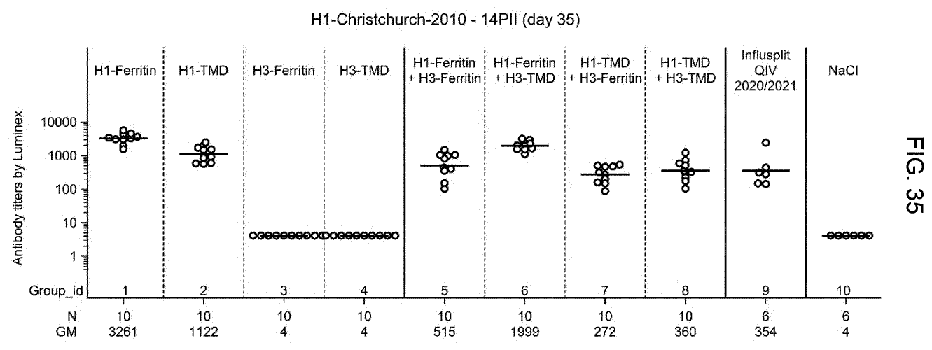

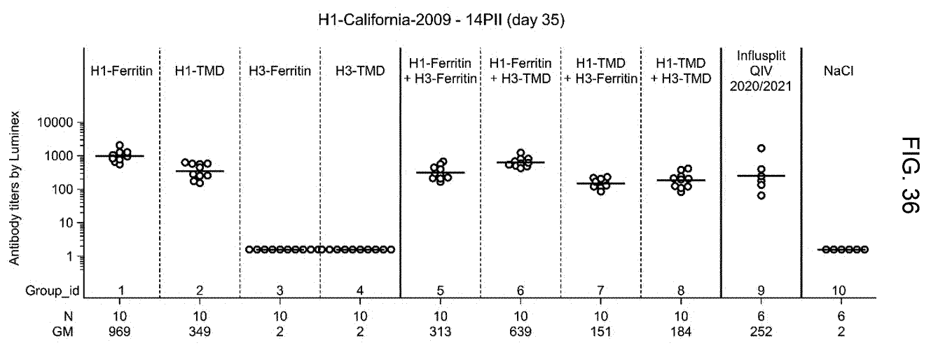

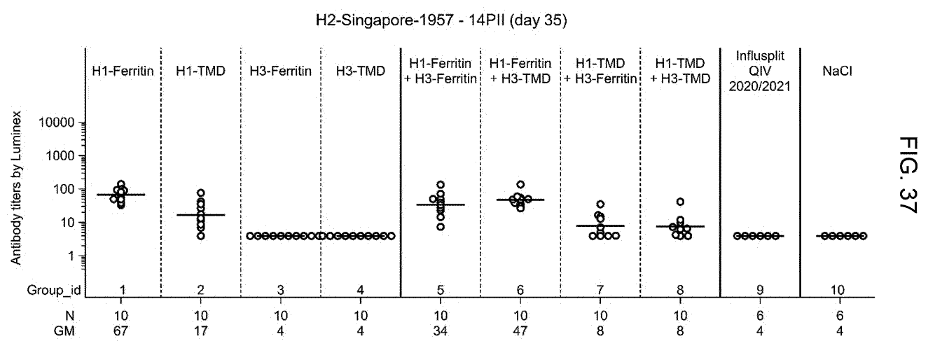

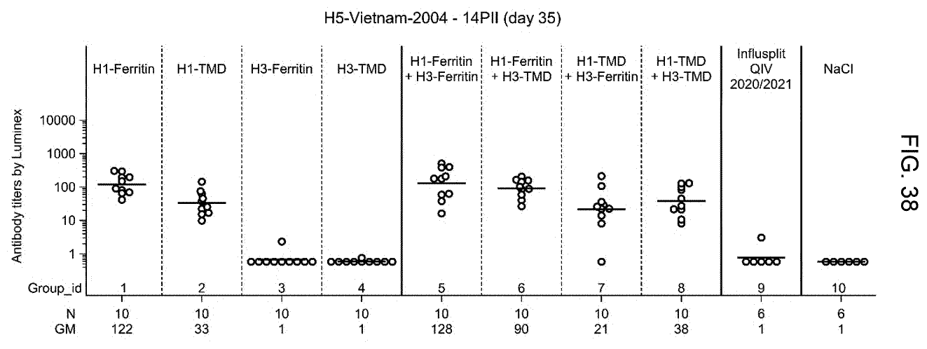

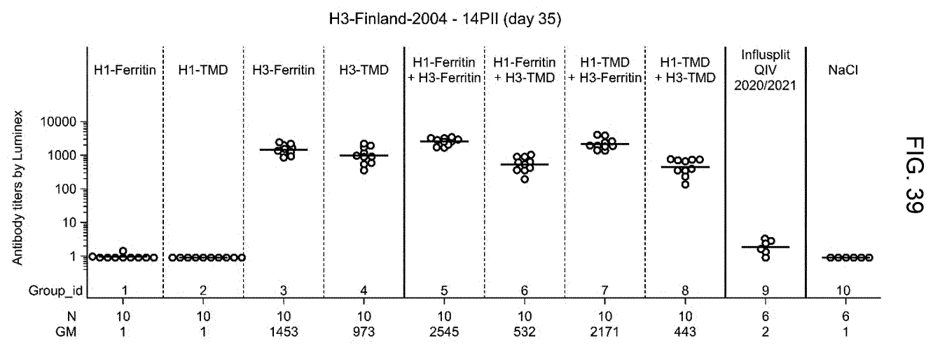

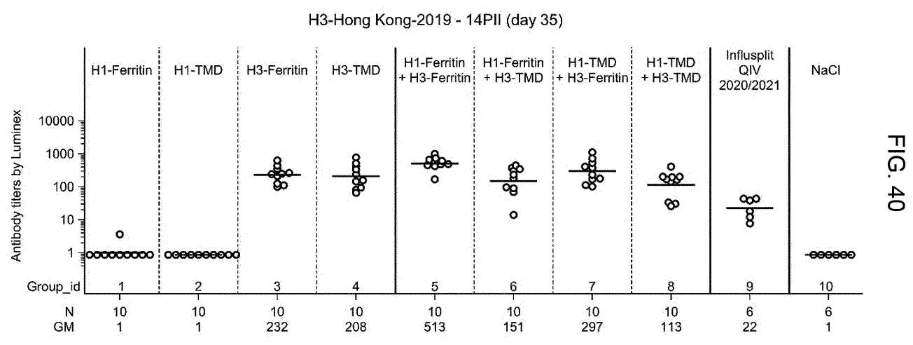

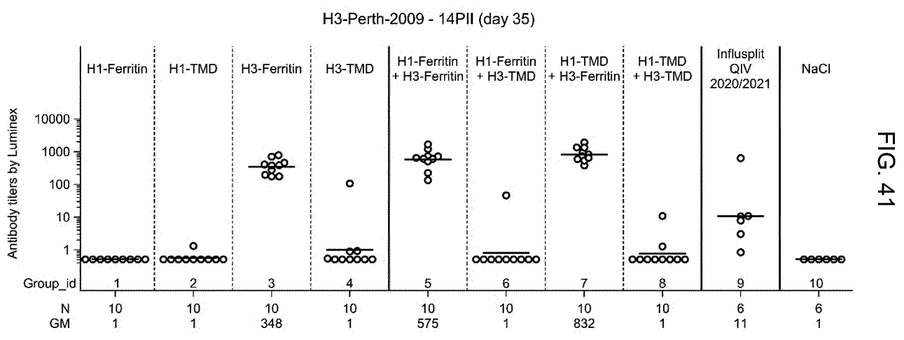

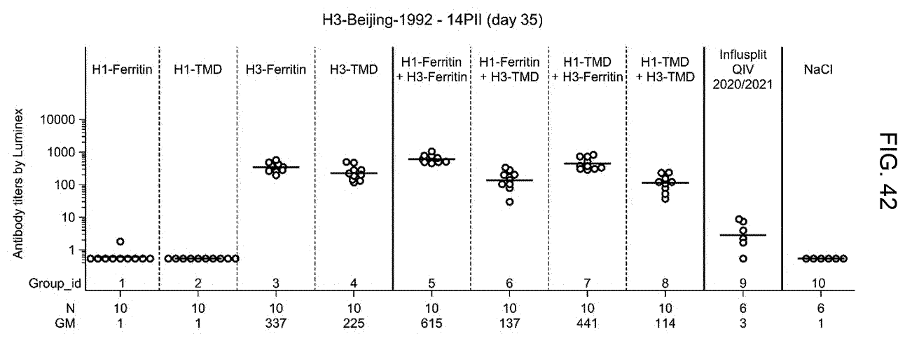

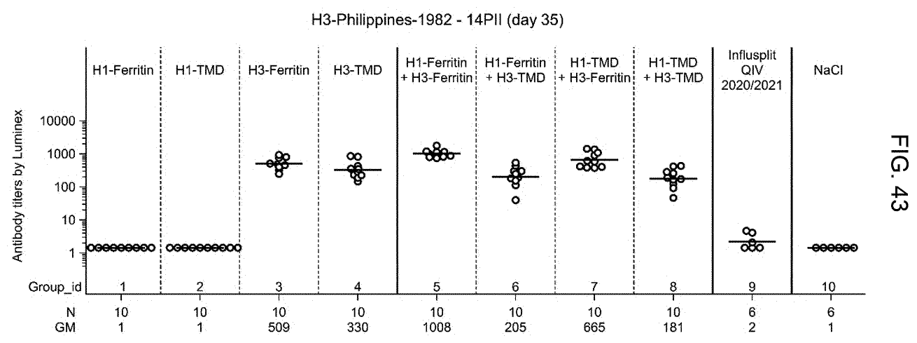

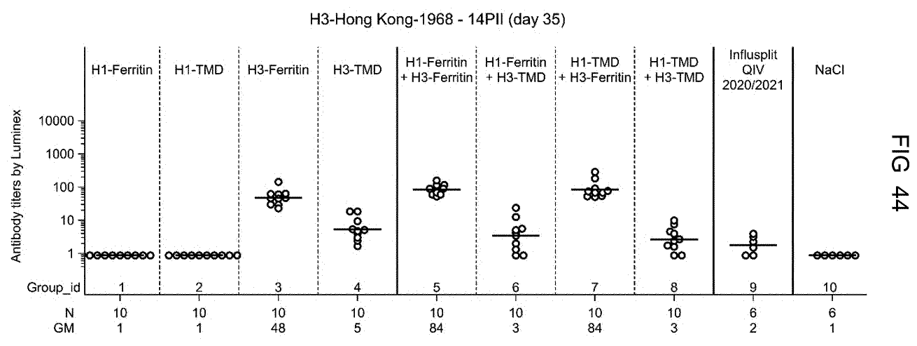

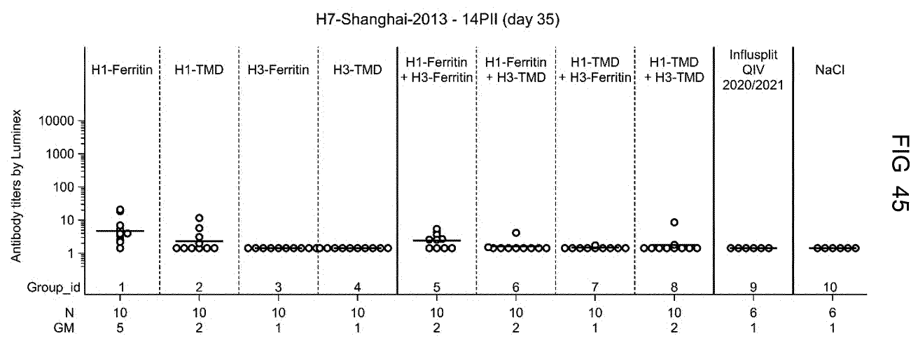

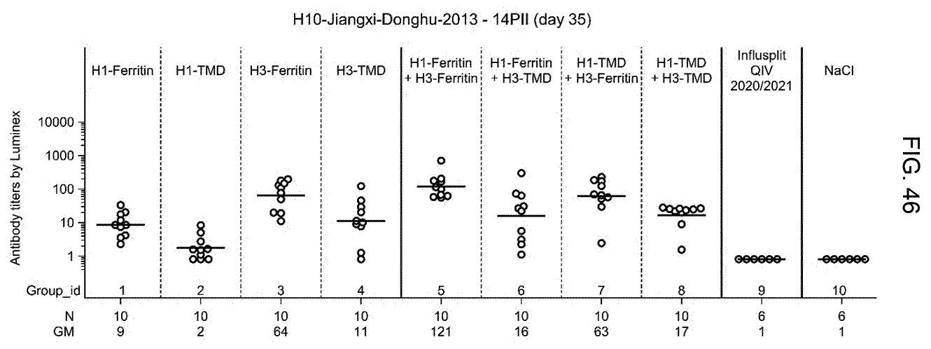

Titers in IgG antibodies binding to 14 different HAs from group A were measured by Multiplexing Luminex assay at 14 days post second immunization (day 35). Results are shown in FIG. 33-46. Individual titers values are plotted, with the geometric mean (GM) and sample size per group (N).

H1-stem ferritin and TM antigens were immunogenic inducing both homologous responses (against A/Michigan/45/2015), heterologous responses (against other H1 antigens) and heterosubtypic cross-reactive responses (against H2 and H5 antigens).

H3-stem ferritin and TM were immunogenic inducing both homologous responses (against A/Finland/486/2004), heterologous responses (against other H3 antigens). H3-stem ferritin and TM antigens induced heterosubtypic cross-reactive responses (against H10 A/Jiangxi-Dongu/346/2013). H1-stem ferritin and TM antigens induced cross-reactive responses against A2 group HA antigens (i.e., H7 A/Shanghai/2/2013) with higher responses observed for H1-stem ferritin.

The combination of both H1 and H3 antigens induced broad cross reactivity across group A1 and A2. Altogether, the different analyses comparing the 4 combination groups indicated that H1 ferritin+H3 ferritin induced the broadest antibody response across the 14 HA tested.

### Example 21—In Vivo Anti-H1 A/Michigan/45/2015 Stem Antibodies by ADCC Reporter Bioassay at 14 Days Post Dose 2

Antibody Dependent Cell Cytotoxicity (ADCC) Reporter Bioassay (Promega)

For determination of ADCC functionality, the mouse FcγRIV kit from Promega was used, with the following protocol. Serial dilutions of sera were prepared in 96-wells plates. Target cells (Expi293 cells transfected in house to express hemagglutinin stem antigen from H1 A/Michigan/45/2015 on their surfaces) were added to each well (24000 cells/well). Effector cells (Jurkat cells, from the kit, transfected with an enzymatic pathway inducing bioluminescence when activated by antigen-antibody-FcγRIII complex) were also added to each well (60000 cells/well), and incubated 6 hours at 37° C. Luciferase activity was then measured, after having applied the Bio-Glow substrate (provided in the Kit), using a Luminescence plate reader. Results were expressed as Area Under the Curve (AUC).

Analysis and Results

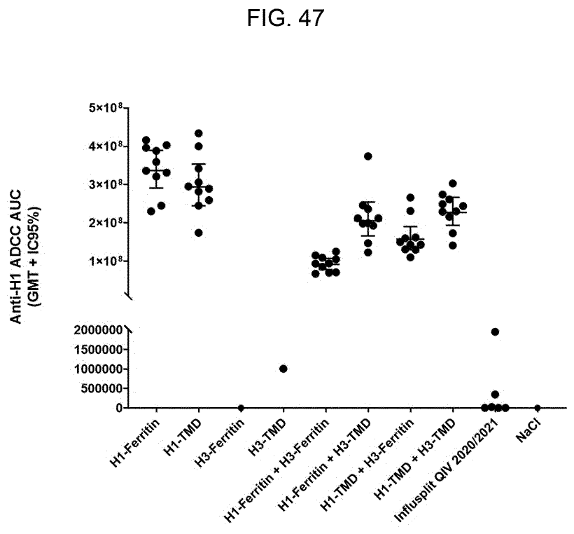

ADCC functional antibodies against A/Michigan/45/2015 H1 stem were measured by ADCC Reporter Bioassay Promega at 14 days post second immunization (day 35). Results are shown in FIG. 47. Individual AUC (Area under the curve) values are plotted, with the geometric mean (GMT) and 95% confidence interval. For H3-ferritin and H3-TMD groups, only one pooled sample of the group was tested.

The antibodies elicited by all the tested constructs containing H1 stem antigen were functional by ADCC.

### Example 22—In Vitro Anti-H3 Stem Antibodies by ADCC Reporter Bioassay

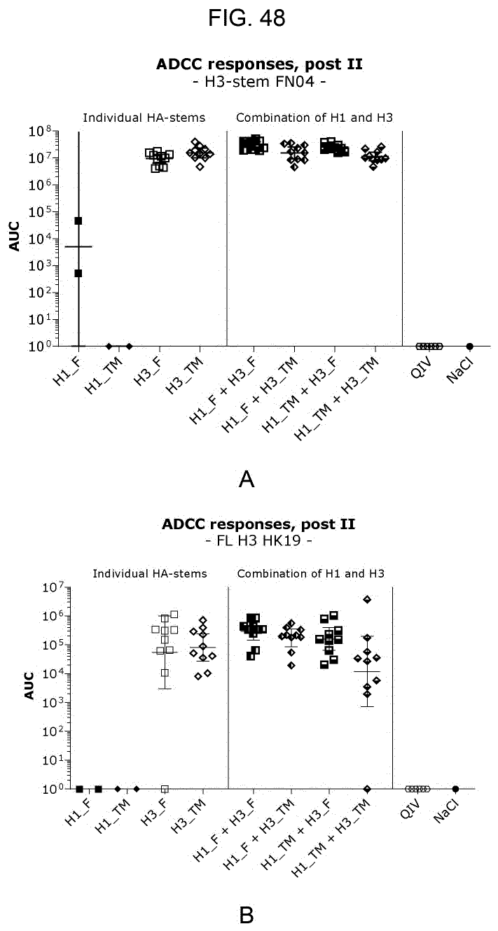

For H3-specific ADCC assays, target cells were prepared by transfection. HeLa cells were seeded into white flat bottom 96-well plate at a density of 10,000 cells in 200 μl medium per well. The next day, cells were transfected with mRNA encoding the respective target protein using Lipofectamine 2000 according to manufacturer's instructions. For each well, 0.05 μg of mRNA were mixed with 0.075 μl Lipofectamine 2000 (1:1.5 ratio) in a total of 50 μl Opti-MEM media and added to the cells. MRNAs encoded either the membrane-bound H3-stem portion of H3N2 A/Finland/486/2004 (i.e., H3_TM vaccine; FIG. 48A) or the full length/wild-type H3 of H3N2 A/HongKong/45/2019 (contained in rec. HA vaccines for 2020/2021; FIG. 48B).

After 18-24 h cells were used in mFcγRIV ADCC Reporter Bioassay (Promega) according to manufacturer's instructions. First the medium is replaced with 25 μl assay buffer/well. Serum samples are diluted three-fold (ten times) in assay buffer starting at 1:33.3 (1:100 final dilution in well) and 25 μl of each dilution are added to a well containing target cells. Serum samples from groups where no ADCC activity was expected were pooled (2 pools of 5 animals each for H1_F and H1_TM groups, 1 pool of 6 animals for NaCl).

Murine FcγRIV effector cells (i.e., Jurkat cells stably expressing mFcγRIV and NFAT-response element dependent Luciferase expression cassette) are thawed in assay buffer at a concentration of 3×10⁶ cells/ml and 25 μl effector cell suspension (75,000 cells/well) was added to assay wells. Plates were incubated for 6 hours at 37° C., 5% CO₂ to allow for signalling and Luciferase expression to take place.

For detection, assay wells were incubated with 75 μl Bio-Glo™ Luciferase assay substrate for 15 min at room temperature and read using the BioTek Synergy HTX plate reader. Relative light units were plotted against the serum dilution and the area under the curve (AUC) was calculated using GraphPad Prism 9. Mean values+three standard deviations of wells incubated without serum were used as baseline value for AUC calculation. Graphs depict GMT+95% CI. For samples without a signal, AUC was set to 1.

Results are shown in FIGS. 48A and 48B. H3-stem vaccines induced ADCC antibodies, that target the homologous H3-stem (FIG. 48A). Antibodies can also bind to an heterologous full length HA from a different H3N2 strain (FIG. 48B).

Section 2.2—Examples with Modified Nucleosides

### Example 23—Innate Immune Stimulation In Vitro and In Vivo

In Vivo Study—Mouse Immunization

A further study was conducted to investigate the impact of nucleosides modification (pseudouridine and 1-methyl-pseudouridine) on immunogenicity. MRNAs encoding for H1-stem and H3-stem and produced with the same nucleosides were mixed at an equimolar ration and coformulated together into one LNP. Ten female BALB/c mice were immunized at Day 0 and Day 21 with:
(a) H1-stem ferritin and H3-stem ferritin encoding mRNA constructs based on unmodified nucleosides
(b) H1-stem ferritin and H3-stem ferritin encoding mRNA constructs based on pseudouridine nucleosides
(c) H1—stem ferritin and H3-stem ferritin encoding mRNA constructs based on 1-methyl-pseudouridine nucleosides
(d) H1—stem TM and H3—stem TM encoding mRNA constructs based on unmodified nucleosides
(e) H1—stem TM and H3—stem TM encoding mRNA constructs based on pseudouridine nucleosides
(f) H1—stem TM and H3—stem TM encoding mRNA constructs based on 1-methyl-pseudouridine nucleosides
(g) NaCl (only 5 mice for this group)

The mouse immunization protocol is further applicable to Examples 24, 25 and 27 to 29.

Assay, Analysis and Results of the In Vivo Study

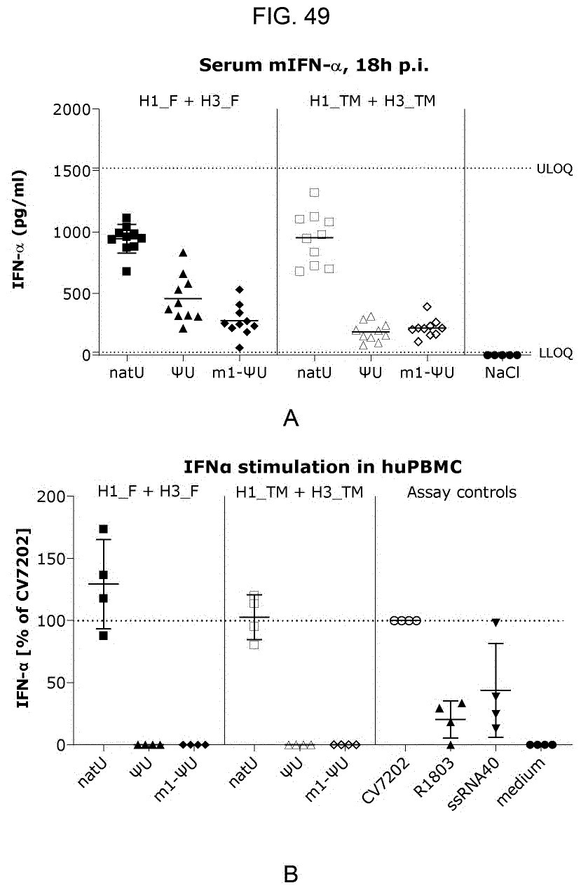

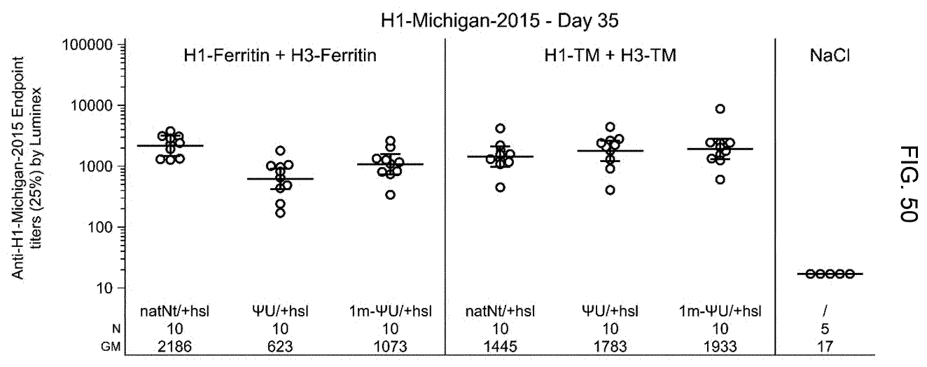

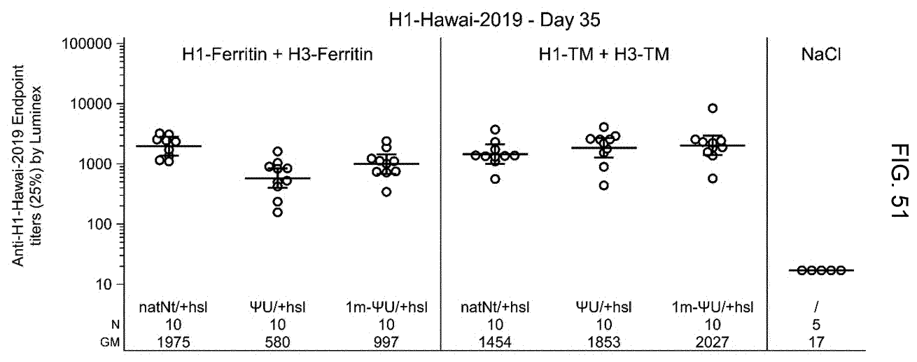

Mouse IFNα was detected in serum as described in Example 15. Lines indicate mean+/−SD. Results are shown in FIG. 49A. Nucleoside modifications reduce serum IFNα levels in response to immunization.

In Vitro Study

PBMC stimulation was performed as described in Example 14. Lines indicate mean+/−SD.

Results are shown in FIG. 49B. Nucleoside modifications reduce serum IFNα levels in response to stimulation.

### Example 24—In Vivo Anti-HA IgG Antibodies by Multiplexing Serology Luminex at 14 Days Post Dose 2 (with Modified Nucleosides)

Anti-HA IgG Multiplexing Serology by Luminex

The multiplexing Luminex assay was performed as described in Example 20.

Analysis and Results

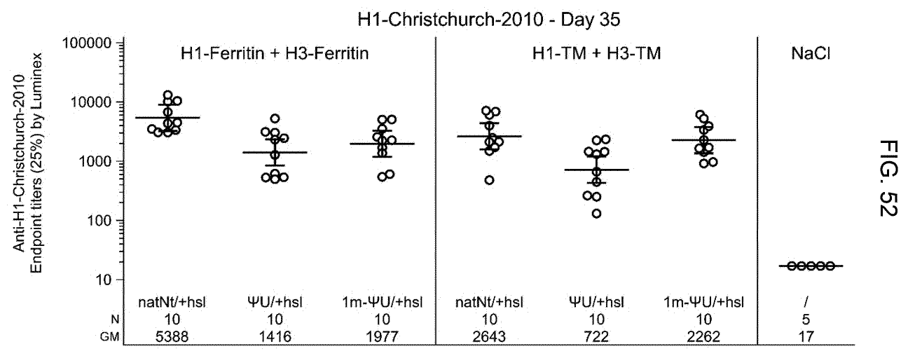

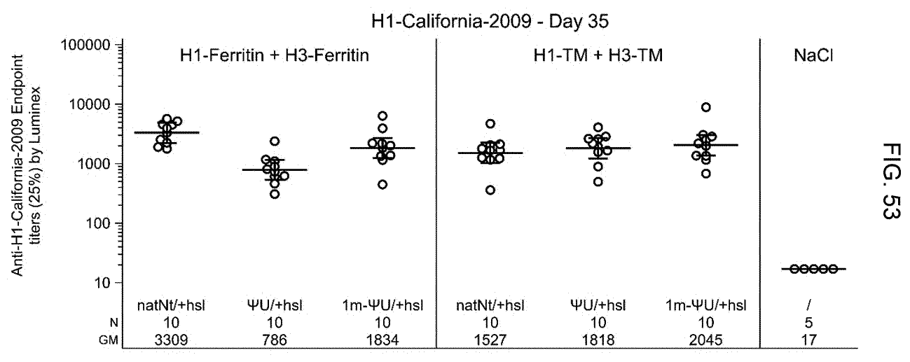

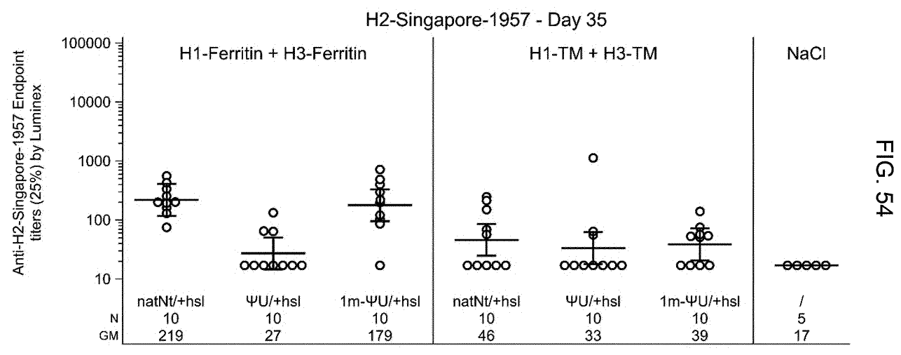

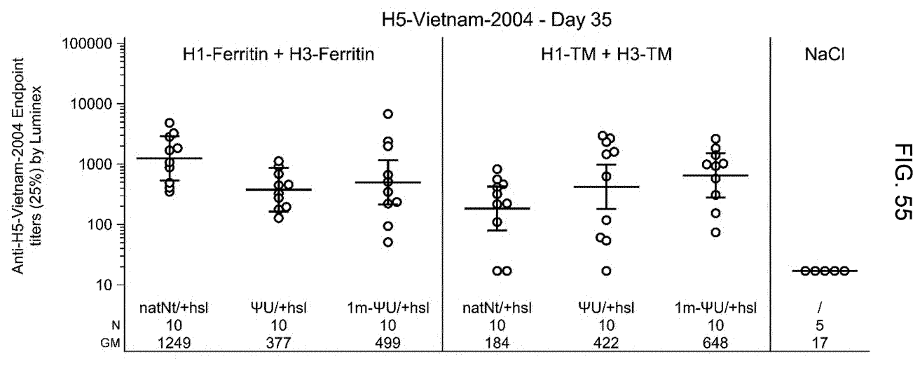

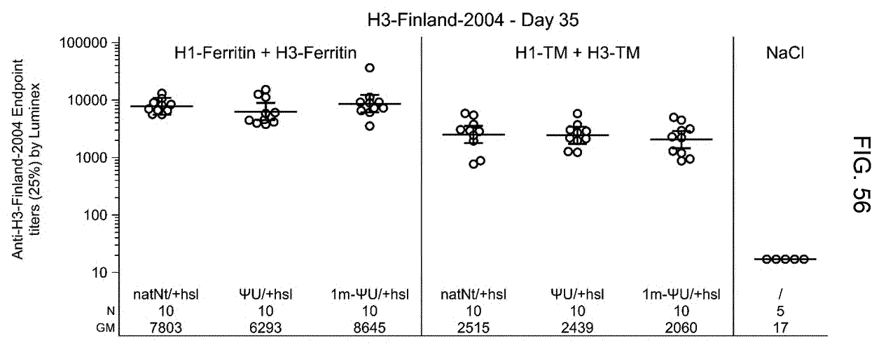

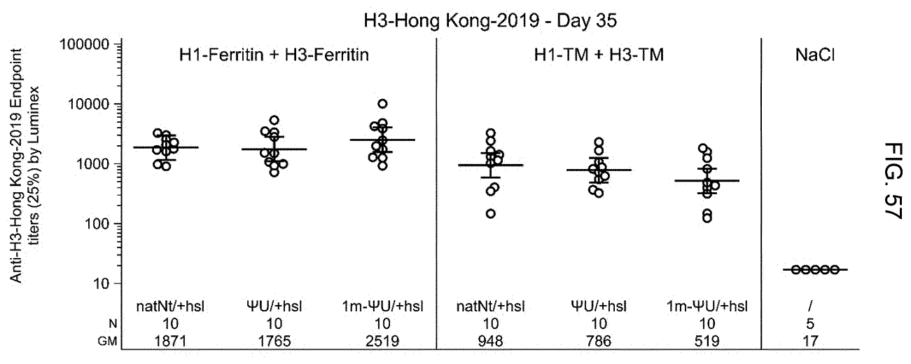

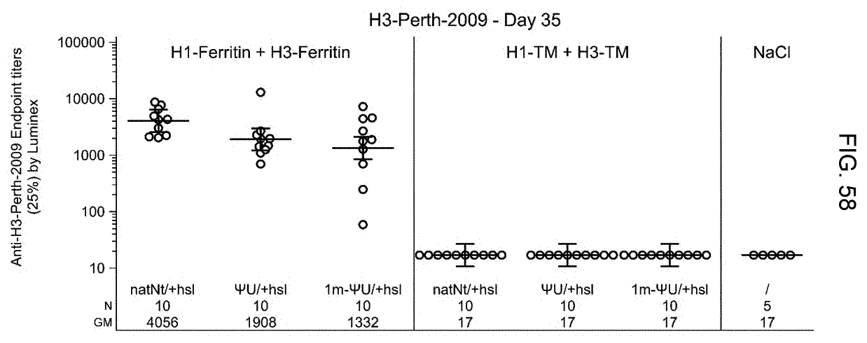

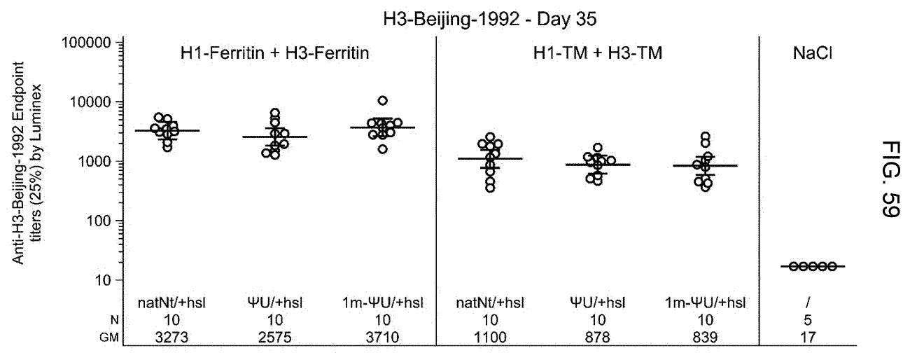

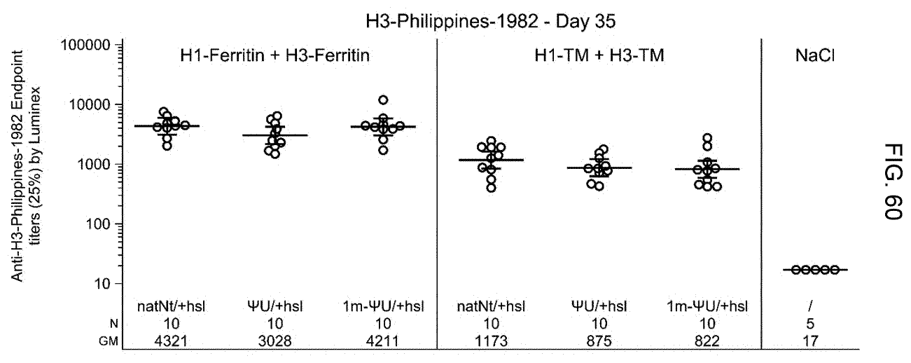

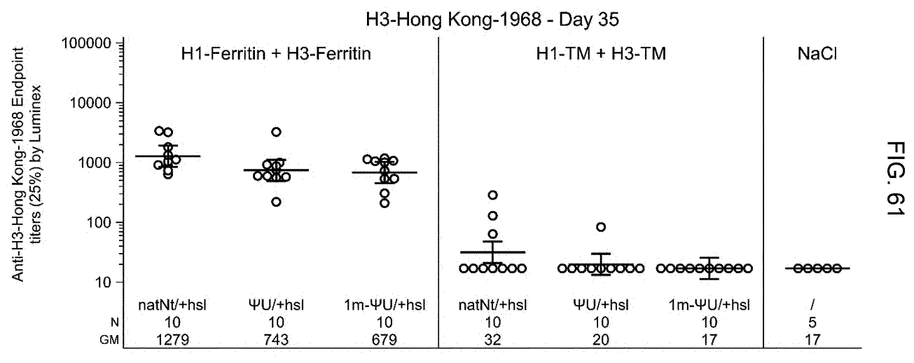

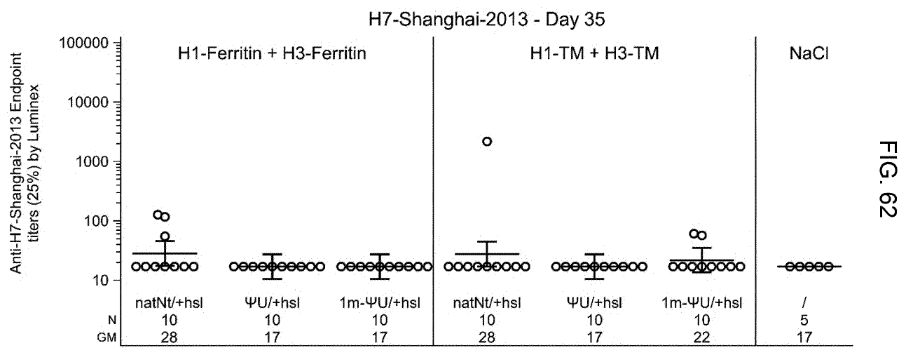

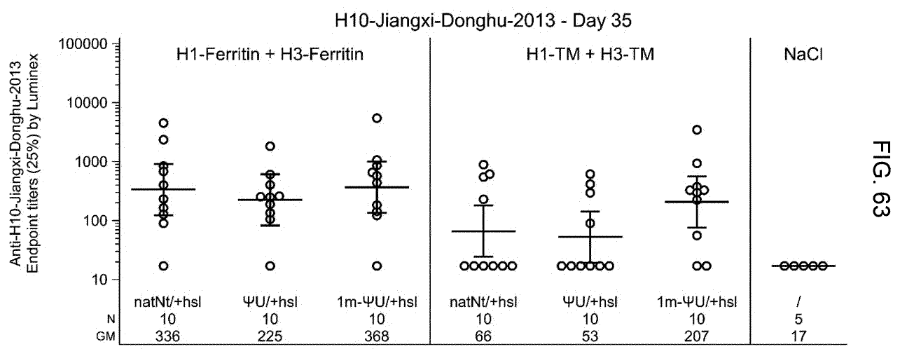

Titers in IgG antibodies binding to 14 different HAs from group A were measured by Multiplexing Luminex assay at 14 days post second immunization (day 35). Results are shown in FIG. 50-63. Individual titers values are plotted, with the geometric mean (GM), 95% confidence interval and sample size per group (N).

All H1-stem ferritin and TM antigens (unmodified- and modified nucleosides-based) were immunogenic, inducing homologous responses (against A/Michigan/45/2015), heterologous responses (against other H1 antigens) and heterosubtypic cross-reactive responses (against H2 and H5 antigens).

All H3-stem ferritin and TM antigens (unmodified- and modified nucleosides-based) were immunogenic, inducing homologous responses (against A/Finland/486/2004), heterologous responses (against other H3 antigens). All antigens induced heterosubtypic cross-reactive responses (against H10 A/Jiangxi-Dongu/346/2013).

The combination of both H1 and H3 antigens induced broad cross reactivity across group A1 and A2. Altogether, the different analyses comparing the different combination groups indicated that H1 ferritin+H3 ferritin induced the broadest antibody response across the 14 HA tested.

### Example 25—In Vivo Anti-H1 A/Michigan/45/2015 Stem Antibodies by ADCC Reporter Bioassay at 14 Days Post Dose 2 (with Modified Nucleosides)

Antibody Dependent Cell Cytotoxicity (ADCC) Reporter Bioassay (Promega)

The ADCC Reporter Bioassay was performed as described in Example 21.

Analysis and Results

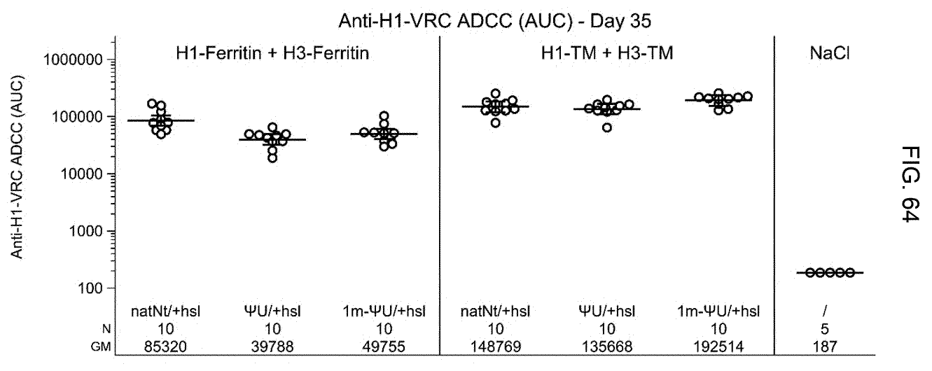

ADCC functional antibodies against A/Michigan/45/2015 H1 stem were measured by ADCC Reporter Bioassay Promega at 14 days post second immunization (day 35). Results are shown in FIG. 64. Individual AUC (Area under the curve) values are plotted, with the geometric mean (GMT) and 95% confidence interval.

The antibodies elicited by all the tested constructs containing H1 stem antigen were functional by ADCC.

### Example 26—In Vitro Anti-H3 A/Finland/486/2004 (H3N2) Stem Antibodies by ADCC Reporter Bioassay at 14 Days Post Dose 2

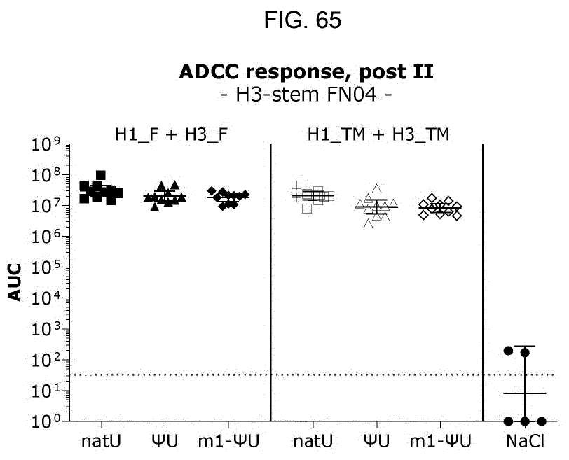

The H3-specific ADCC Reporter Bioassay was performed as described in Example 22, using target cells expressing the membrane-bound H3-stem portion of H3N2 A/Finland/486/2004 (i.e., H3_TM vaccine; FIG. 65).

Graph depicts individual area under the curve data (AUC) with GMT+95% CI indicated by lines.

Results are shown in FIG. 65. All vaccine candidates induced ADCC-inducing antibodies against H3.

### Example 27—In Vivo T Cell Responses CD4⁺IFNγ⁺TNF⁺, at Day 35 (Modified Nucleosides)

Splenocyte isolation and intracellular cytokine staining were performed as described in Example 16. Graph depicts IFNγ/TNF double positive CD4+ T cells, lines indicate mean+/−SD.

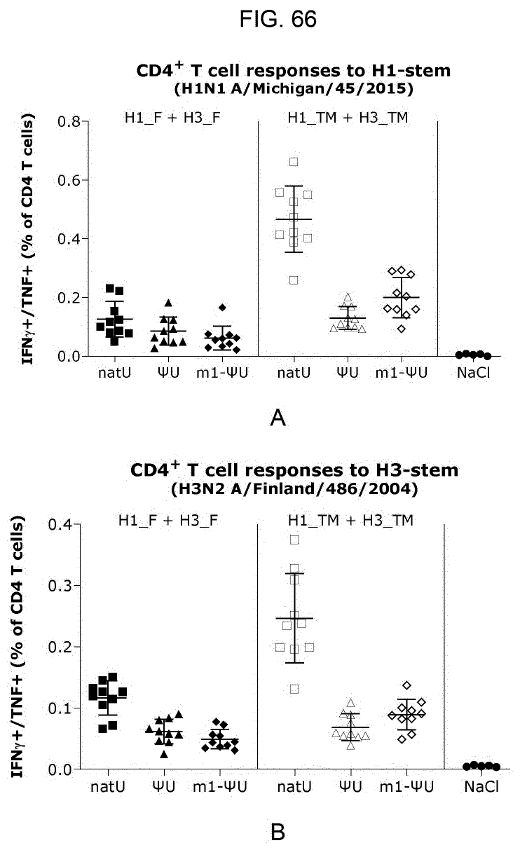

Results are shown in FIGS. 66A and 66B. TM designs induce higher level of HA-stem specific antibodies.

### Example 28—In Vivo T Cell Responses CD8⁺IFNγ⁺TNF⁺, at Day 35 (Modified Nucleosides)

The assay protocol is the same as described in Example 16.

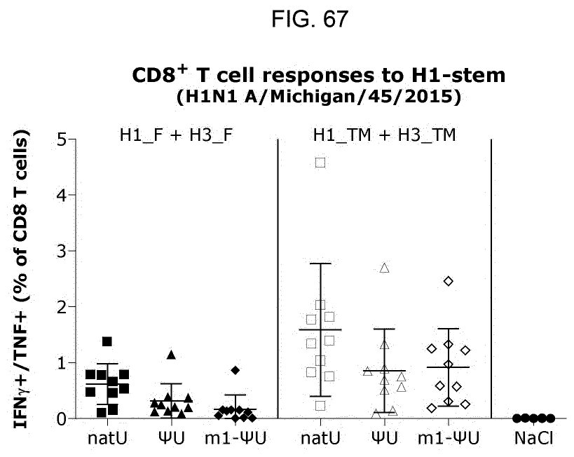

Graph depicts IFNγ/TNF double positive CD8+ T cells, lines indicate mean+/−SD. Results are shown in FIG. 67. TM designs induce higher levels of HA-stem specific CD8⁺ T cells.

### Example 29—In Vivo T Cell Responses CD8⁺IFNγ⁺CD107⁺, at Day 35

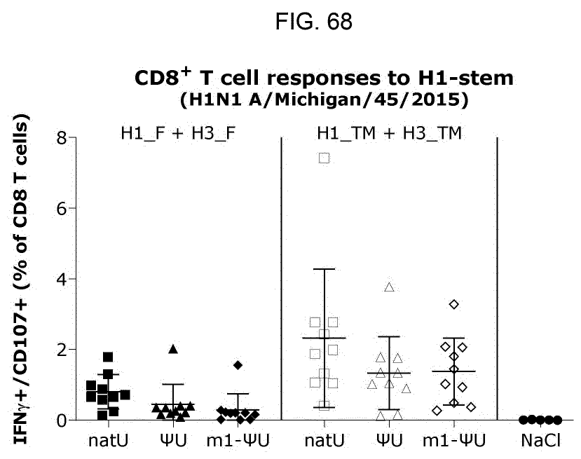

The assay protocol is the same as described in Example 16. Graph depicts IFNγ/CD107 double positive CD8+ T cells, lines indicate mean+/−SD. Results are shown in FIG. 68. TM designs induce higher levels of HA-stem specific antibodies. All designs induce multifunctional H1-specific CD8+ T cells expressing IFNγ, TNF and/or CD107.

### REFERENCES

Corbett, et al. Design of Nanoparticulate Group 2 Influenza Virus Hemagglutinin Stem Antigens That Activate Unmutated Ancestor B Cell Receptors of Broadly Neutralizing Antibody Lineages, mBio. 2019 10(1):e02810-18
Corti, et al. A neutralizing antibody selected from plasma cells that binds to group 1 and group 2 influenza A hemagglutinins, Science 2011 333, 850-856
Dayhoff M, O et al. A model of evolutionary change in proteins, Atlas of Protein Sequence and Structure, 1978 vol 5, supp. 3.
Friesen et al. New class of monoclonal antibodies against severe influenza: prophylactic and therapeutic efficacy in ferrets, PLoS ONE, 5:e9106 (2010).
Jiang S, et al., Lipidoid-Coated Iron Oxide Nanoparticles for Efficient DNA and siRNA delivery Nano Lett., 13:1059-1064 (2013)
Pardi N, et al., mRNA vaccines—a new era in vaccinology, Nat Rev Drug Discov. 2018, 17(4):261-279
Steel J, et al. Influenza virus vaccine based on the conserved hemagglutinin stalk domain, mBio. 2010 1(1):e00018-10
Wu Y, et al. A potent broad-spectrum protective human monoclonal antibody crosslinking two haemagglutinin monomers of influenza A virus, Nat. Commun. 2015, 6:7708
Zhang, C et al. Advances in mRNA vaccines for infectious diseases, Front. Immunol. 2019, 10:594.

1-116. (canceled)

117. Carrier-formulated mRNA comprising at least one coding sequence encoding an influenza HA stem polypeptide.

118. The carrier-formulated mRNA according to claim 117, wherein the carrier is a lipid nanoparticle (LNP).

119. The carrier-formulated mRNA according to claim 118, wherein the LNP comprises a PEG-modified lipid at around 0.5 to 15 molar %, a non-cationic lipid at around 5 to 25 molar %, a sterol at around 25 to 55 molar %, and an ionisable cationic lipid at around 20 to 60 molar %, wherein the ionisable cationic lipid has the formula III: 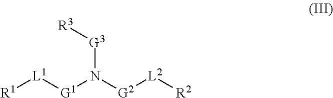
or a pharmaceutically acceptable salt, tautomer or stereoisomer thereof, wherein:
L1 or L2 is each independently —O(C═O)— or —(C═O)O—;
G1 and G2 are each independently unsubstituted C1-C12 alkylene or C1-C12 alkenylene;
G3 is C1-C24 alkylene, C1-C24 alkenylene, C3-C8 cycloalkylene, or C3-C8 cycloalkenylene;
R1 and R2 are each independently C6-C24 alkyl or C6-C24 alkenyl;
R3 is H, OR5, CN, —C(═O)OR4, —OC(═O)R4 or —NR5C(═O)R4;
R4 is C1-C12 alkyl; and
R5 is H or C1-C6 alkyl.

120. The carrier-formulated mRNA according to claim 117, wherein the mRNA comprises at least one additional coding sequence which encodes a protein nanoparticle wherein the protein nanoparticle is ferritin or bacterial ferritin.

121. The carrier-formulated mRNA according to claim 117, wherein the influenza HA stem polypeptide is derived from influenza A.

122. The carrier-formulated mRNA according to claim 121, wherein the influenza HA stem polypeptide is derived from influenza A Group 1, or influenza A subtype H1, H2, H5, H6, H8, H9, H11, H12, H13, H16, H17 or H18, or H1.

123. The carrier-formulated mRNA according to claim 121, wherein the influenza HA stem polypeptide is derived from influenza A Group 2, or influenza A subtype H3, H4, H7, H10, H14 and H15, or H3, H7 or H10.

124. The carrier-formulated mRNA according to claim 117, comprising two or more coding sequences each encoding an influenza HA stem polypeptide, wherein said coding sequences are encoded on separate mRNA molecules.

125. The carrier-formulated mRNA according to claim 124, wherein at least one of said two or more coding sequence encodes an influenza HA stem polypeptide derived from influenza A Group 1, or influenza A subtype H1, H2, H5, H6, H8, H9, H11, H12, H13, H16, H17 and/or H18, or H1; and
at least one of said two or more coding sequence encodes an influenza HA stem polypeptide derived from influenza A Group 2, or influenza A subtype H3, H4, H7, H10, H14 and/or H15, or H3, H7 and/or H10, or H3.

126. The carrier-formulated mRNA according to claim 125, comprising three or more coding sequences each encoding an influenza HA stem polypeptide, at least one of said three or more coding sequence that encodes an influenza HA stem polypeptide derived influenza A subtype H7, wherein the carrier-formulated mRNA does not comprise a coding sequence that encodes an influenza HA stem polypeptide derived influenza A subtype H10.

127. The carrier-formulated mRNA according to claim 125, comprising at least three coding sequences each encoding an influenza HA stem polypeptide, but not comprising a coding sequence that encodes an influenza HA stem polypeptide derived influenza A subtype H10, wherein the carrier-formulated mRNA does not comprise a coding sequence that encodes an influenza HA stem polypeptide derived influenza A subtype H7.

128. The carrier-formulated mRNA according to claim 117, wherein the mRNA comprises a 5′ untranslated region (UTR), wherein the 5′ UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

129. The carrier-formulated mRNA according to claim 117, wherein the mRNA comprises a 3′ UTR, wherein the 3′ UTR comprises or consists of a nucleic acid sequence derived from a 3′-UTR of a gene selected from PSMB3, ALB7, CASP1, COX6B1, GNAS, NDUFA1 and RPS9, or from a homolog, a fragment or a variant of any one of these genes.

130. The carrier-formulated mRNA according to claim 117, wherein the mRNA comprises at least one chemical modification, wherein the chemical modification is N1-methylpseudouridine and/or pseudouridine, or N1-methylpseudouridine.

131. Immunogenic composition comprising the carrier-formulated mRNA according to claim 117, wherein the composition comprises at least one pharmaceutically acceptable carrier.

132. Vaccine comprising the mRNA of claim 117 and/or the immunogenic composition of claim 15.

133. A kit or kit of parts comprising the RNA of claim 117, and/or the composition of claim 131, and/or the vaccine of claim 132.

134. A method of treating or preventing a disorder, wherein the method comprises applying or administering to a subject in need thereof the carrier-formulated mRNA of claim 117, the composition of claim 131, the vaccine of claim 132 or the kit or kit of parts of claim 133.

135. A method of eliciting an immune response against an influenza virus, wherein the method comprises applying or administering to a subject in need thereof the carrier-formulated mRNA of claim 117, the composition of claim 131, the vaccine of claim 132 or the kit or kit of parts of claim 133.
